(19) United States
(12) Patent Application Publication
(10) Pub. No.: US 2024/0216530 A1
(43) Pub. Date: Jul. 4, 2024

(54) Compositions and Methods for Treating Skeletal Muscle Disease

(71) Applicants: Yale University, New Haven, CT (US); Gennao Bio, Inc., Pennington, NJ (US)

(72) Inventors: Elias Quijano, New Haven, CT (US); Peter Glazer, New Haven, CT (US); Bruce C. Turner, Bryn Mawr, PA (US); Stephen Squinto, New York, NY (US)

(21) Appl. No.: 18/548,823

(22) PCT Filed: Mar. 3, 2022
(86) PCT No.: PCT/US2022/018780

§ 371 Date: Sep. 1, 2023

Related U.S. Application Data
(60) Provisional application No. 63/297,504, filed on Jan. 7, 2022.
(60) Provisional application No. 63/156,060, filed on Mar. 3, 2021.

(51) Int. Cl. A61K 48/00; A61K 38/17; A61K 47/68; A61P 21/00; C07K 16/44

(52) U.S. Cl. CPC A61K 48/0025; A61K 38/1719; A61K 47/6807; A61P 21/00; C07K 16/44

(57) ABSTRACT

Compositions and methods are provided for treating myopathies by administering a complex formed between a therapeutic mRNA polynucleotide and a 3E10 antibody or variant thereof, or antigen-binding fragment thereof. In some instances, the complexes are stabilized through a molar ratio of 3E10 antibody or variant thereof, or antigen-binding fragment thereof to therapeutic polynucleotide of at least about 2:1.

### CROSS REFERENCE TO RELATED APPLICATIONS

This application claims priority to U.S. Provisional Patent Application No. 63/156,070, filed Mar. 3, 2021 and U.S. Provisional Patent Application No. 63/297,504, filed Jan. 7, 2022, the contents of which are hereby incorporated by reference in their entireties for all purposes.

### STATEMENT REGARDING FEDERALLY SPONSORED RESEARCH

This invention was made with government support under R35CA197574 awarded by National Institutes of Health. The Government has certain rights in the invention.

### TECHNICAL FIELD

The present disclosure relates generally to compositions and methods for treating skeletal muscle diseases by targeted delivery of mRNA to skeletal muscle tissue.

### BACKGROUND

Myopathies are clinical disorders of the skeletal muscles. These disorders are typically characterized by abnormalities of muscle cell structure and/or metabolism, resulting in various patterns of muscle weakness and dysfunction. There are many types of genetic myopathies, caused by mutations in one or more of a large set of genes. Subjects with genetic myopathies commonly suffer from muscle weakness, motor delay, respiratory impairment, and bulbar muscle dysfunction. Because the etiology of many different forms of genetic myopathies has been well characterized, protein replacement therapies, including nucleic acid-based therapies that deliver a gene or transcript encoding a functional version of the protein to affected tissues offer an attractive option for treating these disorders. In fact, clinical trials for such strategies have been initiated for several genetic myopathies.

Therapeutic mRNA delivery is an attractive option for treating myopathies because it potentially avoids many of the limitations and risks associated with viral vector and synthetic liposome-based gene therapy, including complexity of production, limited packaging capacity, and unfavorable immunological features, which restrict gene therapy applications and hold back the potential for preventive gene therapy (Seow and Wood, Mol Ther. 17(5): 767-777 (2009).

However, mRNA therapy is limited by the need for improved delivery systems. For instance, mRNA does not readily cross the cell membrane. Conventional approaches to overcoming this obstacle include packaging mRNA in liposomal-based delivery vehicles, which present similar immunological challenges as DNA-based therapies. Further, mRNA is readily degraded by extracellular ribonucleases present in skin, tissues, and blood. Kowalski P S et al., Mol Ther., 27(4):710-28 (2019), the content of which is incorporated by reference herein.

### SUMMARY

Given the background above, improved methods for treating skeletal muscle diseases are needed. mRNA therapies present a promising path for treatment of these diseases because the underlying genetics of skeletal muscle disease etiology are well characterized. See, for example, Muscle Cell and Tissue—Current Status of Research Field, Edited by Kunihiro Sakuma, Chapter 6 “Genetic Myopathies” (2018), the content of which is incorporated herein by reference. Advantageously, the present disclosure provides compositions and methods for mRNA therapy of skeletal muscle disease that are not reliant upon liposomal or viral vector based nucleic acid delivery. In some aspects, these compositions and methods are based on, at least in part, on the discovery that 3E10 antibodies or variants thereof, or antigen-binding fragments thereof can be used to efficiently deliver therapeutic mRNA molecules to skeletal muscle tissue in vivo.

In some embodiments, the advantageous properties of the compositions and methods described herein are based, at least in part, on the discovery that 3E10 antibodies or variants thereof, or antigen-binding fragments thereof, as described below, localize to skeletal muscle tissue in vivo (in relation to other tissues or organs) following systemic or intramuscular administration. For instance, as described in Example 3 and illustrated in FIG. 10, following intravenous administration, both parental 3E10 antibody and 3E10 (D31N) variant antibody accumulated at greater concentrations in skeletal muscle than in other non-hepatic tissues, e.g., brain, lung, heart, spleen, and renal tissues. Advantageously, this tropism for skeletal muscle tissue is exploited in the compositions and methods described herein to deliver therapeutic mRNA molecules to skeletal muscle tissue for treatment of various myopathies.

In some embodiments, the advantageous properties of the compositions and methods described herein are based, at least in part, on the discovery that use of higher molar ratios of 3E10 antibody or variant thereof, or antigen-binding fragment thereof to mRNA molecule result in greater protection of the mRNA molecule from RNA degradation. For instance, as described in Example 6 and illustrated in FIGS. 13A and 13B, while parental 3E10 and 3E10 (D31N) variant antibodies protected mRNA from RNAse A-mediated RNA degradation at molar ratios of 2:1 and 20:1, the protection afforded by the 20:1 molar ratio exceeded the protection afforded at 2:1. Advantageously, the increased mRNA protection afforded mRNA at higher 3E10 antibody or variant thereof, or antigen-binding fragment thereof concentrations is exploited in the compositions and methods described herein to improve the pharmacokinetic properties of therapeutic compositions delivering mRNA in vivo.

In some embodiments, the advantageous properties of the compositions and methods described herein are based, at least in part, on the discovery that sustained protein expression in skeletal muscle tissue from a therapeutic mRNA is realized by administration of a complex of the 3E10 antibody or variant thereof, or antigen-binding fragment thereof and the therapeutic mRNA. For instance, as described in Example 5 and illustrated in FIGS. 12A-12B, intramuscular administration of a 3E10 (D31N) variant antibody-mRNA complex resulted in sustained expression of a luciferase encoded by the mRNA for at least five days. Advantageously, the sustained expression in skeletal muscle tissue resulting from administration of these complexes is exploited in the compositions and methods described herein to treat myopathies with long-acting compositions.

Accordingly, one aspect of the present disclosure provides methods for treating a genetic skeletal muscle disease in a subject in need thereof, by parenterally administering a therapeutically effective amount of a composition comprising a complex formed between a therapeutic mRNA polynucleotide, and a 3E10 antibody or variant thereof, or antigen-binding fragment thereof.

In another aspect, the present disclosure provides pharmaceutical compositions of a complex formed between a therapeutic mRNA polynucleotide encoding a skeletal muscle polypeptide, and a 3E10 antibody or variant thereof, or antigen-binding fragment thereof, where the pharmaceutical composition has a molar ratio of 3E10 antibody or variant thereof, or antigen-binding fragment thereof to therapeutic polynucleotide of at least 2:1.

In some embodiments of the methods and compositions described herein, the 3E10 antibody or variant thereof, or antigen-binding fragment thereof includes (a) a light chain variable region (VL) complementarity determining region (CDR) 1 comprising the amino acid sequence of 3E10-VL-CDR1 (SEQ ID NO:9), (b) a VL CDR2 comprising the amino acid sequence of 3E10-VL-CDR2 (SEQ ID NO:10), (c) a VL CDR3 comprising the amino acid sequence of 3E10-VL-CDR3 (SEQ ID NO:11), (d) a heavy chain variable region (VH) CDR1 comprising the amino acid sequence of 3E10-VH-CDR1a (SEQ ID NO:16), (e) a VH CDR2 comprising the amino acid sequence of 3E10-VH-CDR2 (SEQ ID NO:4), and (f) a VH CDR3 comprising the amino acid sequence of 3E10-VH-CDR3 (SEQ ID NO:5).

In some embodiments of the methods and compositions described herein, the 3E10 antibody or variant thereof, or antigen-binding fragment thereof includes (a) a light chain variable region (VL) complementarity determining region (CDR) 1 comprising an amino acid sequence having no more than two amino acid substitutions relative to 3E10-VL-CDR1 (SEQ ID NO:9), (b) a VL CDR2 comprising an amino acid sequence having no more than two amino acid substitutions relative to 3E10-VL-CDR2 (SEQ ID NO:10), (c) a VL CDR3 comprising an amino acid sequence having no more than two amino acid substitutions relative to 3E10-VL-CDR3 (SEQ ID NO:11), (d) a heavy chain variable region (VH) CDR1 comprising an amino acid sequence having no more than two amino acid substitutions relative to 3E10-VH-CDR1a (SEQ ID NO:16), (e) a VH CDR2 comprising an amino acid sequence having no more than two amino acid substitutions relative to 3E10-VH-CDR2 (SEQ ID NO:4), and (f) a VH CDR3 comprising an amino acid sequence having no more than two amino acid substitutions relative to 3E10-VH-CDR3 (SEQ ID NO:5).

In some embodiments of the methods and compositions described herein, the 3E10 antibody or variant thereof, or antigen-binding fragment thereof includes (a) a light chain variable region (VL) complementarity determining region (CDR) 1 comprising the amino acid sequence of 3E10-VL-CDR1m (SEQ ID NO:61), (b) a VL CDR2 comprising the amino acid sequence of 3E10-VL-CDR2m (SEQ ID NO:62), (c) a VL CDR3 comprising the amino acid sequence of 3E10-VL-CDR3m (SEQ ID NO:63), (d) a heavy chain variable region (VH) CDR1 comprising the amino acid sequence of 3E10-VH-CDR1m (SEQ ID NO:58), (e) a VH CDR2 comprising the amino acid sequence of 3E10-VH-CDR2m (SEQ ID NO:59), and (f) a VH CDR3 comprising the amino acid sequence of 3E10-VH-CDR3m (SEQ ID NO:60).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates amino acid sequences for the parent 3E10 monoclonal antibody.

FIGS. 2A, 2B, and 2C illustrate amino acid sequences for the D31N variant (FIG. 2A), other CDR variants (FIG. 2B), and additionally contemplated CDR variants (FIG. 2C) of the 3E10 monoclonal antibody, in accordance with some embodiments of the present disclosure.

FIG. 3 illustrates example charge-conserved CDR variants of the 3E10 monoclonal antibody, in accordance with various embodiments of the present disclosure.

FIG. 4 illustrates example CDR variants containing a combination of amino acid substitutions, charged-conserved amino acid substitutions, and rationally-designed amino acid substitutions of the 3E10 monoclonal antibody, in accordance with various embodiments of the present disclosure.

FIG. 5 illustrates a sequence alignment of examples of humanized 3E10 heavy chain variable regions, with CDRs underlined as indicated.

FIG. 6 illustrates a sequence alignment of examples of humanized 3E10 light chain variable regions, with CDRs and putative nuclear localization signals (NLS) underlined as indicated.

FIGS. 7A, 7B, 7C, 7D, and 7E collectively illustrate a sequence alignment of example of humanized di-scFv constructs of the 3E10 monoclonal antibody.

FIG. 8 illustrates a line graph showing 3E10-mediated delivery of mRNA (bioluminescene (Photons/second)) to mouse muscles (IM) over time (days post-IM injection), in accordance with some embodiments of the present disclosure.

FIGS. 9A, 9B, and 9C collectively show fluorescently-labeled 3E10 (D31N) antibody localization in mouse skeletal muscle following intravenous administration. FIGS. 9A and 9B are images of fluorescence in mouse skeletal muscle following intravenous injections of a control composition (FIG. 9A) or fluorescently-labeled 3E10 (D31N) antibody (FIG. 9B), acquired by IVIS (Perkin Elmer) 24 hours after administration. FIG. 9C is a bar graph quantifying the fluorescence in the IVIS images.

FIG. 10 is a bar graph quantifying the fluorescence in IVIS images of dose-dependent biodistribution of 3E10-D31N to tissues 24 hours following 100 μg or 200 μg intravenous injection of 3E10-D31N labeled with VivoTag680 into mice (Perkin Elmer).

FIGS. 11A and 11B illustrate electrostatic surface potential renderings of a molecular model of a 3E10-scFv construct, revealing a putative Nucleic Acid Binding pocket (NAB1). FIG. 11A additionally shows predicted structural and electrostatic potential changes induced by amino acid substitutions at residue HC CDR1 residue 31. FIG. 11B is an illustration of molecular modeling of 3E10-scFv (Pymol) with NAB1 amino acid residues highlighted by punctate dots.

FIG. 11C illustrates mapping of the putative nucleic acid binding pocket, as identified by the molecular modeling shown in FIGS. 11A and 11B, onto the amino acid sequence of the 3E10-scFv construct.

FIGS. 12A and 12B show expression of mRNA in skeletal muscle following intramuscular administration of a 3E10 (D31N)-mRNA construct. FIG. 12A show fluorescent images of a mouse over a five-day time course following intramuscular administration of mRNA encoding a luciferase complexed with 3E10 (D31N). FIG. 12B illustrates a bar graph quantifying average radiance over all pixels, showing fluorescence in single mice in images of control mice (untreated) and mice administered the 3E10 (D31N)-mRNA construct intramuscularly.

FIGS. 13A and 13B show gel electrophoresis analysis mRNA protection assays performed with 3E10 (D31N)-mRNA constructs prepared at 20:1 (FIG. 13A) and 2:1 (FIG. 13B) molar ratios.

FIG. 14 shows a histogram of cytosolic, membrane, nuclear protein, and gDNA fractions after administration of ⁸⁹Zr labeled isotype control, 3E10-WT, and 3E10-D31N antibodies, as described in Example 7.

FIG. 15 shows gel electrophoresis analysis of mRNA protection assays performed with complexes formed between 3E10 and a 14 kb mRNA encoding the human dystrophin protein, prepared at 1:1, 2:1, 5:1, 10:1 and 100:1 (3E10:mRNA) molar ratios, as described in Example 8.

### DETAILED DESCRIPTION

The present disclosure provides compositions and methods for delivering therapeutic mRNA molecules, in vivo, that are not reliant upon the conventional viral-based or liposomal-based delivery methodologies associated with difficult and costly production, limited packaging capacity, and adverse immunological events. In some aspects, described in greater detail below, these compositions and methods are based on, at least in part, on the discovery that 3E10 antibodies or variants thereof, or antigen-binding fragments thereof can be used to deliver therapeutic mRNA molecules efficiently to skeletal muscle tissue in vivo.

Specifically, it was discovered that 3E10 antibodies or variants thereof, or antigen-binding fragments thereof help transport mRNA across the plasma membrane, into the cell cytoplasm. Thus, compositions and methods for using 3E10 antibodies or variants thereof, or antigen-binding fragments thereof to enhance delivery of mRNA, particularly to skeletal muscle tissue, are provided.

### Definitions

The terminology used in the present disclosure is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. Unless the context requires otherwise, it will be further understood that the terms “includes,” “comprising,” or any variation thereof, when used in this specification, specify the presence of stated features, integers, steps, operations, elements, and/or components, but do not preclude the presence or addition of one or more other features, integers, steps, operations, elements, components, and/or groups thereof. Furthermore, to the extent that the terms “including,” “includes,” “having,” “has,” “with,” or variants thereof are used in either the detailed description and/or the claims, such terms are intended to be inclusive in a manner similar to the term “comprising.”

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%.

By “antigen binding domain” or “ABD” herein is meant a set of six Complementary Determining Regions (CDRs) that, when present as part of a polypeptide sequence or sequences, specifically binds a target antigen as discussed herein. Thus, a “nucleic acid binding domain” binds a nucleic acid antigen as outlined herein. As is known in the art, these CDRs are generally present as a first set of variable heavy CDRs (vhCDRs or VHCDRs) and a second set of variable light CDRs (vlCDRs or VLCDRs), each comprising three CDRs: vhCDR1, vhCDR2, vhCDR3 for the heavy chain and vlCDR1, vlCDR2 and vlCDR3 for the light. The CDRs are present in the variable heavy and variable light domains, respectively, and together form an Fv region. (See Table 1 and related discussion above for CDR numbering schemes). Thus, in some cases, the six CDRs of the antigen binding domain are contributed by a variable heavy and a variable light domain. In a “Fab” format, the set of 6 CDRs are contributed by two different polypeptide sequences, the variable heavy domain (vh or VH; containing the vhCDR1, vhCDR2 and vhCDR3) and the variable light domain (vl or VL; containing the vlCDR1, vlCDR2 and vlCDR3), with the C-terminus of the vh domain being attached to the N-terminus of the CH1 domain of the heavy chain and the C-terminus of the vl domain being attached to the N-terminus of the constant light domain (and thus forming the light chain). In a scFv format, the vh and vl domains are covalently attached, generally through the use of a linker (a “scFv linker”) as outlined herein, into a single polypeptide sequence, which can be either (starting from the N-terminus) vh-linker-vl or vl-linker-vh, with the former being generally preferred (including optional domain linkers on each side, depending on the format used. In general, the C-terminus of the scFv domain is attached to the N-terminus of the hinge in the second monomer.

As will be appreciated by those in the art, the exact numbering and placement of the CDRs can be different among different numbering systems. However, it should be understood that the disclosure of a variable heavy and/or variable light sequence includes the disclosure of the associated (inherent) CDRs. Accordingly, the disclosure of each variable heavy region is a disclosure of the vhCDRs (e.g. vhCDR1, vhCDR2 and vhCDR3) and the disclosure of each variable light region is a disclosure of the vlCDRs (e.g. vlCDR1, vlCDR2 and vlCDR3). A useful comparison of CDR numbering is as below, see Lafranc et al., Dev. Comp. Immunol. 27(1):55-77 (2003):

TABLE 1

| | Kabat + Chothia | IMGT | Kabat | AbM | Chothia | Contact | Xencor |
|---|---|---|---|---|---|---|---|
| vhCDR1 | 26-35 | 27-38 | 31-35 | 26-35 | 26-32 | 30-35 | 27-35 |
| vhCDR2 | 50-65 | 56-65 | 50-65 | 50-58 | 52-56 | 47-58 | 54-61 |
| vhCDR3 | 95-102 | 105-117 | 95-102 | 95-102 | 95-102 | 93-101 | 103-116 |
| vlCDR1 | 24-34 | 27-38 | 24-34 | 24-34 | 24-34 | 30-36 | 27-38 |
| vlCDR2 | 50-56 | 56-65 | 50-56 | 50-56 | 50-56 | 46-55 | 56-62 |
| vlCDR3 | 89-97 | 105-117 | 89-97 | 89-97 | 89-97 | 89-96 | 97-105 |

Throughout the present specification, the Kabat numbering system is generally used when referring to a residue in the variable domain (approximately, residues 1-107 of the light chain variable region and residues 1-113 of the heavy chain variable region) and the EU numbering system for Fc regions (e.g, Kabat et al., supra (1991)). The EU index or EU index as in Kabat or EU numbering scheme refers to the numbering of the EU antibody. Kabat et al. collected numerous primary sequences of the variable regions of heavy chains and light chains. Based on the degree of conservation of the sequences, they classified individual primary sequences into the CDR and the framework and made a list thereof. See, SEQUENCES OF IMMUNOLOGICAL INTEREST, 5th edition, NIH publication. No. 91-3242, E. A. Kabat et al.; Edelman et al., 1969, Proc Natl Acad Sci USA 63:78-85, the contents of which are incorporated herein by reference. The modification can be an addition, deletion, or substitution.

By “target antigen” as used herein is meant the molecule that is bound specifically by the antigen binding domain comprising the variable regions of a given antibody. As discussed below, in the present case the target antigens are nucleic acids.

As described below, in some embodiments a parent polypeptide, for example an Fc parent polypeptide, is a human wild type sequence, such as the heavy constant domain or Fc region from IgG1, IgG2, IgG3 or IgG4, although human sequences with variants can also serve as “parent polypeptides”, for example the IgG½ hybrid of US Publication 2006/0134105 can be included. The protein variant sequence herein will preferably possess at least about 75% identity with a parent protein sequence, or at least about 80% identity with a parent protein sequence, and most preferably at least about 90% identity, more preferably at least about 95%, or at least about 98%, or at least about 99% sequence identity. In some embodiments, the protein variant sequence herein has at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% sequence identity with a parent protein sequence. Accordingly, by “antibody variant” or “variant antibody” as used herein is meant an antibody that differs from a parent antibody by virtue of at least one amino acid modification, “IgG variant” or “variant IgG” as used herein is meant an antibody that differs from a parent IgG (again, in many cases, from a human IgG sequence) by virtue of at least one amino acid modification, and “immunoglobulin variant” or “variant immunoglobulin” as used herein is meant an immunoglobulin sequence that differs from that of a parent immunoglobulin sequence by virtue of at least one amino acid modification. “Fc variant” or “variant Fc” as used herein is meant a protein comprising an amino acid modification in an Fc domain as compared to an Fc domain of human IgG1, IgG2, IgG3, or IgG4.

By “isotype” as used herein is meant any of the subclasses of immunoglobulins defined by the chemical and antigenic characteristics of their constant regions. It should be understood that therapeutic antibodies can also comprise hybrids of isotypes and/or subclasses.

By “Fab” or “Fab region” as used herein is meant a polypeptide that comprises the VH, CH1, VL, and CL immunoglobulin domains, generally on two different polypeptide chains (e.g. VH-CH1 on one chain and VL-CL on the other). Fab may refer to this region in isolation, or this region in the context of an antibody of the disclosure. In the context of a Fab, the Fab comprises an Fv region in addition to the CH1 and CL domains.

By “Fv” or “Fv fragment” or “Fv region” as used herein is meant a polypeptide that comprises the VL and VH domains of an ABD. Fv regions can be formatted as both Fabs (as discussed above, generally two different polypeptides that also include the constant regions as outlined above) and scFvs, where the vl and vh domains are combined (generally with a linker as discussed herein) to form an scFv.

By “single chain Fv” or “scFv” herein is meant a variable heavy domain covalently attached to a variable light domain, generally using a scFv linker as discussed herein, to form a scFv or scFv domain. A scFv domain can be in either orientation from N- to C-terminus (vh-linker-vl or vl-linker-vh). In the sequences depicted in the sequence listing and in the figures, the order of the vh and vl domain is indicated in the name, e.g. H.X_L. Y means N- to C-terminal is vh-linker-vl, and L. Y_H.X is vl-linker-vh.

By “Fc” or “Fc region” or “Fc domain” as used herein is meant the polypeptide comprising the CH2-CH3 domains of an IgG molecule, and in some cases, inclusive of the hinge. In EU numbering for human IgG1, the CH2-CH3 domain comprises amino acids 231 to 447, and the hinge is 216 to 230. Thus the definition of “Fc domain” includes both amino acids 231-447 (CH2-CH3) or 216-447 (hinge-CH2-CH3), or fragments thereof. An “Fc fragment” in this context may contain fewer amino acids from either or both of the N- and C-termini but still retains the ability to form a dimer with another Fc domain or Fc fragment as can be detected using standard methods, generally based on size (e.g. non-denaturing chromatography, size exclusion chromatography, etc.) Human IgG Fc domains are of particular use in the present disclosure, and can be the Fc domain from human IgG1, IgG2 or IgG4.

A “variant Fc domain” contains amino acid modifications as compared to a parental Fc domain. Thus, a “variant human IgG1 Fc domain” is one that contains amino acid modifications (generally amino acid substitutions, although in the case of ablation variants, amino acid deletions are included) as compared to the human IgG1 Fc domain. In general, variant Fc domains have at least about 80, about 85, about 90, about 95, about 97, about 98 or about 99 percent identity to the corresponding parental human IgG Fc domain (using the identity algorithms discussed below, with one embodiment utilizing the BLAST algorithm as is known in the art, using default parameters). Alternatively, the variant Fc domains can have from 1 to about 20 (e.g., 1, 2, 3, 4, 5, 6, 7, 8, 9, 10, 11, 12, 13, 14, 15, 16, 17, 18, 19 or 20) amino acid modifications as compared to the parental Fc domain. Additionally, as discussed herein, the variant Fc domains herein still retain the ability to form a dimer with another Fc domain as measured using known techniques as described herein, such as non-denaturing gel electrophoresis.

By “heavy chain constant region” herein is meant the CH1-hinge-CH2-CH3 portion of an antibody (or fragments thereof), excluding the variable heavy domain; in EU numbering of human IgG1 this is amino acids 118-447 By “heavy chain constant region fragment” herein is meant a heavy chain constant region that contains fewer amino acids from either or both of the N- and C-termini but still retains the ability to form a dimer with another heavy chain constant region.

By “variable region” or “variable domain” as used herein is meant the region of an immunoglobulin that comprises one or more Ig domains substantially encoded by any of the VK, VA, and/or VH genes that make up the kappa, lambda, and heavy chain immunoglobulin genetic loci respectively, and contains the CDRs that confer antigen specificity. Thus, a “variable heavy domain” pairs with a “variable light domain” to form an antigen binding domain (“ABD”). In addition, each variable domain comprises three hypervariable regions (“complementary determining regions,” “CDRs”) (vhCDR1, vhCDR2 and vhCDR3 for the variable heavy domain and vlCDR1, vlCDR2 and vlCDR3 for the variable light domain) and four framework (FR) regions, arranged from amino-terminus to carboxy-terminus in the following order: FR1-CDR1-FR2-CDR2-FR3-CDR3-FR4.

By “IgG subclass modification” or “isotype modification” as used herein is meant an amino acid modification that converts one amino acid of one IgG isotype to the corresponding amino acid in a different, aligned IgG isotype. For example, because IgG1 comprises a tyrosine and IgG2 a phenylalanine at EU position 296, a F296Y substitution in IgG2 is considered an IgG subclass modification.

By “non-naturally occurring modification” as used herein is meant an amino acid modification that is not isotypic. For example, because none of the human IgGs comprise a serine at position 434, the substitution 434S in IgG1, IgG2, IgG3, or IgG4 (or hybrids thereof) is considered a non-naturally occurring modification.

The antibodies of the present disclosure are generally isolated or recombinant. “Isolated,” when used to describe the various polypeptides disclosed herein, means a polypeptide that has been identified and separated and/or recovered from a cell or cell culture from which it was expressed. Ordinarily, an isolated polypeptide will be prepared by at least one purification step. An “isolated antibody,” refers to an antibody which is substantially free of other antibodies having different antigenic specificities. “Recombinant” means the antibodies are generated using recombinant nucleic acid techniques in exogenous host cells, and they can be isolated as well.

As used herein, the term “cell-penetrating antibody” refers to an immunoglobulin protein, fragment, variant thereof, or fusion protein based thereon that is transported into the cytoplasm and/or nucleus of living mammalian cells. The “cell-penetrating anti-DNA antibody” specifically binds DNA (e.g., single-stranded and/or double-stranded DNA). In some embodiments, the antibody is transported into the cytoplasm of the cells without the aid of a carrier or conjugate. In other embodiments, the antibody is conjugated to a cell-penetrating moiety, such as a cell penetrating peptide. In some embodiments, the cell-penetrating antibody is transported in the nucleus with or without a carrier or conjugate.

By “skeletal muscle polypeptide” herein is meant a polypeptide having a substantially similar structure and function as a protein, or polypeptide chain thereof, that is genetically-linked to a skeletal muscle disease, e.g., a protein, or polypeptide chain thereof, for which mutations exist that result in a skeletal muscle disease. The term “skeletal muscle polypeptide” encompasses wild type versions of skeletal muscle proteins, and polypeptide chains thereof, natural variant versions of skeletal muscle proteins, and polypeptide chains thereof, as well as engineered versions of skeletal muscle proteins, and polypeptide chains thereof. Skeletal muscle polypeptides are also intended to encompass proteins, and polypeptide chains thereof, having a function that partially or completely rescues a function lost by a mutation in a protein, or polypeptide chain thereof, genetically-linked to a skeletal muscle disease, including but not limited to various homologues of a skeletal muscle protein, or polypeptide chain thereof.

By “modification” herein is meant an amino acid substitution, insertion, and/or deletion in a polypeptide sequence.

By “variant protein” or “protein variant”, or “variant” as used herein is meant a protein that differs from that of a parent protein by virtue of at least one amino acid modification. The protein variant has at least one amino acid modification compared to the parent protein, yet not so many that the variant protein will not align with the parental protein using an alignment program such as that described below. In general, variant proteins (such as variant Fc domains, etc., outlined herein, are generally at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the parent protein, using the alignment programs described below, such as BLAST.

Sequence identity between two similar sequences (e.g., antibody variable domains) can be measured by algorithms such as that of Smith, T. F. & Waterman, M. S. (1981) “Comparison Of Biosequences,” Adv. Appl. Math. 2:482 [local homology algorithm]; Needleman, S. B. & Wunsch, CD. (1970) “A General Method Applicable To The Search For Similarities In The Amino Acid Sequence Of Two Proteins,” J. Mol. Biol., 48:443 [homology alignment algorithm], Pearson, W. R. & Lipman, D. J. (1988) “Improved Tools For Biological Sequence Comparison,” Proc. Natl. Acad. Sci. (U.S.A.) 85:2444 [search for similarity method]; or Altschul, S. F. et al, (1990) “Basic Local Alignment Search Tool,” J. Mol. Biol. 215:403-10, the “BLAST” algorithm, see the webpage located at URL blast.ncbi.nlm.nih.gov/Blast.cgi. When using any of the aforementioned algorithms, the default parameters (for Window length, gap penalty, etc.) are used. Unless specifically stated otherwise, sequence identity is determined using the BLAST algorithm, using default parameters

As used herein, the term “subject” means any individual who is the target of administration. The subject can be a vertebrate, for example, a mammal. Thus, the subject can be a human. The term does not denote a particular age or sex.

As used herein, the term “pharmaceutically effective amount” means that the amount of the composition used is of sufficient quantity to ameliorate one or more causes or symptoms of a disease or disorder. Such amelioration only requires a reduction or alteration, not necessarily elimination. The precise dosage will vary according to a variety of factors such as subject-dependent variables (e.g., age, immune system health, etc.), the disease or disorder being treated, as well as the route of administration and the pharmacokinetics of the agent being administered.

As used herein, the term “carrier” or “excipient” refers to an organic or inorganic ingredient, natural or synthetic inactive ingredient in a formulation, with which one or more active ingredients are combined. The carrier or excipient would naturally be selected to minimize degradation of the active ingredient or to minimize adverse side effects in the subject, as would be well known to one of skill in the art.

As used herein, the term “treat” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

As used herein, the term “genetic skeletal muscle disease” refers to a disorder having a genetic basis that primarily affects the skeletal muscle tissue. Genetic myopathies are caused by mutations in various genes encoding proteins that function in muscle structure and function. Genetic myopathies typically manifest as skeletal muscle weakness and hypotonia. Non-limiting examples of different types of genetic myopathies are provided in Table 2.

### 3E10 Antibodies, Variants, and Fragments Thereof

In some aspects, the present disclosure relates to the use of 3E10 antibodies, and derivatives thereof, for delivering therapeutic mRNA molecules to skeletal muscle tissue in a subject, e.g., to treat a genetic skeletal muscle disease. As is discussed below, the term antibody is used generally. Antibodies that find use in the present disclosure take on a number of formats as described herein, including traditional antibodies as well as antibody derivatives, fragments, and mimetics, described herein in various embodiments.

Traditional antibody structural units typically comprise a tetramer. Each tetramer is typically composed of two identical pairs of polypeptide chains, each pair having one “light” (typically having a molecular weight of about 25 kDa) and one “heavy” chain (typically having a molecular weight of about 50-70 kDa). Human light chains are classified as kappa and lambda light chains. The present disclosure is directed to antibodies that generally are based on the IgG class, which has several subclasses, including, but not limited to IgG1, IgG2, IgG3, and IgG4. In general, IgG1, IgG2 and IgG4 are used more frequently than IgG3. It should be noted that IgG1 has different allotypes with polymorphisms at 356 (D or E) and 358 (L or M).

The light chain generally comprises two domains, the variable light domain (containing the light chain CDRs and together with the variable heavy domains forming the Fv region), and a constant light chain region (often referred to as CL or C_{κ}). The heavy chain comprises a variable heavy domain and a constant domain, which includes a CH1-optional hinge-Fc domain comprising a CH2-CH3.

The hypervariable region of an antibody generally encompasses amino acid residues from about amino acid residues 24-34 (LCDR1; “L” denotes light chain), 50-56 (LCDR2) and 89-97 (LCDR3) in the light chain variable region and around about 31-35B (HCDR1; “H” denotes heavy chain), 50-65 (HCDR2), and 95-102 (HCDR3) in the heavy chain variable region; Kabat et al., SEQUENCES OF PROTEINS OF IMMUNOLOGICAL INTEREST, 5th Ed. Public Health Service, National Institutes of Health, Bethesda, Md. (1991) and/or those residues forming a hypervariable loop (e.g. residues 26-32 (LCDR1), 50-52 (LCDR2) and 91-96 (LCDR3) in the light chain variable region and 26-32 (HCDR1), 53-55 (HCDR2) and 96-101 (HCDR3) in the heavy chain variable region; Chothia and Lesk (1987) J. Mol. Biol. 196:901-917. Specific CDRs useful for the compositions and methods described herein are described below.

As will be appreciated by those in the art, the exact numbering and placement of the CDRs can be different among different numbering systems. However, it should be understood that the disclosure of a variable heavy and/or variable light sequence includes the disclosure of the associated (inherent) CDRs. Accordingly, the disclosure of each variable heavy region is a disclosure of the vhCDRs (e.g. vhCDR1, vhCDR2 and vhCDR3) and the disclosure of each variable light region is a disclosure of the vlCDRs (e.g. vlCDR1, vlCDR2 and vlCDR3). A useful comparison of CDR numbering is described in Lafranc et al., Dev. Comp. Immunol. 27(1):55-77 (2003).

Throughout the present specification, the Kabat numbering system is generally used when referring to a residue in the variable domain (approximately, residues 1-107 of the light chain variable region and residues 1-113 of the heavy chain variable region) and the EU numbering system for Fc regions (e.g., Kabat et al., supra (1991)).

The present disclosure provides a large number of different CDR sets. In this case, a “full CDR set” comprises the three variable light and three variable heavy CDRs, e.g. a vlCDR1, vlCDR2, vlCDR3, vhCDR1, vhCDR2 and vhCDR3. These can be part of a larger variable light or variable heavy domain, respectfully. In addition, as more fully outlined herein, the variable heavy and variable light domains can be on separate polypeptide chains, when a heavy and light chain is used (for example when Fabs are used), or on a single polypeptide chain in the case of scFv sequences.

The CDRs contribute to the formation of the antigen-binding, or more specifically, epitope binding site of antibodies. “Epitope” refers to a determinant that interacts with a specific antigen binding site in the variable region of an antibody molecule known as a paratope. Epitopes are groupings of molecules such as nucleic acids, amino acids, or sugar side chains and usually have specific structural characteristics, as well as specific charge characteristics. A single antigen may have more than one epitope. The antibodies described herein bind to nucleic acid epitopes in a partially sequence-independent manner. That is, while the antibodies described herein bind to some polynucleotide structures and sequences with greater affinity than other nucleic acid structures and sequences, they have some general affinity for polynucleotides.

The “Fc domain” of the heavy chain includes the —CH2-CH3 domain, and optionally a hinge domain (—H—CH2-CH3). For IgG, the Fc domain comprises immunoglobulin domains CH2 and CH3 (Cγ2 and Cγ3) and the lower hinge region between CH1 (Cγ1) and CH2 (Cγ2). Although the boundaries of the Fc region may vary, the human IgG heavy chain Fc region is usually defined to include residues C226 or P230 to its carboxyl-terminus, wherein the numbering is according to the EU index as in Kabat. Accordingly, “CH” domains in the context of IgG are as follows: “CH1” refers to positions 118-215 according to the EU index as in Kabat. “Hinge” refers to positions 216-230 according to the EU index as in Kabat. “CH2” refers to positions 231-340 according to the EU index as in Kabat, and “CH3” refers to positions 341-447 according to the EU index as in Kabat. Thus, the “Fc domain” includes the —CH2-CH3 domain, and optionally a hinge domain (hinge-CH2-CH3). In the embodiments herein, when a scFv is attached to an Fc domain, it is generally the C-terminus of the scFv construct that is attached to all or part of the hinge of the Fc domain; for example, it is generally attached to the sequence EPKS which is the beginning of the hinge. In some embodiments, as is more fully described below, amino acid modifications are made to the Fc region, for example to alter binding to one or more FcγR receptors or to the FcRn receptor, and to enable heterodimer formation and purification, as outlined herein.

Another part of the heavy chain is the hinge region. By “hinge” or “hinge region” or “antibody hinge region” or “hinge domain” herein is meant the flexible polypeptide comprising the amino acids between the first and second constant domains of an antibody. Structurally, the IgG CH1 domain ends at EU position 215, and the IgG CH2 domain begins at residue EU position 231. Thus for IgG the antibody hinge is herein defined to include positions 216 (E216 in IgG1) to 230 (p230 in IgG1), wherein the numbering is according to the EU index as in Kabat. In some cases, a “hinge fragment” is used, which contains fewer amino acids at either or both of the N- and C-termini of the hinge domain.

A scFv comprises a variable heavy chain, an scFv linker, and a variable light domain. In most of the constructs and sequences outlined herein, the C-terminus of the variable heavy chain is attached to the N-terminus of the scFv linker, the C-terminus of which is attached to the N-terminus of a variable light chain (N-vh-linker-vl-C) although that can be switched (N-vl-linker-vh-C).

Thus, the present disclosure relates to different antibody domains. As described herein and known in the art, the heterodimeric antibodies described in certain embodiments of the disclosure comprise different domains within the heavy and light chains, which can be overlapping as well. These domains include, but are not limited to, the Fc domain, the CH1 domain, the CH2 domain, the CH3 domain, the hinge domain, the heavy constant domain (CH1-hinge-Fc domain or CH1-hinge-CH2-CH3), the variable heavy domain, the variable light domain, the light constant domain, Fab domains and scFv domains.

In certain embodiments, the antibodies of the disclosure comprise a heavy chain variable region from a particular germline heavy chain immunoglobulin gene and/or a light chain variable region from a particular germline light chain immunoglobulin gene. For example, such antibodies may comprise or consist of a human antibody comprising heavy or light chain variable regions that are “the product of” or “derived from” a particular germline sequence, e.g., that of the 3E10 antibody. A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody (using the methods outlined herein). A human antibody that is “the product of” or “derived from” a particular human germline immunoglobulin sequence may contain amino acid differences as compared to the germline sequence, due to, for example, naturally-occurring somatic mutations or intentional introduction of site-directed mutation. However, a humanized antibody typically is at least 90% identical in amino acids sequence to an amino acid sequence encoded by a human germline immunoglobulin gene and contains amino acid residues that identify the antibody as being derived from human sequences when compared to the germline immunoglobulin amino acid sequences of other species (e.g., murine germline sequences). In certain cases, a humanized antibody may be at least 95, 96, 97, 98 or 99%, or even at least 96%, 97%, 98%, or 99% identical in amino acid sequence to the amino acid sequence encoded by the germline immunoglobulin gene. Typically, a humanized antibody derived from a particular human germline sequence will display no more than 10-20 amino acid differences from the amino acid sequence encoded by the human germline immunoglobulin gene. In certain cases, the humanized antibody may display no more than 5, or even no more than 4, 3, 2, or 1 amino acid difference from the amino acid sequence encoded by the germline immunoglobulin gene.

In one embodiment, the parent antibody has been affinity matured, as is known in the art. Structure-based methods may be employed for humanization and affinity maturation, for example as described in U.S. Ser. No. 11/004,590, which is incorporated herein by reference. Selection based methods may be employed to humanize and/or affinity mature antibody variable regions, including but not limited to methods described in Wu et al., 1999, J. Mol. Biol. 294:151-162; Baca et al., 1997, J. Biol. Chem. 272(16): 10678-10684; Rosok et al., 1996, J. Biol. Chem. 271(37): 22611-22618; Rader et al., 1998, Proc. Natl. Acad. Sci. USA 95: 8910-8915; Krauss et al., 2003, Protein Engineering 16(10):753-759, all of which are incorporated herein by reference. Other humanization methods may involve the grafting of only parts of the CDRs, including but not limited to methods described in U.S. Ser. No. 09/810,510; Tan et al., 2002, J. Immunol. 169:1119-1125; De Pascalis et al., 2002, J. Immunol. 169:3076-3084, all of which are incorporated herein by reference.

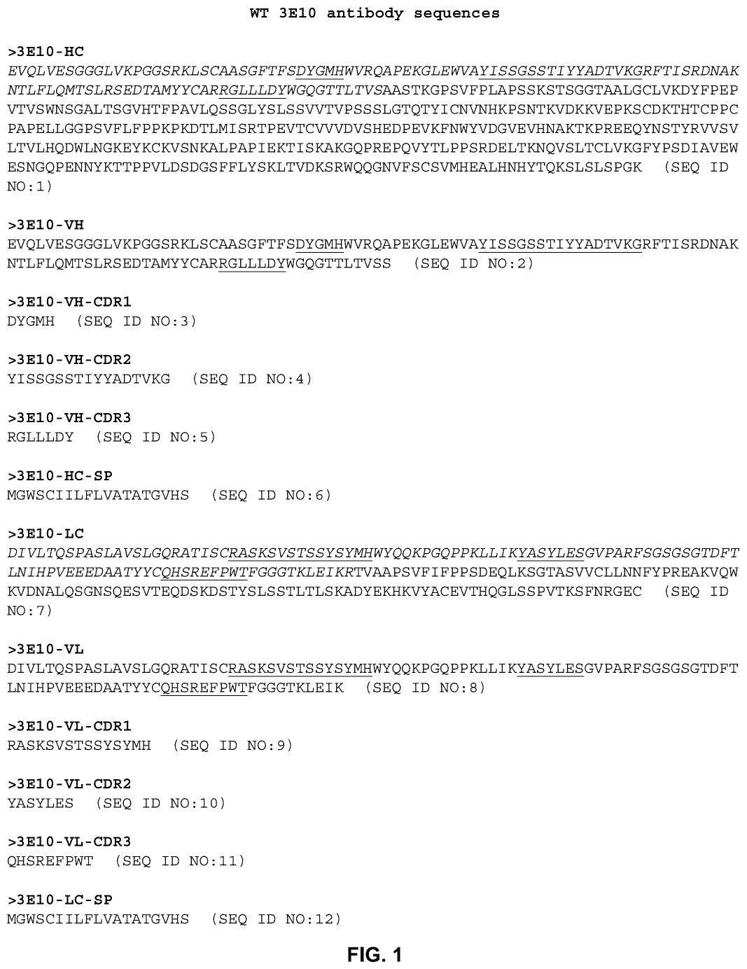

In some aspects, the disclosure relates to the use of antigen binding domains (ABDs) that bind to nucleic acids, and specifically that bind to mRNA molecules, derived from the 3E10 antibody. The amino acid sequence of the heavy and light chains of the parent 3E10 antibody are shown in FIG. 1. Accordingly, in some embodiments, the compositions described herein include a 3E10 antibody or variant thereof, or antigen-binding fragment thereof.

In some embodiments, a 3E10 antibody or variant thereof, or antigen-binding fragment thereof described herein includes CDR sequences corresponding to the parent 3E10 antibody, shown in FIG. 1. Accordingly, in some embodiments, the a 3E10 antibody or variant thereof, or antigen-binding fragment thereof includes a light chain variable region (VL) complementarity determining region (CDR) 1 comprising the amino acid sequence of 3E10-VL-CDR1 (SEQ ID NO:9), a VL CDR2 comprising the amino acid sequence of 3E10-VL-CDR2 (SEQ ID NO:10), a VL CDR3 comprising the amino acid sequence of 3E10-VL-CDR3 (SEQ ID NO:11), a heavy chain variable region (VH) CDR1 comprising the amino acid sequence of 3E10-VH-CDR1 (SEQ ID NO:3), a VH CDR2 comprising the amino acid sequence of 3E10-VH-CDR2 (SEQ ID NO:4), and a VH CDR3 comprising the amino acid sequence of 3E10-VH-CDR3 (SEQ ID NO:5).

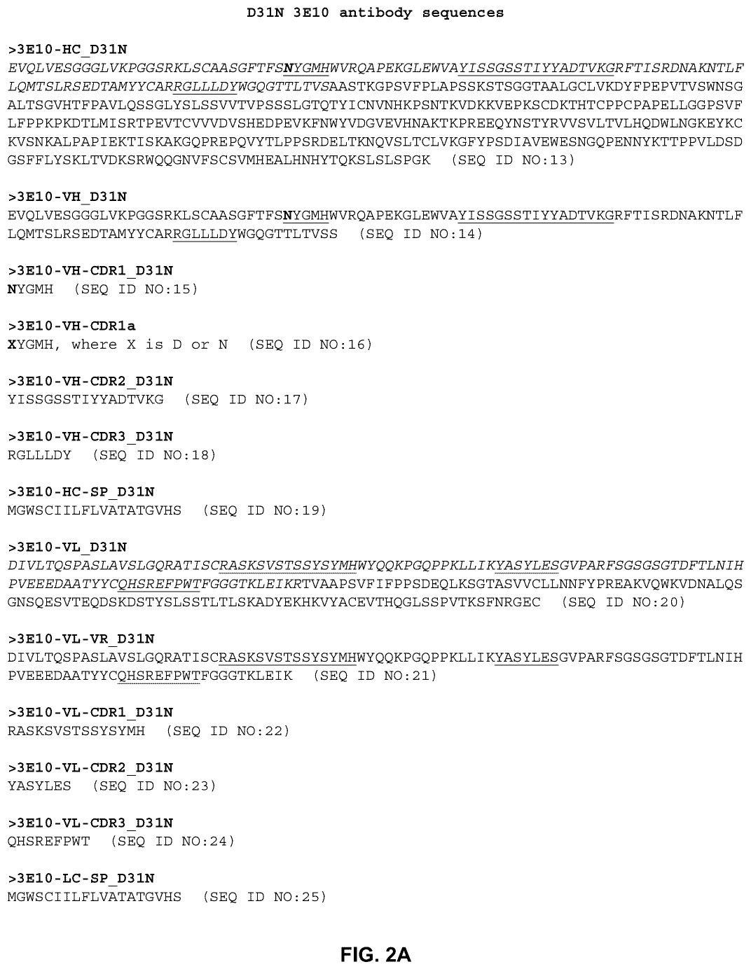

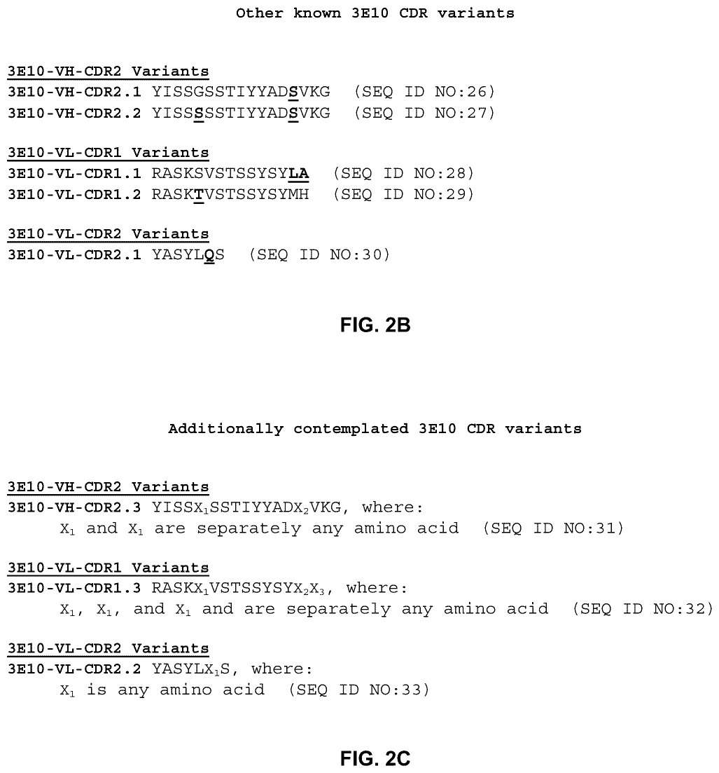

In some embodiments, a 3E10 antibody or variant thereof, or antigen-binding fragment thereof described herein includes CDR sequences from a variant 3E10 antibody that includes a D31N amino acid substitution in the VH CDR1, as shown in FIG. 2. Accordingly, in some embodiments, the a 3E10 antibody or variant thereof, or antigen-binding fragment thereof includes a light chain variable region (VL) complementarity determining region (CDR) 1 comprising the amino acid sequence of 3E10-VL-CDR1_D31N (SEQ ID NO:22), a VL CDR2 comprising the amino acid sequence of 3E10-VL-CDR2_D31N (SEQ ID NO:23), a VL CDR3 comprising the amino acid sequence of 3E10-VL-CDR3_D31N (SEQ ID NO:24), a heavy chain variable region (VH) CDR1 comprising the amino acid sequence of 3E10-VH-CDR1 D31N (SEQ ID NO: 15), a VH CDR2 comprising the amino acid sequence of 3E10-VH-CDR2 D31N (SEQ ID NO: 17), and a VH CDR3 comprising the amino acid sequence of 3E10-VH-CDR3_D31N (SEQ ID NO:18).

In some embodiments, a 3E10 antibody or variant thereof, or antigen-binding fragment thereof described herein refers to CDR sequences corresponding to the parent 3E10 antibody, shown in FIG. 1, optionally including a D31N amino acid substitution in the VH CDR1. Accordingly, in some embodiments, a 3E10 antibody or variant thereof, or antigen-binding fragment thereof includes a light chain variable region (VL) complementarity determining region (CDR) 1 comprising the amino acid sequence of 3E10-VL-CDR1 (SEQ ID NO:9), a VL CDR2 comprising the amino acid sequence of 3E10-VL-CDR2 (SEQ ID NO:10), a VL CDR3 comprising the amino acid sequence of 3E10-VL-CDR3 (SEQ ID NO:11), a heavy chain variable region (VH) CDR1 comprising the amino acid sequence of 3E10-VH-CDR1a (SEQ ID NO:16), a VH CDR2 comprising the amino acid sequence of 3E10-VH-CDR2 (SEQ ID NO:10), and a VH CDR3 comprising the amino acid sequence of 3E10-VH-CDR3 (SEQ ID NO:11).

In some embodiments, a 3E10 antibody or variant thereof, or antigen-binding fragment thereof described herein includes CDR sequences corresponding to the parent 3E10 antibody, shown in FIG. 1, with a known amino acid substitution in one or more CDR. For example, FIG. 2B shows the amino acid sequence of several known VH CDR2, VL CDR1, and VL CDR2 amino acid sequences. Accordingly, in some embodiments, a 3E10 antibody or variant thereof, or antigen-binding fragment thereof described herein includes one or more amino acid substitution, relative to the CDR sequences of the parent 3E10 (shown in FIG. 1) or 3E10-D31N variant (shown in FIG. 2), selected from a G to S substitution at position 5 of VH CDR2, a T to S substitution at position 14 of VH CDR2, an S to T substitution at position 5 of VL CDR1, an M to L substitution at position 14 of VL CDR1, an H to A substitution at position 15 of VL CDR1, and an E to Q substitution at position 6 of VL CDR2.

Accordingly, in some embodiments, a 3E10 antibody or variant thereof, or antigen-binding fragment thereof includes VH CDR2 comprising the amino acid sequence of 3E10-VH-CDR2.1 (SEQ ID NO:26) or 3E10-VH-CDR2.2 (SEQ ID NO:27). In some embodiments, the 3E10 antibody or variant thereof, or antigen-binding fragment thereof further includes VL CDRs 1-3, and VH CRDs 1 and 3 according to the parent 3E10 antibody (as shown in FIG. 1). In some embodiments, the 3E10 antibody or variant thereof, or antigen-binding fragment thereof further includes VL CDRs 1-3, and VH CRDs 1 and 3 according to the 3E10-D31N variant (as shown in FIG. 2A). In some embodiments, the 3E10 antibody or variant thereof, or antigen-binding fragment thereof further includes VL CDRs 1-3, and VH CRDs 1 and 3 having one or more amino acid substitutions relative to the CDRs of the parent 3E10 antibody (as shown in FIG. 1) or relative to the 3E10-D31N variant (as shown in FIG. 2A).

Similarly, in some embodiments, a 3E10 antibody or variant thereof, or antigen-binding fragment thereof includes VL CDR1 comprising the amino acid sequence of 3E10-VL-CDR1.1 (SEQ ID NO:28) or 3E10-VL-CDR1.2 (SEQ ID NO:29). In some embodiments, the 3E10 antibody or variant thereof, or antigen-binding fragment thereof further includes VL CDRs 2 and 3, and VH CRDs 1-3 according to the parent 3E10 antibody (as shown in FIG. 1). In some embodiments, the 3E10 antibody or variant thereof, or antigen-binding fragment thereof further includes VL CDRs 2 and 3, and VH CRDs 1-3 according to the 3E10-D31N variant (as shown in FIG. 2A). In some embodiments, the 3E10 antibody or variant thereof, or antigen-binding fragment thereof further includes VL CDRs 2 and 3, and VH CRDs 1-3 having one or more amino acid substitutions relative to the CDRs of the parent 3E10 antibody (as shown in FIG. 1) or relative to the 3E10-D31N variant (as shown in FIG. 2A).

Similarly, in some embodiments, a 3E10 antibody or variant thereof, or antigen-binding fragment thereof includes VL CDR2 comprising the amino acid sequence of 3E10-VL-CDR2.1 (SEQ ID NO:30). In some embodiments, the 3E10 antibody or variant thereof, or antigen-binding fragment thereof further includes VL CDRs 1 and 3, and VH CRDs 1-3 according to the parent 3E10 antibody (as shown in FIG. 1). In some embodiments, the 3E10 antibody or variant thereof, or antigen-binding fragment thereof further includes VL CDRs 1 and 3, and VH CRDs 1-3 according to the 3E10-D31N variant (as shown in FIG. 2A). In some embodiments, the 3E10 antibody or variant thereof, or antigen-binding fragment thereof further includes VL CDRs 1 and 3, and VH CRDs 1-3 having one or more amino acid substitutions relative to the CDRs of the parent 3E10 antibody (as shown in FIG. 1) or relative to the 3E10-D31N variant (as shown in FIG. 2A).

While some of the amino acid substitutions described above are fairly conservative substitutions—e.g., an S to T substitution at position 5 of VL CDR1—other substitutions are to amino acids that have vastly different properties—e.g., an M to L substitution at position 14 of VL CDR1, an H to A substitution at position 15 of VL CDR1, and an E to Q substitution at position 6 of VL CDR2. This suggests, without being bound by theory, that at least these positions within the 3E10 CDR framework are tolerant to other amino acid substitutions.

Accordingly, in some embodiments, a 3E10 antibody or variant thereof, or antigen-binding fragment thereof includes VH CDR2 comprising the amino acid sequence of 3E10-VH-CDR2.3 (SEQ ID NO:31). In some embodiments, the 3E10 antibody or variant thereof, or antigen-binding fragment thereof further includes VL CDRs 1-3, and VH CRDs 1 and 3 according to the parent 3E10 antibody (as shown in FIG. 1). In some embodiments, the 3E10 antibody or variant thereof, or antigen-binding fragment thereof further includes VL CDRs 1-3, and VH CRDs 1 and 3 according to the 3E10-D31N variant (as shown in FIG. 2A). In some embodiments, the 3E10 antibody or variant thereof, or antigen-binding fragment thereof further includes VL CDRs 1-3, and VH CRDs 1 and 3 having one or more amino acid substitutions relative to the CDRs of the parent 3E10 antibody (as shown in FIG. 1) or relative to the 3E10-D31N variant (as shown in FIG. 2A), e.g., as described herein.

Similarly, in some embodiments, a 3E10 antibody or variant thereof, or antigen-binding fragment thereof includes VL CDR1 comprising the amino acid sequence of 3E10-VL-CDR1.3 (SEQ ID NO:32). In some embodiments, the 3E10 antibody or variant thereof, or antigen-binding fragment thereof further includes VL CDRs 2 and 3, and VH CRDs 1-3 according to the parent 3E10 antibody (as shown in FIG. 1). In some embodiments, the 3E10 antibody or variant thereof, or antigen-binding fragment thereof further includes VL CDRs 2 and 3, and VH CRDs 1-3 according to the 3E10-D31N variant (as shown in FIG. 2A). In some embodiments, the 3E10 antibody or variant thereof, or antigen-binding fragment thereof further includes VL CDRs 2 and 3, and VH CRDs 1-3 having one or more amino acid substitutions relative to the CDRs of the parent 3E10 antibody (as shown in FIG. 1) or relative to the 3E10-D31N variant (as shown in FIG. 2A), e.g., as described herein.

Similarly, in some embodiments, a 3E10 antibody or variant thereof, or antigen-binding fragment thereof, includes VL CDR2 comprising the amino acid sequence of 3E10-VL-CDR2.2 (SEQ ID NO:33). In some embodiments, the 3E10 antibody or variant thereof, or antigen-binding fragment thereof further includes VL CDRs 1 and 3, and VH CRDs 1-3 according to the parent 3E10 antibody (as shown in FIG. 1). In some embodiments, the 3E10 antibody or variant thereof, or antigen-binding fragment thereof further includes VL CDRs 1 and 3, and VH CRDs 1-3 according to the 3E10-D31N variant (as shown in FIG. 2A). In some embodiments, the 3E10 antibody or variant thereof, or antigen-binding fragment thereof further includes VL CDRs 1 and 3, and VH CRDs 1-3 having one or more amino acid substitutions relative to the CDRs of the parent 3E10 antibody (as shown in FIG. 1) or relative to the 3E10-D31N variant (as shown in FIG. 2A), e.g., as described herein.

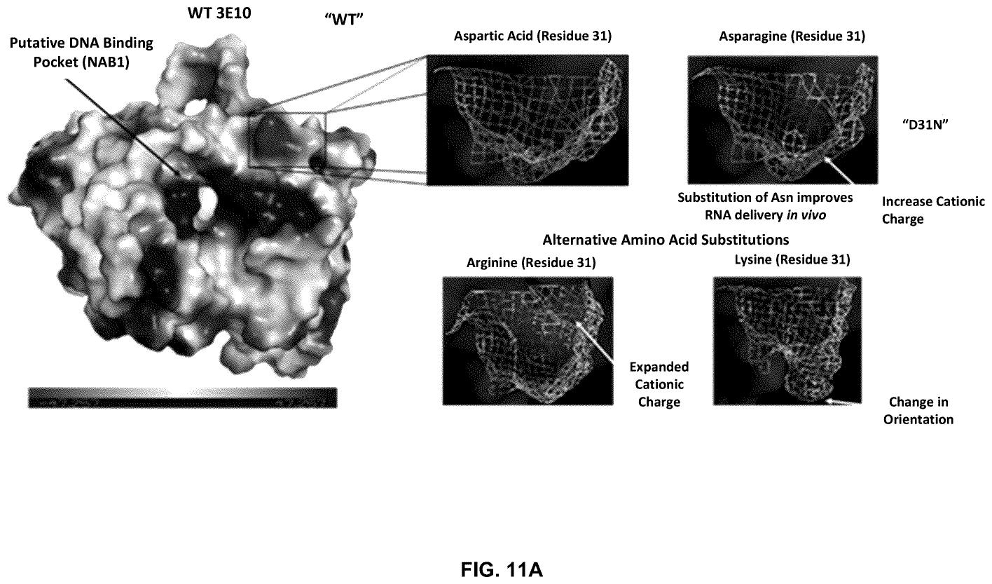

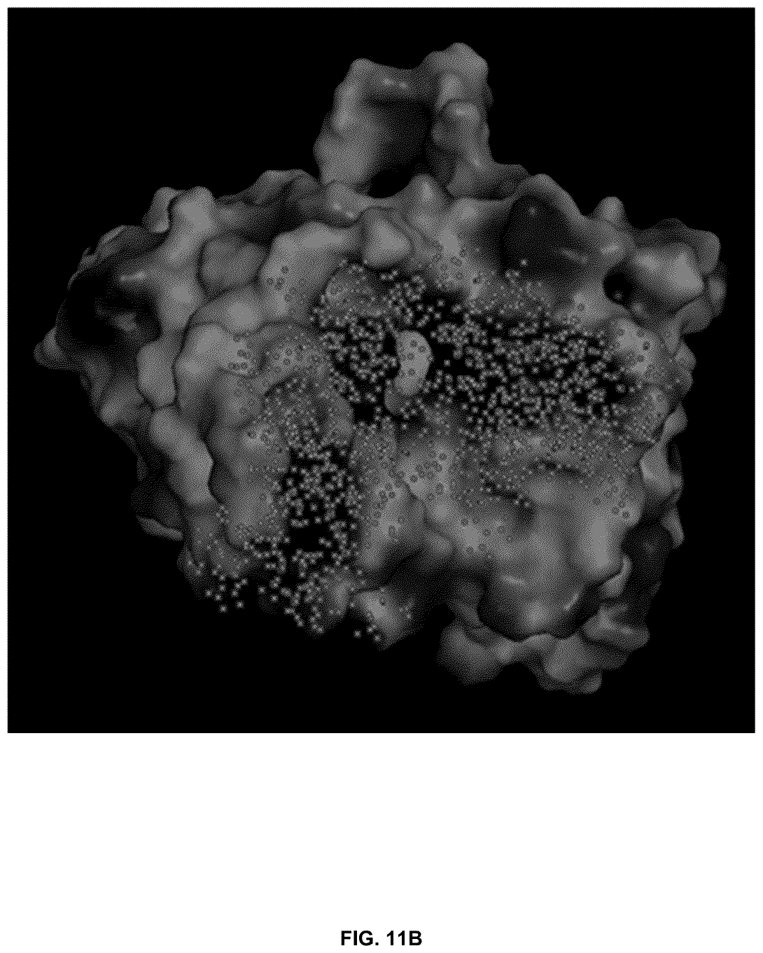

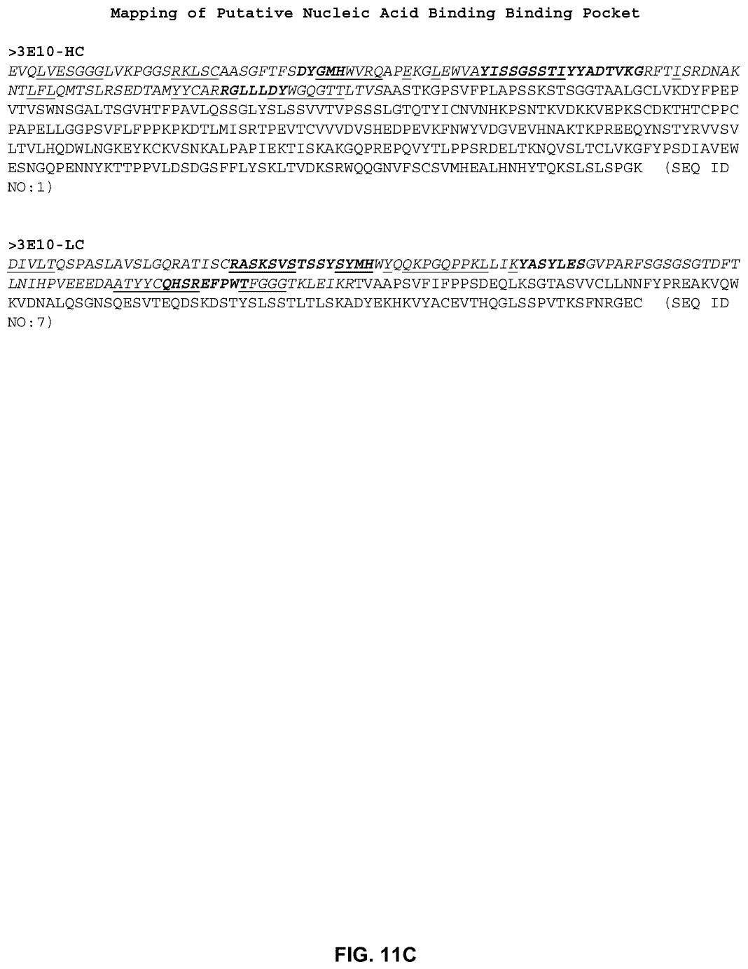

Further, because 3E10 antibodies or variants thereof, or antigen-binding fragments thereof, bind to nucleic acid in a partially sequence-independent manner, and without being bound by theory, it was contemplated that the interaction may be mediated by electrostatic interactions with the nucleotide backbone. To investigate this theory, electrostatic surface potential renderings of a molecular model of a 3E10-scFv construct—the amino acid sequence of which is illustrated in FIG. 11C-were generated, as shown in FIGS. 11A and 11B. These models revealed a putative Nucleic Acid Binding pocket (NAB1) corresponding to a large basic region on the surface of the molecule, as illustrated in FIG. 11A. The position of the non-hydrogen atoms of the amino acids contributing to the putative Nucleic Acid Binding pocket in the model are superposed in FIG. 11B, and the amino acid residues are mapped onto the sequence of the construct in FIG. 11C.

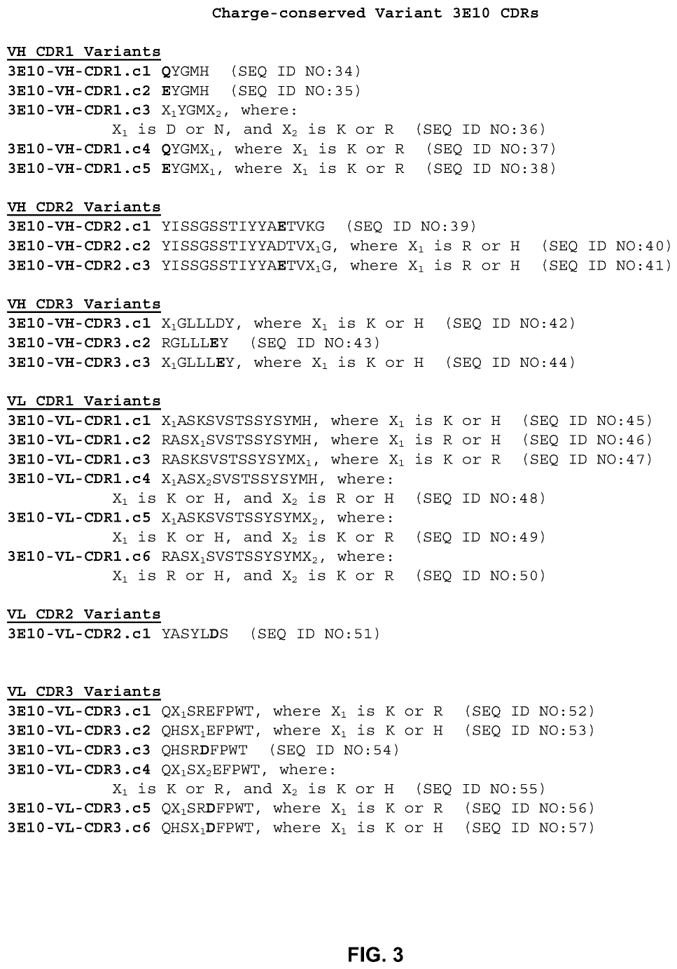

Thus, it is contemplated that amino acid substitutions within the CDRs of a 3E10 antibody or variant thereof, or antigen-binding fragment thereof, as described herein, that maintain the electrostatic character of this putative Nucleic Acid Binding pocket will also retain the nucleic acid binding properties of the construct. Accordingly, in some embodiments, a 3E10 antibody or variant thereof, or antigen-binding fragment thereof, includes one or more amino acid substitution of a first basic amino acid to a second basic amino acid (e.g., K, R, or H). Similarly, in some embodiments, a 3E10 antibody or variant thereof, or antigen-binding fragment thereof, includes one or more amino acid substitution of a first acidic amino acid to a second acidic amino acid (e.g., D or E). Examples of such charge-conserved variant 3E10 CDRs are shown in FIG. 3.

Accordingly, in some embodiments, a 3E10 antibody or variant thereof, or antigen-binding fragment thereof includes VH CDR1 comprising the amino acid sequence of 3E10-VH-CDR1.c1 (SEQ ID NO:34), 3E10-VH-CDR1.c2 (SEQ ID NO:35), 3E10-VH-CDR1.c3 (SEQ ID NO:36), 3E10-VH-CDR1.c4 (SEQ ID NO:37), or 3E10-VH-CDR1.c5 (SEQ ID NO:38). In some embodiments, the 3E10 antibody or variant thereof, or antigen-binding fragment thereof further includes VL CDRs 1-3, and VH CRDs 2 and 3 according to the parent 3E10 antibody (as shown in FIG. 1). In some embodiments, the 3E10 antibody or variant thereof, or antigen-binding fragment thereof further includes VL CDRs 1-3, and VH CRDs 1 and 3 having one or more amino acid substitutions relative to the CDRs of the parent 3E10 antibody (as shown in FIG. 1), e.g., as described herein.

Similarly, in some embodiments, a 3E10 antibody or variant thereof, or antigen-binding fragment thereof includes VH CDR2 comprising the amino acid sequence of 3E10-VH-CDR2.c1 (SEQ ID NO:39), 3E10-VH-CDR2.c2 (SEQ ID NO:40), or 3E10-VH-CDR2.c3 (SEQ ID NO:41). In some embodiments, the 3E10 antibody or variant thereof, or antigen-binding fragment thereof further includes VL CDRs 1-3, and VH CRDs 1 and 3 according to the parent 3E10 antibody (as shown in FIG. 1). In some embodiments, the 3E10 antibody or variant thereof, or antigen-binding fragment thereof further includes VL CDRs 1-3, and VH CRDs 1 and 3 according to the 3E10-D31N variant (as shown in FIG. 2A). In some embodiments, the 3E10 antibody or variant thereof, or antigen-binding fragment thereof further includes VL CDRs 1-3, and VH CRDs 1 and 3 having one or more amino acid substitutions relative to the CDRs of the parent 3E10 antibody (as shown in FIG. 1), e.g., as described herein.

Similarly, in some embodiments, a 3E10 antibody or variant thereof, or antigen-binding fragment thereof includes VH CDR3 comprising the amino acid sequence of 3E10-VH-CDR3.c1 (SEQ ID NO:42), 3E10-VH-CDR3.c2 (SEQ ID NO:43), or 3E10-VH-CDR3.c3 (SEQ ID NO:44). In some embodiments, the 3E10 antibody or variant thereof, or antigen-binding fragment thereof further includes VL CDRs 1-3, and VH CRDs 1 and 2 according to the parent 3E10 antibody (as shown in FIG. 1). In some embodiments, the 3E10 antibody or variant thereof, or antigen-binding fragment thereof further includes VL CDRs 1-3, and VH CRDs 1 and 2 according to the 3E10-D31N variant (as shown in FIG. 2A). In some embodiments, the 3E10 antibody or variant thereof, or antigen-binding fragment thereof further includes VL CDRs 1-3, and VH CRDs 1 and 2 having one or more amino acid substitutions relative to the CDRs of the parent 3E10 antibody (as shown in FIG. 1), e.g., as described herein.

Similarly, in some embodiments, a 3E10 antibody or variant thereof, or antigen-binding fragment thereof includes VL CDR1 comprising the amino acid sequence of 3E10-VL-CDR1.c1 (SEQ ID NO:45), 3E10-VL-CDR1.c2 (SEQ ID NO:46), 3E10-VL-CDR1.c3 (SEQ ID NO:47), 3E10-VL-CDR1.c4 (SEQ ID NO:48), 3E10-VL-CDR1.c5 (SEQ ID NO:49), or 3E10-VL-CDR1.c6 (SEQ ID NO:50). In some embodiments, the 3E10 antibody or variant thereof, or antigen-binding fragment thereof further includes VL CDRs 2 and 3, and VH CRDs 1-3 according to the parent 3E10 antibody (as shown in FIG. 1). In some embodiments, the 3E10 antibody or variant thereof, or antigen-binding fragment thereof further includes VL CDRs 2 and 3, and VH CRDs 1-3 according to the 3E10-D31N variant (as shown in FIG. 2A). In some embodiments, the 3E10 antibody or variant thereof, or antigen-binding fragment thereof further includes VL CDRs 2 and 3, and VH CRDs 1-3 having one or more amino acid substitutions relative to the CDRs of the parent 3E10 antibody (as shown in FIG. 1), e.g., as described herein.

Similarly, in some embodiments, a 3E10 antibody or variant thereof, or antigen-binding fragment thereof includes VL CDR2 comprising the amino acid sequence of 3E10-VL-CDR2.c1 (SEQ ID NO:51). In some embodiments, the 3E10 antibody or variant thereof, or antigen-binding fragment thereof further includes VL CDRs 1 and 3, and VH CRDs 1-3 according to the parent 3E10 antibody (as shown in FIG. 1). In some embodiments, the 3E10 antibody or variant thereof, or antigen-binding fragment thereof further includes VL CDRs 1 and 3, and VH CRDs 1-3 according to the 3E10-D31N variant (as shown in FIG. 2A). In some embodiments, the 3E10 antibody or variant thereof, or antigen-binding fragment thereof further includes VL CDRs 1 and 3, and VH CRDs 1-3 having one or more amino acid substitutions relative to the CDRs of the parent 3E10 antibody (as shown in FIG. 1), e.g., as described herein.

Similarly, in some embodiments, a 3E10 antibody or variant thereof, or antigen-binding fragment thereof includes VL CDR3 comprising the amino acid sequence of 3E10-VL-CDR3.c1 (SEQ ID NO:52), 3E10-VL-CDR3.c2 (SEQ ID NO:53), 3E10-VL-CDR3.c3 (SEQ ID NO:54), 3E10-VL-CDR3.c4 (SEQ ID NO:55), 3E10-VL-CDR3.c5 (SEQ ID NO:56), or 3E10-VL-CDR3.c6 (SEQ ID NO:57). In some embodiments, the 3E10 antibody or variant thereof, or antigen-binding fragment thereof further includes VL CDRs 1 and 2, and VH CRDs 1-3 according to the parent 3E10 antibody (as shown in FIG. 1). In some embodiments, the 3E10 antibody or variant thereof, or antigen-binding fragment thereof further includes VL CDRs 1 and 2, and VH CRDs 1-3 according to the 3E10-D31N variant (as shown in FIG. 2A). In some embodiments, the 3E10 antibody or variant thereof, or antigen-binding fragment thereof further includes VL CDRs 1 and 2, and VH CRDs 1-3 having one or more amino acid substitutions relative to the CDRs of the parent 3E10 antibody (as shown in FIG. 1), e.g., as described herein.

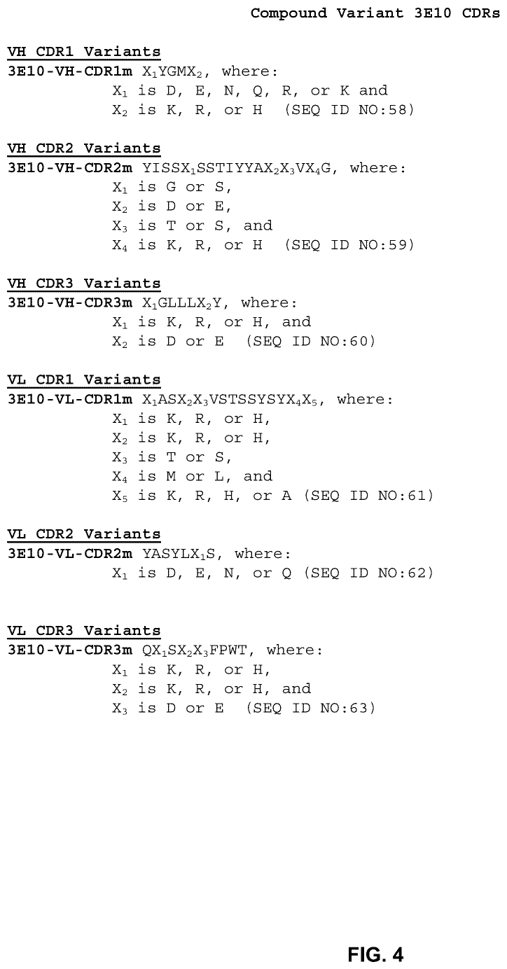

It is also contemplated that a 3E10 antibody or variant thereof, or antigen-binding fragment thereof, as described herein, includes any combination of the 3E10 CDR amino acid substitutions described above. Examples of 3E10 variant CDR sequences that incorporate one or more of the amino acid substitutions described herein are shown in FIG. 4.

Accordingly, in some embodiments, a 3E10 antibody or variant thereof, or antigen-binding fragment thereof includes VH CDR1 comprising the amino acid sequence of 3E10-VH-CDR1m (SEQ ID NO:58). In some embodiments, the 3E10 antibody or variant thereof, or antigen-binding fragment thereof further includes VL CDRs 1-3, and VH CRDs 2 and 3 according to the parent 3E10 antibody (as shown in FIG. 1). In some embodiments, the 3E10 antibody or variant thereof, or antigen-binding fragment thereof further includes VL CDRs 1-3, and VH CRDs 1 and 3 having one or more amino acid substitutions relative to the CDRs of the parent 3E10 antibody (as shown in FIG. 1), e.g., as described herein.

Similarly, in some embodiments, a 3E10 antibody or variant thereof, or antigen-binding fragment thereof includes VH CDR2 comprising the amino acid sequence of 3E10-VH-CDR2m (SEQ ID NO:59). In some embodiments, the 3E10 antibody or variant thereof, or antigen-binding fragment thereof further includes VL CDRs 1-3, and VH CRDs 1 and 3 according to the parent 3E10 antibody (as shown in FIG. 1). In some embodiments, the 3E10 antibody or variant thereof, or antigen-binding fragment thereof further includes VL CDRs 1-3, and VH CRDs 1 and 3 according to the 3E10-D31N variant (as shown in FIG. 2A). In some embodiments, the 3E10 antibody or variant thereof, or antigen-binding fragment thereof further includes VL CDRs 1-3, and VH CRDs 1 and 3 having one or more amino acid substitutions relative to the CDRs of the parent 3E10 antibody (as shown in FIG. 1), e.g., as described herein.

Similarly, in some embodiments, a 3E10 antibody or variant thereof, or antigen-binding fragment thereof includes VH CDR3 comprising the amino acid sequence of 3E10-VH-CDR3m (SEQ ID NO:60). In some embodiments, the 3E10 antibody or variant thereof, or antigen-binding fragment thereof further includes VL CDRs 1-3, and VH CRDs 1 and 2 according to the parent 3E10 antibody (as shown in FIG. 1). In some embodiments, the 3E10 antibody or variant thereof, or antigen-binding fragment thereof further includes VL CDRs 1-3, and VH CRDs 1 and 2 according to the 3E10-D31N variant (as shown in FIG. 2A). In some embodiments, the 3E10 antibody or variant thereof, or antigen-binding fragment thereof further includes VL CDRs 1-3, and VH CRDs 1 and 2 having one or more amino acid substitutions relative to the CDRs of the parent 3E10 antibody (as shown in FIG. 1), e.g., as described herein.

Similarly, in some embodiments, a 3E10 antibody or variant thereof, or antigen-binding fragment thereof includes VL CDR1 comprising the amino acid sequence of 3E10-VL-CDR1m (SEQ ID NO:61). In some embodiments, the 3E10 antibody or variant thereof, or antigen-binding fragment thereof further includes VL CDRs 2 and 3, and VH CRDs 1-3 according to the parent 3E10 antibody (as shown in FIG. 1). In some embodiments, the 3E10 antibody or variant thereof, or antigen-binding fragment thereof further includes VL CDRs 2 and 3, and VH CRDs 1-3 according to the 3E10-D31N variant (as shown in FIG. 2A). In some embodiments, the 3E10 antibody or variant thereof, or antigen-binding fragment thereof further includes VL CDRs 2 and 3, and VH CRDs 1-3 having one or more amino acid substitutions relative to the CDRs of the parent 3E10 antibody (as shown in FIG. 1), e.g., as described herein.

Similarly, in some embodiments, a 3E10 antibody or variant thereof, or antigen-binding fragment thereof includes VL CDR2 comprising the amino acid sequence of 3E10-VL-CDR2m (SEQ ID NO:62). In some embodiments, the 3E10 antibody or variant thereof, or antigen-binding fragment thereof further includes VL CDRs 1 and 3, and VH CRDs 1-3 according to the parent 3E10 antibody (as shown in FIG. 1). In some embodiments, the 3E10 antibody or variant thereof, or antigen-binding fragment thereof further includes VL CDRs 1 and 3, and VH CRDs 1-3 according to the 3E10-D31N variant (as shown in FIG. 2A). In some embodiments, the 3E10 antibody or variant thereof, or antigen-binding fragment thereof further includes VL CDRs 1 and 3, and VH CRDs 1-3 having one or more amino acid substitutions relative to the CDRs of the parent 3E10 antibody (as shown in FIG. 1), e.g., as described herein.

Similarly, in some embodiments, a 3E10 antibody or variant thereof, or antigen-binding fragment thereof includes VL CDR3 comprising the amino acid sequence of 3E10-VL-CDR3m (SEQ ID NO:63). In some embodiments, the 3E10 antibody or variant thereof, or antigen-binding fragment thereof further includes VL CDRs 1 and 2, and VH CRDs 1-3 according to the parent 3E10 antibody (as shown in FIG. 1). In some embodiments, the 3E10 antibody or variant thereof, or antigen-binding fragment thereof further includes VL CDRs 1 and 2, and VH CRDs 1-3 according to the 3E10-D31N variant (as shown in FIG. 2A). In some embodiments, the 3E10 antibody or variant thereof, or antigen-binding fragment thereof further includes VL CDRs 1 and 2, and VH CRDs 1-3 having one or more amino acid substitutions relative to the CDRs of the parent 3E10 antibody (as shown in FIG. 1), e.g., as described herein.

In some embodiments, a 3E10 antibody or variant thereof, or antigen-binding fragment thereof described herein includes a light chain variable region (VL) complementarity determining region (CDR) 1 comprising the amino acid sequence of 3E10-VL-CDR1m (SEQ ID NO:61), a VL CDR2 comprising the amino acid sequence of 3E10-VL-CDR2m (SEQ ID NO:62), a VL CDR3 comprising the amino acid sequence of 3E10-VL-CDR3m (SEQ ID NO:63), a heavy chain variable region (VH) CDR1 comprising the amino acid sequence of 3E10-VH-CDR1m (SEQ ID NO:58), a VH CDR2 comprising the amino acid sequence of 3E10-VH-CDR2m (SEQ ID NO:59), and a VH CDR3 comprising the amino acid sequence of 3E10-VH-CDR3m (SEQ ID NO:60).

In some embodiments, a 3E10 antibody or variant thereof, or antigen-binding fragment thereof described herein refers to CDR sequences having no more than one amino acid substitution relative to the parent 3E10 antibody, shown in FIG. 1, optionally including a D31N amino acid substitution in the VH CDR1. Accordingly, in some embodiments, a 3E10 antibody or variant thereof, or antigen-binding fragment thereof includes a light chain variable region (VL) complementarity determining region (CDR) 1 comprising an amino acid sequence having no more than one amino acid substitution relative to 3E10-VL-CDR1 (SEQ ID NO:9), a VL CDR2 comprising an amino acid sequence having no more than one amino acid substitution relative to 3E10-VL-CDR2 (SEQ ID NO:10), a VL CDR3 comprising an amino acid sequence having no more than one amino acid substitution relative to 3E10-VL-CDR3 (SEQ ID NO:11), a heavy chain variable region (VH) CDR1 comprising an amino acid sequence having no more than one amino acid substitution relative to 3E10-VH-CDR1a (SEQ ID NO:16), a VH CDR2 comprising an amino acid sequence having no more than one amino acid substitution relative to 3E10-VH-CDR2 (SEQ ID NO:4), and a VH CDR3 comprising an amino acid sequence having no more than one amino acid substitution relative to 3E10-VH-CDR3 (SEQ ID NO:5).

In some embodiments, a 3E10 antibody or variant thereof, or antigen-binding fragment thereof described herein refers to CDR sequences having no more than two amino acid substitution relative to the parent 3E10 antibody, shown in FIG. 1, optionally including a D31N amino acid substitution in the VH CDR1. Accordingly, in some embodiments, a 3E10 antibody or variant thereof, or antigen-binding fragment thereof includes a light chain variable region (VL) complementarity determining region (CDR) 1 comprising an amino acid sequence having no more than two amino acid substitutions relative to 3E10-VL-CDR1 (SEQ ID NO:9), a VL CDR2 comprising an amino acid sequence having no more than two amino acid substitutions relative to 3E10-VL-CDR2 (SEQ ID NO:10), a VL CDR3 comprising an amino acid sequence having no more than two amino acid substitutions relative to 3E10-VL-CDR3 (SEQ ID NO:11), a heavy chain variable region (VH) CDR1 comprising an amino acid sequence having no more than two amino acid substitutions relative to 3E10-VH-CDR1a (SEQ ID NO: 16), a VH CDR2 comprising an amino acid sequence having no more than two amino acid substitutions relative to 3E10-VH-CDR2 (SEQ ID NO:4), and a VH CDR3 comprising an amino acid sequence having no more than two amino acid substitutions relative to 3E10-VH-CDR3 (SEQ ID NO:5).

Other variants of a 3E10 antibody or variant thereof, or antigen-binding fragment thereof are also known in the art, as disclosed for example, in Zack, et al., J. Immunol., 157(5):2082-8 (1996). For example, amino acid position 31 of the heavy chain variable region of 3E10 has been determined to be influential in the ability of the antibody and fragments thereof to penetrate nuclei and bind to DNA (bolded in SEQ ID NOs: 13 and 14). A D31N mutation (bolded in SEQ ID NOs: 2 and 13) in CDR1 penetrates nuclei and binds DNA with much greater efficiency than the original antibody (Zack, et al., Immunology and Cell Biology, 72:513-520 (1994), Weisbart, et al., J. Autoimmun., 11, 539-546 (1998); Weisbart, Int. J. Oncol., 25, 1867-1873 (2004)). In some embodiments, the antibody has the D31N substitution.

Although generally referred to herein as “3E10” or “3E10 antibodies,” it will be appreciated that fragments and binding proteins, including antigen-binding fragments, variants, and fusion proteins such as scFv, di-scFv, tr-scFv, and other single chain variable fragments, and other cell-penetrating, nucleic acid transporting molecules disclosed herein are encompassed by the phrase are also expressly provided for use in compositions and methods disclosed herein. Thus, the antibodies and other binding proteins are also referred to herein as cell-penetrating.

In preferred embodiments, the 3E10 antibody is transported into the cytoplasm and/or nucleus of the cells without the aid of a carrier or conjugate. For example, the monoclonal antibody 3E10 and active fragments thereof that are transported in vivo to the nucleus of mammalian cells without cytotoxic effect are disclosed in U.S. Pat. Nos. 4,812,397 and 7,189,396 to Richard Weisbart.

Antibodies useful in the compositions and methods described herein include whole immunoglobulin (i.e., an intact antibody) of any class, fragments thereof, and synthetic proteins containing at least the antigen binding variable domain of an antibody. The variable domains differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen binding site of antibodies. Therefore, the antibodies typically contain at least the CDRs necessary to maintain DNA binding and/or interfere with DNA repair.

The 3E10 antibody is typically a monoclonal 3E10, or a variant, derivative, fragment, fusion, or humanized form thereof that binds the same or different epitope(s) as 3E10.

A deposit according to the terms of the Budapest Treaty of a hybridoma cell line producing monoclonal antibody 3E10 was received on Sep. 6, 2000, and accepted by, American Type Culture Collection (ATCC), 10801 University Blvd., Manassas, VA 20110-2209, USA, and given Patent Deposit Number PTA-2439.

Thus, the antibody may have the same or different epitope specificity as monoclonal antibody 3E10 produced by ATCC No. PTA 2439 hybridoma. The antibody can have the paratope of monoclonal antibody 3E10. The antibody can be a single chain variable fragment of 3E10, or a variant, e.g., a conservative variant thereof. For example, the antibody can be a single chain variable fragment of 3E10 (3E10 Fv), or a variant thereof.

Additionally, or alternatively, the heavy chain complementarity determining regions (CDRs) can be defined according to the IMGT system. The complementarity determining regions (CDRs) as identified by the IMGT system include CDR H1.3 (original sequence): GFTFSDYG (SEQ ID NO:99); CDR H1.4 (with D31N mutation): GFTFSNYG (SEQ ID NO:100); CDR H2.2: ISSGSSTI (SEQ ID NO:101) and variant ISSSSSTI (SEQ ID NO:102); CDR H3.2: ARRGLLLDY (SEQ ID NO:103).

Additionally, or alternatively, the light chain complementarity determining regions (CDRs) can be defined according to the IMGT system. The complementarity determining regions (CDRs) as identified by the IMGT system include CDR L1.2 KSVSTSSYSY (SEQ ID NO:104) and variant KTVSTSSYSY (SEQ ID NO:105); CDR L2.2: YAS (SEQ ID NO:106); CDR L3.2: QHSREFPWT (SEQ ID NO:107).

In some embodiments, the antibody is a humanized antibody. Methods for humanizing non-human antibodies are well known in the art. Generally, a humanized antibody has one or more amino acid residues introduced into it from a source that is non-human. These non-human amino acid residues are often referred to as “import” residues, which are typically taken from an “import” variable domain. Antibody humanization techniques generally involve the use of recombinant DNA technology to manipulate the DNA sequence encoding one or more polypeptide chains of an antibody molecule.

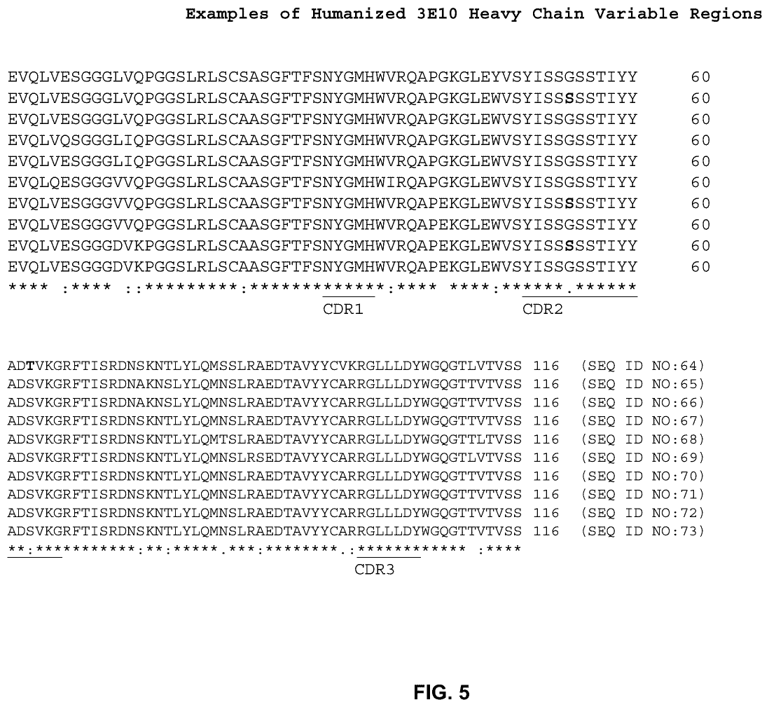

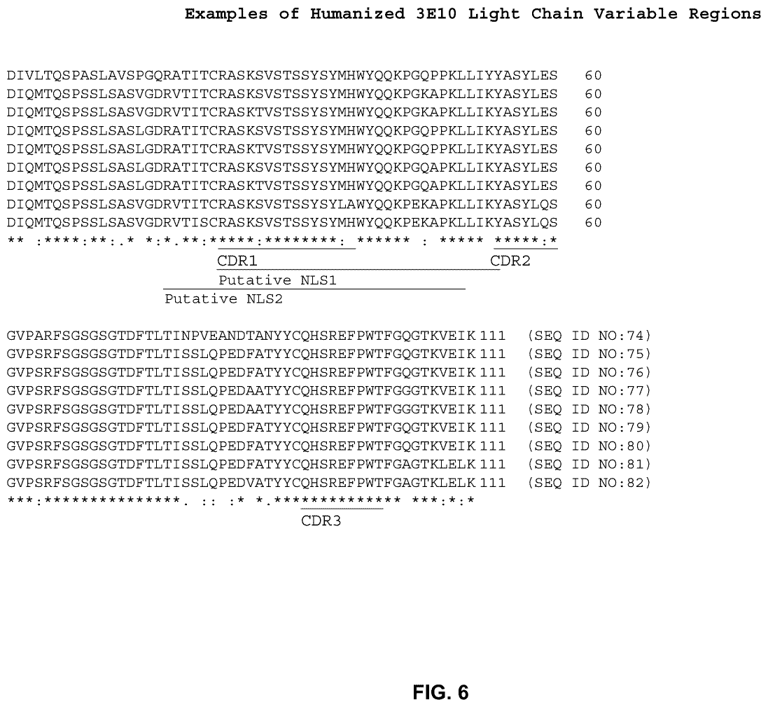

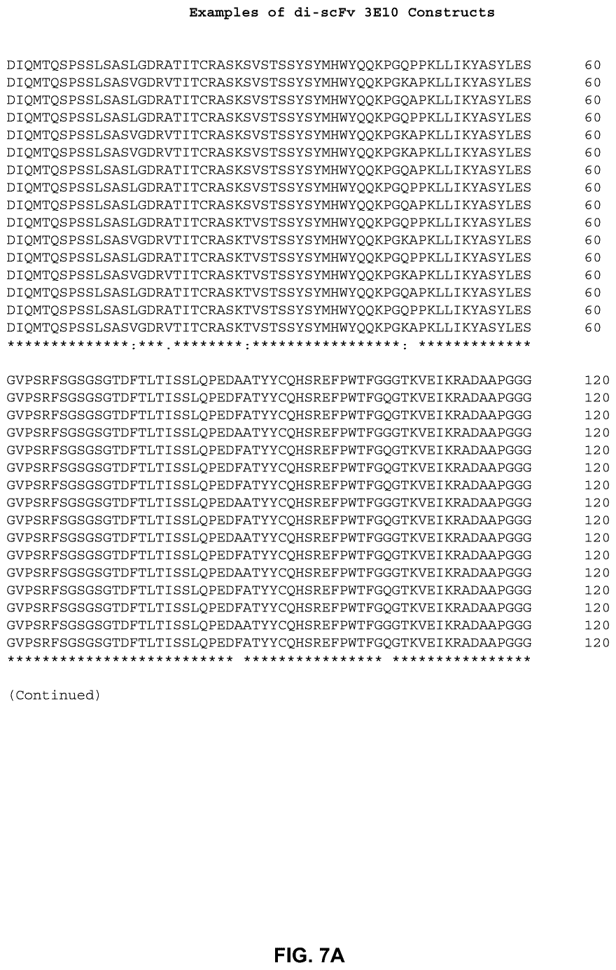

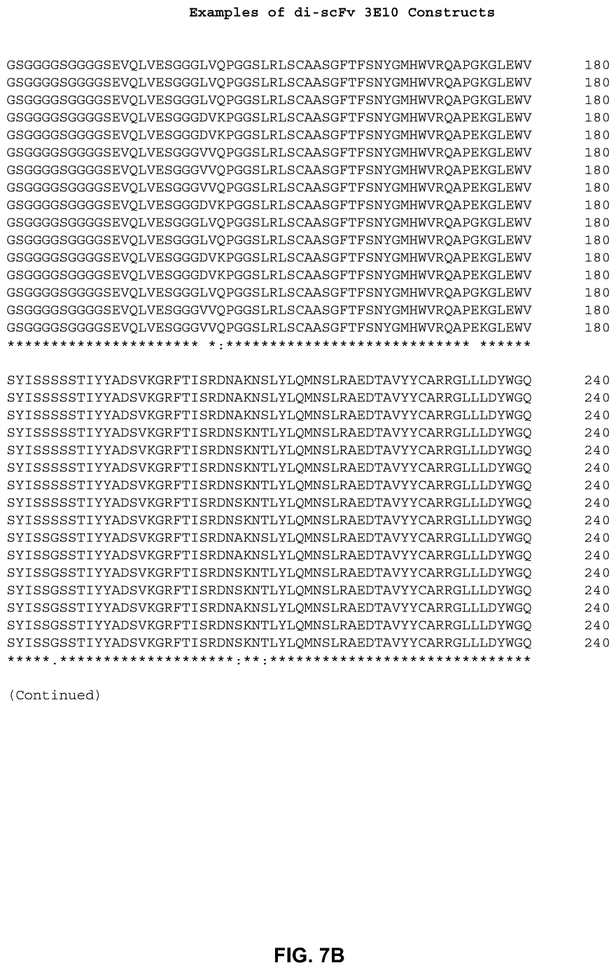

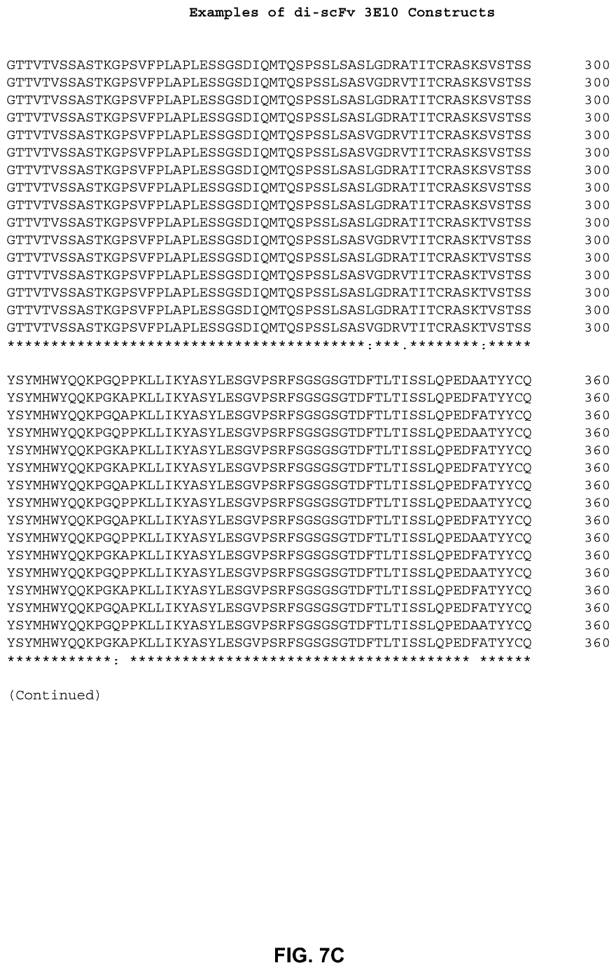

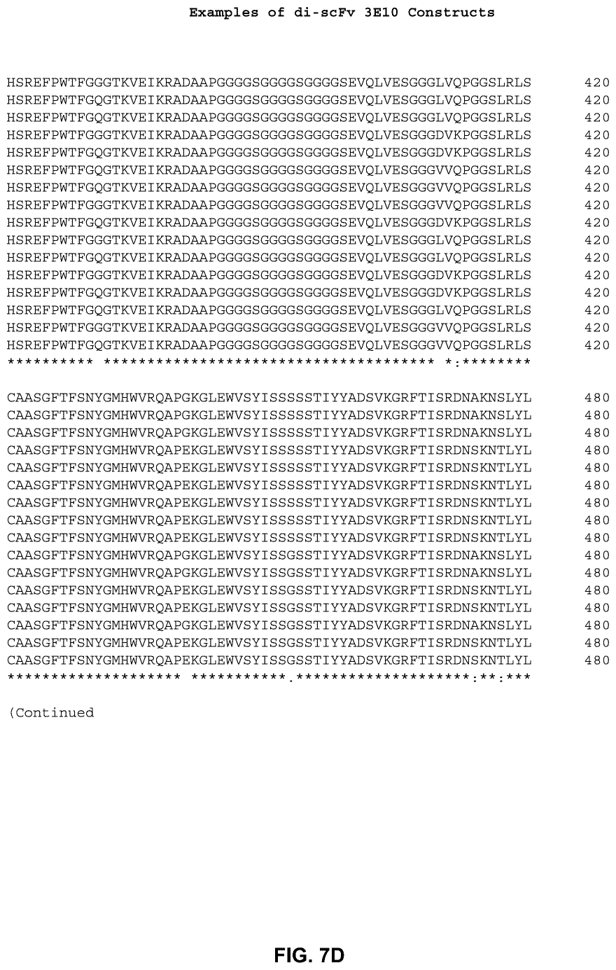

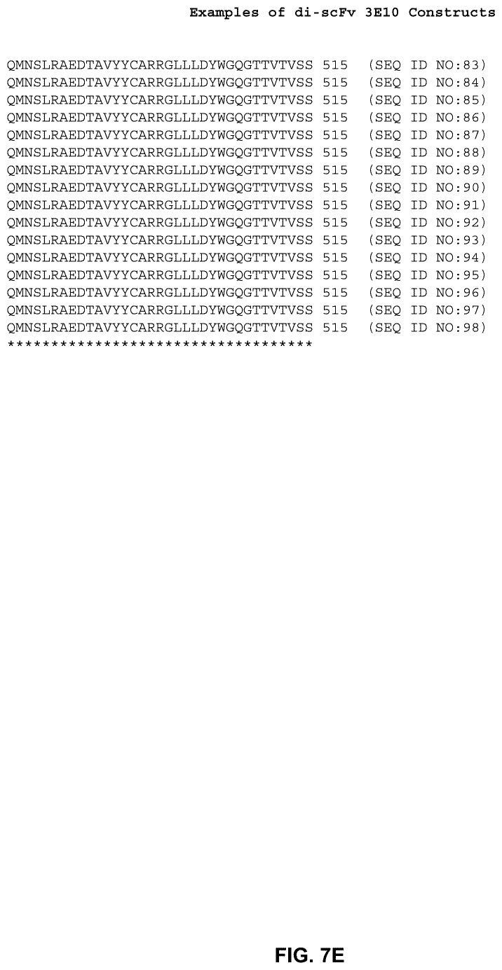

Exemplary 3E10 humanized 3E10 heavy chain variable region (SEQ ID NOs:64-73) and light chain variable region (SEQ ID NOs: 74-82) sequences are discussed in WO 2015/106290 (U.S. Pat. No. 10,221,250), WO 2016/033324 (U.S. Pat. No. 10,501,554), WO 2019/018426 (US 2020/216567), and WO/2019/018428 (US 2020/216568), the disclosures of which are incorporated herein by reference in their entireties for all purposes, and provided in FIGS. 5 and 6, respectively. In some embodiments, the 3E10 antibodies described herein include a heavy chain variable region selected from SEQ ID NOs:64-73 and a light chain variable region selected from SEQ ID NOs:74-82. In some embodiments, the 3E10 antibodies described herein include a heavy chain variable region having at least 99% amino acid identity with a heavy chain variable region selected from SEQ ID NOs:64-73 and a light chain variable region having at least 99% amino acid identity with a light chain variable region selected from SEQ ID NOs:74-82. In some embodiments, the 3E10 antibodies described herein include a heavy chain variable region having at least 98% amino acid identity with a heavy chain variable region selected from SEQ ID NOs:64-73 and a light chain variable region having at least 98% amino acid identity with a light chain variable region selected from SEQ ID NOs: 74-82. In some embodiments, the 3E10 antibodies described herein include a heavy chain variable region having at least 97% amino acid identity with a heavy chain variable region selected from SEQ ID NOs:64-73 and a light chain variable region having at least 97% amino acid identity with a light chain variable region selected from SEQ ID NOs:74-82. In some embodiments, the 3E10 antibodies described herein include a heavy chain variable region having at least 96% amino acid identity with a heavy chain variable region selected from SEQ ID NOs:64-73 and a light chain variable region having at least 96% amino acid identity with a light chain variable region selected from SEQ ID NOs:74-82. In some embodiments, the 3E10 antibodies described herein include a heavy chain variable region having at least 95% amino acid identity with a heavy chain variable region selected from SEQ ID NOs:64-73 and a light chain variable region having at least 95% amino acid identity with a light chain variable region selected from SEQ ID NOs: 74-82. In some embodiments, the 3E10 antibodies described herein include a heavy chain variable region having at least 90%, 91%, 92%, 93%, or 94% amino acid identity with a heavy chain variable region selected from SEQ ID NOs:64-73 and a light chain variable region having at least 90%, 91%, 92%, 93%, or 94% amino acid identity with a light chain variable region selected from SEQ ID NOs:74-82.

The disclosed compositions and methods typically utilize antibodies that maintain the ability to penetrate cells, and optionally nuclei.

The mechanisms of cellular internalization by autoantibodies are diverse. Some are taken into cells through electrostatic interactions or FcR-mediated endocytosis, while others utilize mechanisms based on association with cell surface myosin or calreticulin, followed by endocytosis (Ying-Chyi et al., Eur J Immunol 38, 3178-3190 (2008), Yanase et al., J Clin Invest 100, 25-31 (1997)). 3E10 penetrates cells in an Fc-independent mechanism (as evidenced by the ability of 3E10 fragments lacking an Fc to penetrate cells) but involves presence of the nucleoside transporter ENT2 (Weisbart et al., Sci Rep 5:12022. doi: 10.1038/srep12022. (2015), Zack et al., J Immunol 157, 2082-2088 (1996), Hansen et al., J Biol Chem 282, 20790-20793 (2007)). Thus, in some embodiments, the antibodies utilized in the disclosed compositions and methods are ones that penetrates cells in an Fc-independent mechanism but involves presence of the nucleoside transporter ENT2.

Mutations in 3E10 that interfere with its ability to bind DNA may render the antibody incapable of nuclear penetration. Thus, typically the disclosed variants and humanized forms of the antibody maintain the ability to bind nucleic acids, particularly DNA. In addition, 3E10 scFv has previously been shown capable of penetrating into living cells and nucleic in an ENT2-dependent manner, with efficiency of uptake impaired in ENT2-deficient cells (Hansen, et al., J. Biol. Chem. 282, 20790-20793 (2007)). Thus, in some embodiments, the disclosed variants and humanized forms of the antibody maintain the ability penetrate into cell nuclei in an ENT-dependent, preferably ENT2-dependent manner.

### Nucleic Acid Binding

The disclosed compositions and methods typically utilize antibodies that maintain the ability to bind mRNA.

Example 4 described molecular modeling of 3E10 and additional 3E10 variants. Molecular modeling of 3E10 (Pymol) revealed a putative Nucleic Acid Binding pocket (NAB1) (see, e.g., FIGS. 11A and 11B), and illustrated with underlining in FIG. 11C.

In some embodiments, the disclosed antibodies include some or all of the underlined NAB1 sequences. In some embodiments, the antibodies include a variant sequence that has an altered ability of bind nucleic acids. In some embodiments, the mutations (e.g., substitutions, insertions, and/or deletions) in the NAB1 improve binding of the antibody to nucleic acids such as RNA. In some embodiments, the mutations are conservative substitutions. In some embodiments, the mutations increase the cationic charge of the NAB1 pocket.

As discussed and exemplified herein, mutation of aspartic acid at residue 31 of CDR1 to asparagine increased the cationic charge of this residue and enhanced nucleic acid binding and delivery in vivo (3E10-D31N). Additional exemplary variants include mutation of aspartic acid at residue 31 of CDR1 to arginine (3E10-D31R), which modeling indicates expands cationic charge, or lysine (3E10-D31K) which modeling indicates changes charge orientation. Thus, in some embodiments, the 3E10 binding protein includes a D31R or D31K substitution.

All of the sequences disclosed herein having the residue corresponding to 3E10 D31 or N31, are expressly disclosed with a D31R or D31K or N31R or N31K substitution therein.

Molecular modeling of 3E10 (Pymol) revealed a putative Nucleic Acid Binding pocket (NAB1) (FIGS. 11A-11B). Mutation of aspartic acid at residue 31 of CDR1 to asparagine increased the cationic charge of this residue and enhanced nucleic acid binding and delivery in vivo (3E10-D31N). Mutation of aspartic acid at residue 31 of CDR1 to arginine (3E10-D31R), further expanded the cationic charge while mutation to lysine (3E10-D31K) changed charge orientation (FIG. 11A).

NAB1 amino acids predicted from molecular modeling have been underlined in the heavy and light chain sequences in FIG. 11C. FIG. 11B is an illustration showing molecular modeling of 3E10-scFv (Pymol) with NAB1 amino acid residues illustrated with punctate dots.

### Genetic Myopathies

Gene replacement therapy refers to a number of therapeutic techniques for delivering a functional copy of a gene to a tissue in need of the protein encoded by the gene, including DNA-based gene therapy techniques in which a functional copy of the gene is transcribed within the cell, e.g., with or without being stably integrated into the genome of the subject, gene editing therapies, such as CRISPR/Cas, that repair or replace mutant copies of the gene or specific nucleotides in the host's genome, and mRNA delivery-based approaches in which mRNA encoding the protein are delivered to the cell, eliminating the need to transcribe an exogenous copy of the gene. Researchers have developed, and continue to develop, gene replacement therapies for a diverse set of disorders, most notably genetic disorders and cancers in a subject has one or two mutant or non-functioning copies of the gene, e.g., due to mutations in the gene that cause partial or complete loss-of-function, mutations in an associated regulatory region that down-regulates gene transcription, and/or small genomic deletions.

Myopathies are clinical disorders of the skeletal muscles. These disorders are typically characterized by abnormalities of muscle cell structure and/or metabolism, resulting in various patterns of muscle weakness and dysfunction. There are many types of genetic myopathies, caused by mutations in one or more of a large set of genes. Subjects with genetic myopathies common suffer from muscle weakness, motor delay, respiratory impairment, and bulbar muscle dysfunction. Because the etiology of many different forms of genetic myopathies has been well characterized, gene therapies offer an attractive option for treating these disorders. In fact, clinical trials for such gene therapies have been initiated for several genetic myopathies.

One such disorder for which a gene therapy is being developed is x-linked myotubular myopathy (XLMTM). MTM is a congenital myopathy caused by loss of function mutations in the myotubularin (MTM1) gene that affects 1 in 50,000 live male births. Pierson C R, Ann Transl Med., 3(5):61 (2015), the content of which is incorporated herein by reference. Adeno-associated virus (AAV)-mediated delivery of a gene therapy vector encoding a functional MTM1 gene has shown promise for treating MTM in mice, canine, and human subjects. See, Buj-Bello, Anna et al., Human molecular genetics, (17)14: 2132-43 (2008); Childers M K, Sci Transl Med., 6(220):220ra10 (2014); and Kaiser J., “Boys with a rare muscle disease are breathing on their own, thanks to gene therapy” doi: 10.1126/science.aax9005, the contents of which are incorporated herein by reference. Further, AAV-mediated delivery of a gene therapy vector encoding Myotubularin-related protein 2 (MTMR2), a homologue of the MTM1 gene improves motor activity and muscle strength in MTM1-deficient knock-out mice. Danièle N. et al., J Neuropathol Exp Neurol., 77(4):282-95 (2018), the content of which is incorporated herein by reference.

Similarly, gene therapy is being developed for treating Duchenne muscular dystrophy (DMD). DMD is an x-linked myopathy caused by loss-of-function mutations in the dystrophin (DMD) gene that affects 1 in 3,500-5,000 live male births. Several human clinical trials are ongoing for the treatment of DMD by AAV-mediated delivery of genes encoding smaller, functioning version of the dystrophin protein, sometimes referred to as mini-dystrophin or micro-dystrophin. Duan D., Mol Ther., 26(10):2337-56 (2018), the content of which is incorporated herein by reference. Further examples of skeletal muscle diseases for which clinical trials have been initiated include Becker muscular dystrophy and limb-girdle muscular dystrophy. Braun R. et al., Am J Phys Med Rehabil., 93(11 Suppl 3):S97-S107 (2014), the content of which is incorporated herein by reference.

Accordingly, in one aspect, the present disclosure provides methods for treating a skeletal muscle disease in a subject by delivering a complex of a therapeutic mRNA encoding a skeletal muscle protein and a 3E10 antibody or variant thereof, or antigen-binding fragment thereof, as described herein, to a skeletal muscle of the subject.

Although, in some embodiments, the polypeptide encoded by the mRNA is a wild-type version of the skeletal muscle protein, it will be appreciated that naturally occurring variants or synthetically engineered versions of a skeletal muscle protein may also find use in the compositions and methods described herein. For example, in instances where mRNA therapy is used for enzyme replacement therapy, it is common for the enzyme encoded by the mRNA to be engineered to improve enzymatic activity. Further, in certain instances, where the wild type version of a therapeutic protein is particularly large and/or includes one or more domains that are particularly susceptible to proteolytic degradation, is it common for the protein encoded by a gene therapy vector to be engineered to make the protein smaller and/or to remove susceptible regions that are dispensable for protein function.

In some embodiments, the therapeutic mRNA molecule encodes for a skeletal-muscle protein. Non-limiting examples of skeletal-muscle proteins include nebulin (NEB), skeletal muscle alpha-actin (ACTA), alpha-tropomyosin-3 (TPM3), beta-tropomyosin-2 (TPM2), troponin T1 (TNNT1), cofilin-2 (CFL2), Kelch-repeat-and-BTB-domain-containing-13 (KBTBD13), Kelch-like-family member-40 (KLHL40), Kelch-like protein 4 (KLHL4), Kelch-like-family member 41 (KLHL41), leiomodin-3 (LMOD3), myopalladin (MYPN), ryanodine receptor (RYR1), selenoprotein N (SEPN1), myotubularin (MTM1), dynamin-2 (DNM2), amphiphysin-2 (BIN1), titin (TTN), striated muscle preferentially expressed protein kinase (SPEG), slow-skeletal/beta-cardiac myosin heavy chain (MYH7) cytochrome b, cytochrome c oxidase, thymidine kinase (TK2), polymerase gamma 1 (POLG1), lysosomal enzyme acid alpha-glucosidase (GAA), glycogen-debranching enzyme (AGL), myophosphorylase (PYGM), carnitine transporter OCTN2 (SLC22A5), electron-transfer flavoprotein (ETF), ETF-dehydrogenase (ETFH), adipose triglyceride lipase (PNPLA2), skeletal muscle chloride channel (CIC1), alpha-subunit of the skeletal muscle sodium channel (SCN4A), myotonin-protein kinase (DMPK), zinc finger 9 (ZNF9), dystrophin (DMD), myotilin (MYOT), lamin A/C (LMNA), caveolin 3 (CAV3), DnaJ Heat Shock Protein Family (Hsp40) Member B6 (DNAJB6), desmin (DES), transportin 3 (TNPO3), Heterogeneous nuclear ribonucleoprotein D-like (HNRPDL), calpain 3, dysferlin (DYSF), gamma-sarcoglycan (SGCG), alpha-sarcoglycan (SGCA), beta-sarcoglycan (SGCB), delta-sarcoglycan (SGCD), telethonin (TCAP), E3 ubiquitin-protein ligase TRIM32 (TRIM32), Fukutin-related protein (FKRP), Protein O-mannosyl-transferase 1 (POMT1), anoctamin 5 (ANO5), fukutin, Protein O-mannosyl-transferase 2 (POMT2), O-linked-mannose beta-1,2-N acetylglucosaminyltransferase (POMTnG1), dystroglycan (DAG1), plectin (PLEC1), LGMD2R, Trafficking protein particle complex subunit 11 (TRAPPC11), Mannose-1-phosphate guanyltransferase beta (GMPPB), D-ribitol-5-phosphate cytidylyltransferase (ISPD), alpha-glucosidase, LIM and senescent cell antigen-like-containing domain protein 2 (LIMS2), isoprenoid synthase domain containing (ISPD), Popeye-domain containing 1 (POPDC1), lamina-associated polypeptide 1B (TORIAIP1), O-glucosyltransferase 1 (POGLUT1), Laminin subunit alpha-2 (LAMA2), collagen alpha-1(VI) chain (COL6A1), collagen alpha-2(VI) chain (COL6A2), collagen alpha-3(VI) chain (COL6A3), double homeobox 4 (DUX4), and emerin (EMD).

In some embodiments, the subject has a genetic skeletal muscle disease. For example, in some embodiments, the subject carries a skeletal muscle gene having a partial or complete loss-of-function mutation. Accordingly, in some embodiments, the therapeutic mRNA administered to the subject encodes for a functional copy of a polypeptide corresponding to the mutated gene in the subject. However, in some instances, such as with MTMR2-mediated gene therapy for x-linked myotubular myopathy (described above), the mRNA encodes for a homologue of the protein encoded by the mutant gene in the subject, a protein that has partially redundant function, and/or a protein that functions in a partially-redundant pathway as the protein encoded by the mutant gene in the subject.

In some embodiments, the genetic skeletal muscle disease is a non-dystrophic genetic myopathy. Non-limiting examples of non-dystrophic genetic myopathies include nemaline myopathy, core myopathy (central and multi-minicore), centronuclear myopathy/myotubular myopathy (XLMTM), congenital fiber-type disproportion myopathy, myosin storage myopathy, mitochondrial myopathy, genetic myopathy, Metabolic myopathy (lipid storage disease), congenital myotonia, and paramyotonia congenital. For a review of example non-dystrophic genetic myopathies see, for example, Muscle Cell and Tissue—Current Status of Research Field, Edited by Kunihiro Sakuma, Chapter 6 “Genetic Myopathies” (2018), the content of which is incorporated herein by reference.

In some embodiments, the genetic skeletal muscle disease is a dystrophic genetic myopathy. Non-limiting examples of dystrophic genetic myopathies include a myotonic dystrophy (DM1/DM2), Duchenne muscular dystrophy, Becker muscular dystrophy, autosomal-dominant form of limb-girdle muscular dystrophy (LGMD1), autosomal-recessive form of limb-girdle muscular dystrophy (LGMD2), congenital muscular dystrophy, facioscapulohumeral muscular dystrophy, and Emery-dreifuss muscular dystrophy. For a review of example dystrophic genetic myopathies see, for example, Muscle Cell and Tissue—Current Status of Research Field, Edited by Kunihiro Sakuma, Chapter 6 “Genetic Myopathies” (2018), the content of which is incorporated herein by reference.

Each of the classes of myopathies listed above has been associated with a mutation in one or more skeletal-muscle genes. Examples of the genes found to be associated with particular skeletal muscle diseases are listed in Table 2, below. Accordingly, in some embodiments, a subject with a particular skeletal muscle disease is treated by administration of a 3E10-mRNA complex where the mRNA encodes for a polypeptide corresponding to an associated gene in Table 2. For example, in one embodiment, a mRNA molecule encoding a polypeptide associated with the myotubularin (MTM1) protein is used for the treatment of a type of myotubular myopathy, e.g., x-linked myotubular myopathy (XLMTM).

TABLE 2
Example genes found to be mutated in various skeletal muscle diseases.

| Skeletal Muscle Disease | Mutant Protein (Genes) |
|---|---|
| Nemalin myopathy | Nebulin (NEB = NG_009382.2); Homo sapiens nebulin |
| | (NEB), RefSeqGene (LRG_202) on chromosome 2 |
| | Skeletal muscle alpha-actin (ACTA1 = NG_006672.1); |
| | Homo sapiens actin alpha 1, skeletal muscle (ACTA1), |
| | RefSeqGene (LRG_429) on chromosome 1 |
| | Alpha-tropomyosin-3 (TPM3 = NG_008621.1); Homo |
| | sapiens tropomyosin 3 (TPM3), RefSeqGene (LRG_681) on |
| | chromosome 1 |
| | Beta-tropomyosin-2 (TPM2 = NG_011620.1); Homo sapiens |
| | tropomyosin 2 (TPM2), RefSeqGene (LRG_680) on |
| | chromosome 9 |
| | Troponin T1 (TNNT1 = NG_011829.2); Homo sapiens |
| | troponin T1, slow skeletal type (TNNT1), RefSeqGene |
| | (LRG_679) on chromosome 19 |
| | Cofilin-2 (CFL2 = NG_012740.1); Homo sapiens cofilin 2 |
| | (CFL2), RefSeqGene (LRG_213) on chromosome 14 |
| | Kelch-repeat-and-BTB-domain-containing-13 (KBTBD13 = |
| | NG_021411.1); Homo sapiens kelch repeat and BTB domain |
| | containing 13 (KBTBD13), RefSeqGene (LRG_682) on |
| | chromosome 15 |
| | Kelch-like-family member-40 (KLHL40 = NG_033035.1); |
| | Homo sapiens kelch like family member 40 (KLHL40), |
| | RefSeqGene on chromosome 3 |
| | Kelch-like protein 4 (KLHL4 = NG_012815.1); Homo |
| | sapiens kelch like family member 4 (KLHL4), RefSeqGene on |
| | chromosome X |
| | Kelch-like-family member 41 (KLHL41 = NG_042051.1); |
| | Homo sapiens kelch like family member 41 (KLHL41), |
| | RefSeqGene on chromosome 2 |
| | Leiomodin-3 (LMOD3 = NG_041828.1); Homo sapiens |
| | leiomodin 3 (LMOD3), RefSeqGene on chromosome 3 |
| | Myopalladin (MYPN = NG_032118.1); Homo sapiens |
| | myopalladin (MYPN), RefSeqGene (LRG_410) on |
| | chromosome 10 |
| Core myopathy | Ryanodine receptor channel (RYR1 = NG_008866.1); Homo |
| (Central and multiminicore) | sapiens ryanodine receptor 1 (RYR1), RefSeqGene |
| | (LRG_766) on chromosome 19 |
| | Selenoprotein N (SELENON = NG_009930.1); Homo sapiens |
| | selenoprotein N (SELENON), RefSeqGene (LRG_857) on |
| | chromosome 1 |
| Centronuclear myopathy/ | Myotubularin (MTM1 = NG_008199.1); Homo sapiens |
| Myotubular myopathy | myotubularin 1 (MTM1), RefSeqGene (LRG_839) on |
| (XLMTM) | chromosome X |
| | Dynamin-2 (DNM2 = NG_032118.1); Homo sapiens |
| | myopalladin (MYPN), RefSeqGene (LRG_410) on |
| | chromosome 10 |
| | Amphiphysin-2 (BIN1 = NG_012042.1); Homo sapiens |
| | bridging integrator 1 (BIN1), RefSeqGene (LRG_873) on |
| | chromosome 2 |
| | Ryanodine receptor channel (RYR1 = NG_008866.1); Homo |
| | sapiens ryanodine receptor 1 (RYR1), RefSeqGene |
| | (LRG_766) on chromosome 19 |
| | Titin (TTN = NG_011618.3); Homo sapiens titin (TTN), |
| | RefSeqGene (LRG_391) on chromosome 2 |
| | Striated muscle preferentially expressed protein kinase |
| | (SPEG = NG_051022.1); Homo sapiens striated muscle |
| | enriched protein kinase (SPEG), RefSeqGene on chromosome 2 |
| Congenital fiber-type | Skeletal muscle alpha-actin (ACTA1 = NG_006672.1); |
| disproportion myopathy | Homo sapiens actin alpha 1, skeletal muscle (ACTA1), |
| | RefSeqGene (LRG_429) on chromosome 1 |
| | Alpha-tropomyosin-3 (TPM3 = NG_008621.1); Homo |
| | sapiens tropomyosin 3 (TPM3), RefSeqGene (LRG_681) on |
| | chromosome 1 |
| | Ryanodine receptor channel (RYR1 = NG_008866.1); Homo |
| | sapiens ryanodine receptor 1 (RYR1), RefSeqGene |
| | (LRG_766) on chromosome 19 |
| Myosin storage myopathy | Slow-skeletal/beta-cardiac myosin heavy chain (MYH7 = |
| | NG_007884.1); Homo sapiens myosin heavy chain 7 (MYH7), |
| | RefSeqGene (LRG_384) on chromosome 14 |
| Mitochondrial myopathy | Cytochrome b, cytochrome c oxidase, thymidine kinase |
| | (TK2 = NG_016862.1); Homo sapiens thymidine kinase 2 |
| | (TK2), RefSeqGene on chromosome 16; nuclear gene for |
| | mitochondrial product |
| | Polymerase gamma 1 (POLG = NG_008218.2); Homo |
| | sapiens DNA polymerase gamma, catalytic subunit (POLG), |
| | RefSeqGene (LRG_765) on chromosome 15 |
| Metabolic myopathy | Lysosomal enzyme acid alpha-glucosidase (GAA = |
| (glycogen storage disease) | NG_009822.1); Homo sapiens alpha glucosidase (GAA), |
| (Pompe Disease) | RefSeqGene (LRG_673) on chromosome 17 |
| | Glycogen-debranching enzyme (AGL = NG_012865.1); |
| | Homo sapiens amylo-alpha-1, 6-glucosidase, 4-alpha- |
| | glucanotransferase (AGL), RefSeqGene on chromosome 1 |
| | Myophosphorylase (PYGM = NG_013018.1); Homo sapiens |
| | glycogen phosphorylase, muscle associated (PYGM), |
| | RefSeqGene on chromosome 11 |
| Metabolic myopathy | Carnitine transporter OCTN2 (SLC22A5 = NG_008982.2); |
| (lipid storage disease) | Homo sapiens solute carrier family 22 member 5 (SLC22A5), |
| | RefSeqGene on chromosome 5 |
| | Electron-transfer flavoprotein (ETF) |
| | ETF-dehydrogenase (ETFH) |
| | Adipose triglyceride lipase (PNPLA2 = NG_023394.1); |
| | Homo sapiens patatin like phospholipase domain containing 2 |
| | (PNPLA2), RefSeqGene on chromosome 11 |
| Congenital myotonia | Skeletal muscle chloride channel (CLCN1 = NG_009815.2); |
| | Homo sapiens chloride voltage-gated channel 1 (CLCN1), |
| | RefSeqGene on chromosome 7 |
| Paramyotonia congenita | Alpha-subunit of the skeletal muscle sodium channel |
| | (SCN4A = NG_011699.1); Homo sapiens sodium voltage- |
| | gated channel alpha subunit 4 (SCN4A), RefSeqGene on |
| | chromosome 17 |
| Myotonic dystrophy | Myotonin-protein kinase (DMPK = NG_009784.1); Homo |
| (DM1/DM2) | sapiens DM1 protein kinase (DMPK), RefSeqGene on |
| | chromosome 19 |
| | Zinc finger 9 (CNBP = NG_011902.1); Homo sapiens CCHC- |
| | type zinc finger nucleic acid binding protein (CNBP), |
| | RefSeqGene on chromosome 3 |
| Duchenne and Becker | Dystrophin (DMD = NG_012232.1; Homo sapiens dystrophin |
| muscular dystrophy | (DMD), RefSeqGene (LRG_199) on chromosome X) |
| Autosomal-dominant form of | Myotilin (MYOT = NG_008894.1); Homo sapiens myotilin |
| limb-girdle muscular | (MYOT), RefSeqGene (LRG_201) on chromosome 5 |
| dystrophy (LGMD1) | Lamin A/C (LMNA = NG_008692.2); Homo sapiens lamin |
| | A/C (LMNA), RefSeqGene (LRG_254) on chromosome 1 |
| | Caveolin 3 (CAV3 = NG_008797.2); Homo sapiens caveolin |
| | 3 (CAV3), RefSeqGene (LRG_329) on chromosome 3 |
| | DnaJ Heat Shock Protein Family (Hsp40) Member B6 |
| | (DNAJB6 = NG_032573.1); Homo sapiens DnaJ heat shock |
| | protein family (Hsp40) member B6 (DNAJB6), RefSeqGene |
| | on chromosome 7 |
| | Desmin (DES = NG_008043.1); Homo sapiens desmin (DES), |
| | RefSeqGene (LRG_380) on chromosome 2 |
| | Transportin 3 (TNPO3 = NG_023428.1); Homo sapiens |
| | transportin 3 (TNPO3), RefSeqGene on chromosome 7 |
| | Heterogeneous nuclear ribonucleoprotein D-like |
| | (HNRNPDL = NG_029681.1); Homo sapiens heterogeneous |
| | nuclear ribonucleoprotein D like (HNRNPDL), RefSeqGene |
| | on chromosome 4 |
| Autosomal-recessive form of | Calpain 3 (CAPN3 = NG_008660.1); Homo sapiens calpain 3 |
| limb-girdle muscular | (CAPN3), RefSeqGene (LRG_849) on chromosome 15 |
| dystrophy (LGMD2) | Dysferlin (DYSF = NG_008694.1); Homo sapiens dysferlin |
| | (DYSF), RefSeqGene (LRG_845) on chromosome 2 |
| | Gamma-sarcoglycan (SGCG = NG_008759.1); Homo |
| | sapiens sarcoglycan gamma (SGCG), RefSeqGene (LRG_207) |
| | on chromosome 13 |
| | Alpha-sarcoglycan (SGCA = NG_008889.1); Homo sapiens |
| | sarcoglycan alpha (SGCA), RefSeqGene (LRG_203) on |
| | chromosome 17 |
| | Beta-sarcoglycan (SGCB = NG_008891.1); Homo sapiens |
| | sarcoglycan beta (SGCB), RefSeqGene (LRG_204) on |
| | chromosome 4 |
| | Delta-sarcoglycan (SGCD = NG_008693.2); Homo sapiens |
| | sarcoglycan delta (SGCD), RefSeqGene (LRG_205) on |
| | chromosome 5 |
| | Telethonin (TCAP = NG_008892.1); Homo sapiens titin-cap |
| | (TCAP), RefSeqGene (LRG_210) on chromosome 17 |
| | E3 ubiquitin-protein ligase TRIM32 (TRIM32 = |
| | NG_011619.1); Homo sapiens tripartite motif containing 32 |
| | (TRIM32), RefSeqGene (LRG_211) on chromosome 9 |
| | Fukutin-related protein (FKRP = NG_008898.2); Homo |
| | sapiens fukutin related protein (FKRP), RefSeqGene |
| | (LRG_761) on chromosome 19 |
| | Protein O-mannosyl-transferase 1 (POMT1 = LC030233.1); |
| | Homo sapiens POMT1 mRNA, complete cds, contains 9-bp |
| | deletion |
| | Fukutin (FKTN = NG_008754.1); Homo sapiens fukutin |
| | (FKTN), RefSeqGene (LRG_434) on chromosome 9 |
| | Protein O-mannosyl-transferase 2 (POMT2 = |
| | NG_008897.1); Homo sapiens protein O-mannosyltransferase |
| | 2 (POMT2), RefSeqGene (LRG_844) on chromosome 14 |
| | O-linked-mannose beta-1,2-N |
| | acetylglucosaminyltransferase (POMGNT1 = NG_009205.3; |
| | Homo sapiens protein O-linked mannose N- |
| | acetylglucosaminyltransferase 1 (beta 1,2-) (POMGNT1), |
| | RefSeqGene (LRG_701) on chromosome 1) |
| | Dystroglycan (DAG1 = NG_013230.4); Homo sapiens |
| | dystroglycan 1 (DAG1), RefSeqGene (LRG_854) on |
| | chromosome 3 |
| | Titin (TTN = NG_011618.3); Homo sapiens titin (TTN), |
| | RefSeqGene (LRG_391) on chromosome 2 |
| | Anoctamin 5 (ANO5 = NG_015844.1); Homo sapiens |
| | anoctamin 5 (ANO5), RefSeqGene (LRG_868) on |
| | chromosome 11 |
| | Plectin (PLEC = NG_012492.1); Homo sapiens plectin |
| | (PLEC), RefSeqGene on chromosome 8 |
| | Desmin (DES = NG_008043.1); Homo sapiens desmin (DES), |
| | RefSeqGene (LRG_380) on chromosome 2 |
| | Trafficking protein particle complex subunit 11 |
| | (TRAPPC11 = NG_033102.1); Homo sapiens trafficking |
| | protein particle complex subunit 11 (TRAPPC11), |
| | RefSeqGene on chromosome 4 |
| | Mannose-1-phosphate guanyltransferase beta (GMPPB = |
| | NG_033731.2); Homo sapiens GDP-mannose |
| | pyrophosphorylase B (GMPPB), RefSeqGene on chromosome 3 |
| | D-ribitol-5-phosphate cytidylyltransferase (ISPD) |
| | lysosomal enzyme acid alpha-glucosidase (GAA = |
| | NG_009822.1); Homo sapiens alpha glucosidase (GAA), |
| | RefSeqGene (LRG_673) on chromosome 17 |
| | LIM and senescent cell antigen-like-containing domain |
| | protein 2 (LIMS2 = NG_042235.1); Homo sapiens LIM zinc |
| | finger domain containing 2 (LIMS2), RefSeqGene on |
| | chromosome 2 |
| | Popeye-domain containing 1 (BVES = NG_046732.1); |
| | Homo sapiens blood vessel epicardial substance (BVES), |
| | RefSeqGene on chromosome 6 |
| | Lamina-associated polypeptide 1B (TOR1AIP1 = |
| | NG_042316.1); Homo sapiens torsin 1A interacting protein 1 |
| | (TOR1AIP1), RefSeqGene on chromosome 1 |
| | O-glucosyltransferase 1 (POGLUT1 = NG_034115.1); Homo |
| | sapiens protein O-glucosyltransferase 1 (POGLUT1), |
| | RefSeqGene on chromosome 3 |
| Congenital muscular | Laminin subunit alpha-2 (LAMA2 = NG_008678.1); Homo |
| dystrophies | sapiens laminin subunit alpha 2 (LAMA2), RefSeqGene |
| | (LRG_409) on chromosome 6 |
| | Collagen alpha-1 (VI) chain (COL6A1 = NG_008674.1); |
| | Homo sapiens collagen type VI alpha 1 chain (COL6A1), |
| | RefSeqGene (LRG_475) on chromosome 21 |
| | Collagen alpha-2 (VI) chain (COL6A2 = NG_008675.1); |
| | Homo sapiens collagen type VI alpha 2 chain (COL6A2), |
| | RefSeqGene (LRG_476) on chromosome 21 |
| | Collagen alpha-3 (VI) chain (COL6A3 = NG_008676.1); |
| | Homo sapiens collagen type VI alpha 3 chain (COL6A3), |
| | RefSeqGene (LRG_473) on chromosome 2 |
| | Protein O-mannosyl-transferase 1 (POMT1 = |
| | NG_008896.2); Homo sapiens protein O-mannosyltransferase |
| | 1 (POMT1), RefSeqGene (LRG_842) on chromosome 9 |
| | Protein O-mannosyl-transferase 2 (POMT2 = |
| | NG_008897.1); Homo sapiens protein O-mannosyltransferase |
| | 2 (POMT2), RefSeqGene (LRG 844) on chromosome 14 |
| Facioscapulohumeral | Double homeobox 4 (DUX4 = NG_034189.3); Homo sapiens |
| muscular dystrophy | double homeobox 4 (DUX4), RefSeqGene (LRG_1075) on |
| | chromosome 4 |
| Emery-dreifuss muscular | Emerin (EMD = NG_008677.1); Homo sapiens emerin |
| dystrophy | (EMD), RefSeqGene (LRG_745) on chromosome X |
| | Lamin A/C (LMNA = NG_008692.2); Homo sapiens lamin |
| | A/C (LMNA), RefSeqGene (LRG_254) on chromosome 1 |

### Compositions for Treating Genetic Myopathies

In one aspect, the present disclosure provides pharmaceutical compositions including a complex formed between a therapeutic mRNA polynucleotide encoding a skeletal muscle polypeptide, as described herein, and a 3E10 antibody or variant thereof, or antigen-binding fragment thereof, as described herein.

In some embodiments, a pharmaceutical composition described herein has a molar ratio of 3E10 antibody or variant thereof, or antigen-binding fragment thereof to therapeutic mRNA of at least 2:1. As reported in Examples 6 and 8, the use of molar ratios of 3E10 antibody or variant thereof, or antigen-binding fragment thereof to mRNAs molecules in the compositions described herein protects the mRNA molecule from RNA degradation.

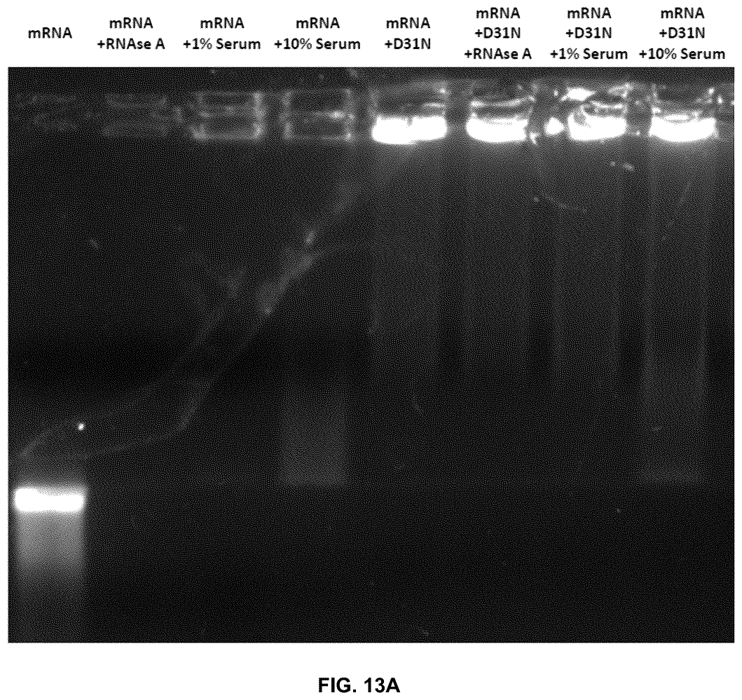

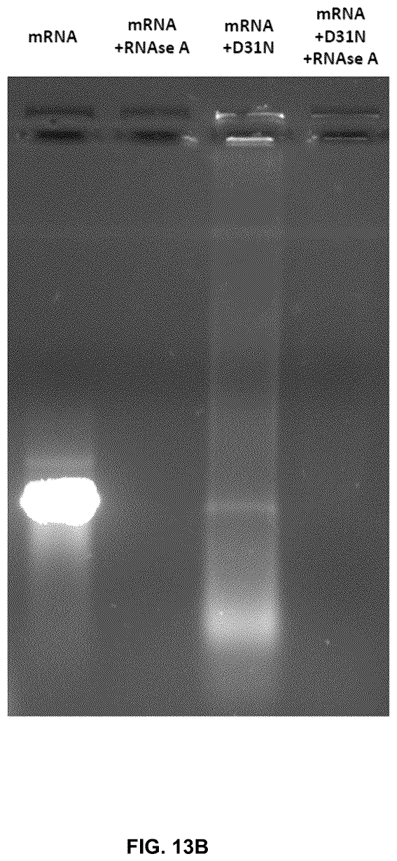

Further, as illustrated in FIGS. 13A and 13B, while parental 3E10 antibodies protected mRNA from RNAse A-mediated RNA degradation at molar ratios of 2:1 and 20:1, the protection afforded by the 20:1 molar ratio exceeded the protection afforded at 2:1. Accordingly, in some embodiments, a pharmaceutical composition described herein has a molar ratio of 3E10 antibody or variant thereof, or antigen-binding fragment thereof to therapeutic polynucleotide that is at least about 2:1. In some embodiments, a pharmaceutical composition described herein has a molar ratio of 3E10 antibody or variant thereof, or antigen-binding fragment thereof to therapeutic polynucleotide that is at least about 5:1. In some embodiments, a pharmaceutical composition described herein has a molar ratio of 3E10 antibody or variant thereof, or antigen-binding fragment thereof to therapeutic polynucleotide that is at least about 7.5:1. In some embodiments, a pharmaceutical composition described herein has a molar ratio of 3E10 antibody or variant thereof, or antigen-binding fragment thereof to therapeutic polynucleotide that is at least about 10:1. In some embodiments, a pharmaceutical composition described herein has a molar ratio of 3E10 antibody or variant thereof, or antigen-binding fragment thereof to therapeutic polynucleotide that is at least about 15:1. In some embodiments, a pharmaceutical composition described herein has a molar ratio of 3E10 antibody or variant thereof, or antigen-binding fragment thereof to therapeutic polynucleotide that is at least about 20:1. In some embodiments, a pharmaceutical composition described herein has a molar ratio of 3E10 antibody or variant thereof, or antigen-binding fragment thereof to therapeutic polynucleotide that is at least about 25:1. In some embodiments, a pharmaceutical composition described herein has a molar ratio of 3E10 antibody or variant thereof, or antigen-binding fragment thereof to therapeutic polynucleotide that is at least about 30:1. In some embodiments, a pharmaceutical composition described herein has a molar ratio of 3E10 antibody or variant thereof, or antigen-binding fragment thereof to therapeutic polynucleotide that is at least about 40:1. In some embodiments, a pharmaceutical composition described herein has a molar ratio of 3E10 antibody or variant thereof, or antigen-binding fragment thereof to therapeutic polynucleotide that is at least about 50:1.

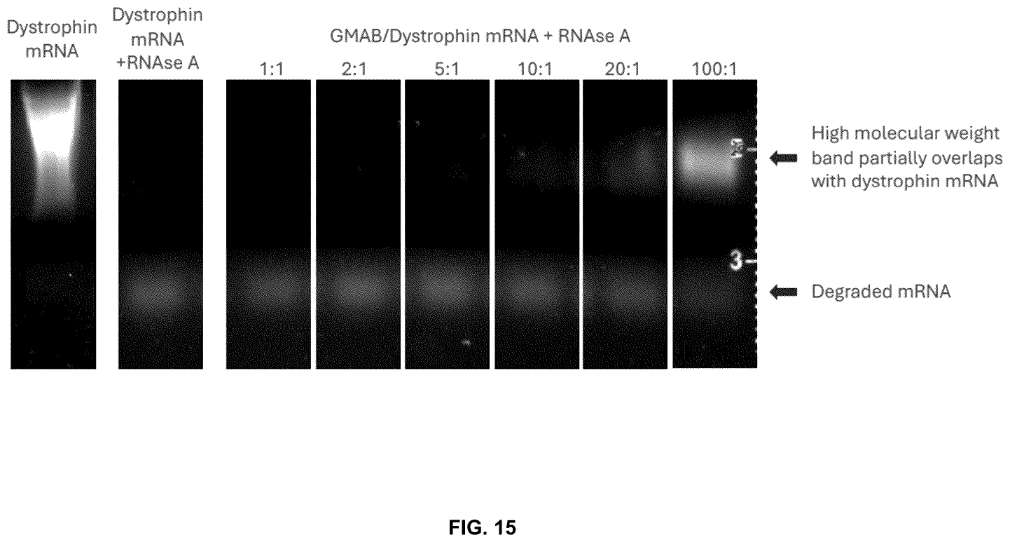

Further, as shown in FIG. 15, the use of higher stoichiometric ratios better protect longer polynucleotides from degradation. Accordingly, in some embodiments, a pharmaceutical composition described herein has a molar ratio of 3E10 antibody or variant thereof, or antigen-binding fragment thereof to therapeutic polynucleotide that is at least about 50:1. In some embodiments, a pharmaceutical composition described herein has a molar ratio of 3E10 antibody or variant thereof, or antigen-binding fragment thereof to therapeutic polynucleotide that is at least about 75:1. In some embodiments, a pharmaceutical composition described herein has a molar ratio of 3E10 antibody or variant thereof, or antigen-binding fragment thereof to therapeutic polynucleotide that is at least about 100:1. In some embodiments, a pharmaceutical composition described herein has a molar ratio of 3E10 antibody or variant thereof, or antigen-binding fragment thereof to therapeutic polynucleotide that is at least about 125:1. In some embodiments, a pharmaceutical composition described herein has a molar ratio of 3E10 antibody or variant thereof, or antigen-binding fragment thereof to therapeutic polynucleotide that is at least about 150:1. In some embodiments, a pharmaceutical composition described herein has a molar ratio of 3E10 antibody or variant thereof, or antigen-binding fragment thereof to therapeutic polynucleotide that is at least about 200:1. In some embodiments, a longer polynucleotide is at least 1000 nucleotides in length, e.g., 1000 nucleotides for a single-stranded polynucleotide or 1000 base pairs for a double-stranded polynucleotide. In some embodiments, a longer polynucleotide is at least 1500 nucleotides in length. In some embodiments, a longer polynucleotide is at least 2000 nucleotides in length. In some embodiments, a longer polynucleotide is at least 2500 nucleotides in length. In some embodiments, a longer polynucleotide is at least 3000 nucleotides in length. In some embodiments, a longer polynucleotide is at least 4000 nucleotides in length. In some embodiments, a longer polynucleotide is at least 5000 nucleotides in length. In some embodiments, a longer polynucleotide is at least 7500 nucleotides in length. In some embodiments, a longer polynucleotide is at least 10,000 nucleotides in length.

In some embodiments, a pharmaceutical composition described herein has a molar ratio of 3E10 antibody or variant thereof, or antigen-binding fragment thereof to therapeutic polynucleotide that is at least about 3:1, at least about 4:1, at least about 5:1, at least about 6:1, at least about 7:1, at least about 8:1, at least about 9:1, at least about 10:1, at least about 11:1, at least about 12:1, at least about 13:1, at least about 14:1, at least about 15:1, at least about 16:1, at least about 17:1, at least about 18:1, at least about 19:1, at least about 20:1, at least about 21:1, at least about 22:1, at least about 23:1, at least about 24:1, at least about 25:1, at least about 26:1, at least about 27:1, at least about 28:1, at least about 29:1, at least about 30:1, at least about 31:1, at least about 32:1, at least about 33:1, at least about 34:1, at least about 35:1, at least about 36:1, at least about 37:1, at least about 38:1, at least about 39:1, at least about 40:1, at least about 41:1, at least about 42:1, at least about 43:1, at least about 44:1, at least about 45:1, at least about 50:1, at least about 55:1, at least about 60:1, at least about 70:1, at least about 75:1, at least about 80:1, at least about 85:1, at least about 90:1, at least about 95:1, at least about 100:1, at least about 110:1, at least about 120:1, at least about 125:1, at least about 130:1, at least about 140:1, at least about 150:1, at least about 160:1, at least about 170:1, at least about 175:1, at least about 180:1, at least about 190:1, at least about 200:1, or greater.

In some embodiments, a pharmaceutical composition described herein has a molar ratio of 3E10 antibody or variant thereof, or antigen-binding fragment thereof to therapeutic polynucleotide that is at least 2:1. In some embodiments, a pharmaceutical composition described herein has a molar ratio of 3E10 antibody or variant thereof, or antigen-binding fragment thereof to therapeutic polynucleotide that is at least 5:1. In some embodiments, a pharmaceutical composition described herein has a molar ratio of 3E10 antibody or variant thereof, or antigen-binding fragment thereof to therapeutic polynucleotide that is at least 7.5:1. In some embodiments, a pharmaceutical composition described herein has a molar ratio of 3E10 antibody or variant thereof, or antigen-binding fragment thereof to therapeutic polynucleotide that is at least 10:1. In some embodiments, a pharmaceutical composition described herein has a molar ratio of 3E10 antibody or variant thereof, or antigen-binding fragment thereof to therapeutic polynucleotide that is at least 15:1. In some embodiments, a pharmaceutical composition described herein has a molar ratio of 3E10 antibody or variant thereof, or antigen-binding fragment thereof to therapeutic polynucleotide that is at least 20:1. In some embodiments, a pharmaceutical composition described herein has a molar ratio of 3E10 antibody or variant thereof, or antigen-binding fragment thereof to therapeutic polynucleotide that is at least 25:1. In some embodiments, a pharmaceutical composition described herein has a molar ratio of 3E10 antibody or variant thereof, or antigen-binding fragment thereof to therapeutic polynucleotide that is at least 30:1. In some embodiments, a pharmaceutical composition described herein has a molar ratio of 3E10 antibody or variant thereof, or antigen-binding fragment thereof to therapeutic polynucleotide that is at least 40:1. In some embodiments, a pharmaceutical composition described herein has a molar ratio of 3E10 antibody or variant thereof, or antigen-binding fragment thereof to therapeutic polynucleotide that is at least 50:1. In some embodiments, a pharmaceutical composition described herein has a molar ratio of 3E10 antibody or variant thereof, or antigen-binding fragment thereof to therapeutic polynucleotide that is at least 75:1. In some embodiments, a pharmaceutical composition described herein has a molar ratio of 3E10 antibody or variant thereof, or antigen-binding fragment thereof to therapeutic polynucleotide that is at least 100:1. In some embodiments, a pharmaceutical composition described herein has a molar ratio of 3E10 antibody or variant thereof, or antigen-binding fragment thereof to therapeutic polynucleotide that is at least 125:1. In some embodiments, a pharmaceutical composition described herein has a molar ratio of 3E10 antibody or variant thereof, or antigen-binding fragment thereof to therapeutic polynucleotide that is at least 150:1. In some embodiments, a pharmaceutical composition described herein has a molar ratio of 3E10 antibody or variant thereof, or antigen-binding fragment thereof to therapeutic polynucleotide that is at least 200:1.

In some embodiments, a pharmaceutical composition described herein has a molar ratio of 3E10 antibody or variant thereof, or antigen-binding fragment thereof to therapeutic polynucleotide that is at least 3:1, at least 4:1, at least 5:1, at least 6:1, at least 7:1, at least 8:1, at least 9:1, at least 10:1, at least 11:1, at least 12:1, at least 13:1, at least 14:1, at least 15:1, at least 16:1, at least 17:1, at least 18:1, at least 19:1, at least 20:1, at least 21:1, at least 22:1, at least 23:1, at least 24:1, at least 25:1, at least 26:1, at least 27:1, at least 28:1, at least 29:1, at least 30:1, at least 31:1, at least 32:1, at least 33:1, at least 34:1, at least 35:1, at least 36:1, at least 37:1, at least 38:1, at least 39:1, at least 40:1, at least 41:1, at least 42:1, at least 43:1, at least 44:1, at least 45:1, at least 50:1, at least 55:1, at least 60:1, at least 70:1, at least 75:1, at least 80:1, at least 85:1, at least 90:1, at least 95:1, at least 100:1, at least 110:1, at least 120:1, at least 125:1, at least 130:1, at least 140:1, at least 150:1, at least 160:1, at least 170:1, at least 175:1, at least 180:1, at least 190:1, at least 200:1, or greater.

In some embodiments, a pharmaceutical composition described herein has a molar ratio of 3E10 antibody or variant thereof, or antigen-binding fragment thereof to therapeutic polynucleotide that is 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 50:1, 55:1, 60:1, 70:1, 75:1, 80:1, 85:1, 90:1, 95:1, 100:1, 110:1, 120:1, 125:1, 130:1, 140:1, 150:1, 160:1, 170:1, 175:1, 180:1, 190:1, 200:1, or greater.

In some embodiments, a pharmaceutical composition described herein has a molar ratio of 3E10 antibody or variant thereof, or antigen-binding fragment thereof to therapeutic polynucleotide that is no more than about 200:1. In some embodiments, a pharmaceutical composition described herein has a molar ratio of 3E10 antibody or variant thereof, or antigen-binding fragment thereof to therapeutic polynucleotide that is no more than about 150:1. In some embodiments, a pharmaceutical composition described herein has a molar ratio of 3E10 antibody or variant thereof, or antigen-binding fragment thereof to therapeutic polynucleotide that is no more than about 100:1. In some embodiments, a pharmaceutical composition described herein has a molar ratio of 3E10 antibody or variant thereof, or antigen-binding fragment thereof to therapeutic polynucleotide that is no more than about 50:1. In some embodiments, a pharmaceutical composition described herein has a molar ratio of 3E10 antibody or variant thereof, or antigen-binding fragment thereof to therapeutic polynucleotide that is no more than about 40:1. In some embodiments, a pharmaceutical composition described herein has a molar ratio of 3E10 antibody or variant thereof, or antigen-binding fragment thereof to therapeutic polynucleotide that is no more than about 30:1. In some embodiments, a pharmaceutical composition described herein has a molar ratio of 3E10 antibody or variant thereof, or antigen-binding fragment thereof to therapeutic polynucleotide that is no more than about 25:1. In some embodiments, a pharmaceutical composition described herein has a molar ratio of 3E10 antibody or variant thereof, or antigen-binding fragment thereof to therapeutic polynucleotide that is no more than about 20:1. In some embodiments, a pharmaceutical composition described herein has a molar ratio of 3E10 antibody or variant thereof, or antigen-binding fragment thereof to therapeutic polynucleotide that is no more than about 15:1. In some embodiments, a pharmaceutical composition described herein has a molar ratio of 3E10 antibody or variant thereof, or antigen-binding fragment thereof to therapeutic polynucleotide that is no more than about 10:1.

In some embodiments, a pharmaceutical composition described herein has a molar ratio of 3E10 antibody or variant thereof, or antigen-binding fragment thereof to therapeutic polynucleotide that is no more than about 200:1, no more than about 175:1, no more than about 150:1, no more than about 125:1, no more than about 100:1, no more than about 75:1, no more than about 50:1, no more than about 45:1, no more than about 40:1, no more than about 35:1, no more than about 30:1, no more than about 25:1, no more than about 20:1, or less.

In some embodiments, a pharmaceutical composition described herein has a molar ratio of 3E10 antibody or variant thereof, or antigen-binding fragment thereof to therapeutic polynucleotide that is no more than 200:1. In some embodiments, a pharmaceutical composition described herein has a molar ratio of 3E10 antibody or variant thereof, or antigen-binding fragment thereof to therapeutic polynucleotide that is no more than 150:1. In some embodiments, a pharmaceutical composition described herein has a molar ratio of 3E10 antibody or variant thereof, or antigen-binding fragment thereof to therapeutic polynucleotide that is no more than 100:1. In some embodiments, a pharmaceutical composition described herein has a molar ratio of 3E10 antibody or variant thereof, or antigen-binding fragment thereof to therapeutic polynucleotide that is no more than 50:1. In some embodiments, a pharmaceutical composition described herein has a molar ratio of 3E10 antibody or variant thereof, or antigen-binding fragment thereof to therapeutic polynucleotide that is no more than 40:1. In some embodiments, a pharmaceutical composition described herein has a molar ratio of 3E10 antibody or variant thereof, or antigen-binding fragment thereof to therapeutic polynucleotide that is no more than 30:1. In some embodiments, a pharmaceutical composition described herein has a molar ratio of 3E10 antibody or variant thereof, or antigen-binding fragment thereof to therapeutic polynucleotide that is no more than 25:1. In some embodiments, a pharmaceutical composition described herein has a molar ratio of 3E10 antibody or variant thereof, or antigen-binding fragment thereof to therapeutic polynucleotide that is no more than 20:1. In some embodiments, a pharmaceutical composition described herein has a molar ratio of 3E10 antibody or variant thereof, or antigen-binding fragment thereof to therapeutic polynucleotide that is no more than 15:1. In some embodiments, a pharmaceutical composition described herein has a molar ratio of 3E10 antibody or variant thereof, or antigen-binding fragment thereof to therapeutic polynucleotide that is no more than 10:1.

In some embodiments, a pharmaceutical composition described herein has a molar ratio of 3E10 antibody or variant thereof, or antigen-binding fragment thereof to therapeutic polynucleotide that is no more than 200:1, no more than 175:1, no more than 150:1, no more than 125:1, no more than 100:1, no more than 75:1, no more than 50:1, no more than 45:1, no more than 40:1, no more than 35:1, no more than 30:1, no more than 35:1, no more than 30:1, no more than 25:1, no more than 20:1, or less.

In some embodiments, a pharmaceutical composition described herein has a molar ratio of 3E10 antibody or variant thereof, or antigen-binding fragment thereof to therapeutic polynucleotide that is of from 2:1 to 200:1. In some embodiments, a pharmaceutical composition described herein has a molar ratio of 3E10 antibody or variant thereof, or antigen-binding fragment thereof to therapeutic polynucleotide that is of from 2:1 to 175:1. In some embodiments, a pharmaceutical composition described herein has a molar ratio of 3E10 antibody or variant thereof, or antigen-binding fragment thereof to therapeutic polynucleotide that is of from 2:1 to 150:1. In some embodiments, a pharmaceutical composition described herein has a molar ratio of 3E10 antibody or variant thereof, or antigen-binding fragment thereof to therapeutic polynucleotide that is of from 2:1 to 125:1. In some embodiments, a pharmaceutical composition described herein has a molar ratio of 3E10 antibody or variant thereof, or antigen-binding fragment thereof to therapeutic polynucleotide that is of from 2:1 to 100:1. In some embodiments, a pharmaceutical composition described herein has a molar ratio of 3E10 antibody or variant thereof, or antigen-binding fragment thereof to therapeutic polynucleotide that is of from 2:1 to 75:1. In some embodiments, a pharmaceutical composition described herein has a molar ratio of 3E10 antibody or variant thereof, or antigen-binding fragment thereof to therapeutic polynucleotide that is of from 2:1 to 50:1. In some embodiments, a pharmaceutical composition described herein has a molar ratio of 3E10 antibody or variant thereof, or antigen-binding fragment thereof to therapeutic polynucleotide that is of from 2:1 to 40:1. In some embodiments, a pharmaceutical composition described herein has a molar ratio of 3E10 antibody or variant thereof, or antigen-binding fragment thereof to therapeutic polynucleotide that is of from 2:1 to 30:1. In some embodiments, a pharmaceutical composition described herein has a molar ratio of 3E10 antibody or variant thereof, or antigen-binding fragment thereof to therapeutic polynucleotide that is of from 2:1 to 25:1. In some embodiments, a pharmaceutical composition described herein has a molar ratio of 3E10 antibody or variant thereof, or antigen-binding fragment thereof to therapeutic polynucleotide that is of from 2:1 to 20:1. In some embodiments, a pharmaceutical composition described herein has a molar ratio of 3E10 antibody or variant thereof, or antigen-binding fragment thereof to therapeutic polynucleotide that is of from 2:1 to 15:1. In some embodiments, a pharmaceutical composition described herein has a molar ratio of 3E10 antibody or variant thereof, or antigen-binding fragment thereof to therapeutic polynucleotide that is of from 2:1 to 10:1. In some embodiments, a pharmaceutical composition described herein has a molar ratio of 3E10 antibody or variant thereof, or antigen-binding fragment thereof to therapeutic polynucleotide that is of from 2:1 to 7.5:1. In some embodiments, a pharmaceutical composition described herein has a molar ratio of 3E10 antibody or variant thereof, or antigen-binding fragment thereof to therapeutic polynucleotide that is of from 2:1 to 5:1. In some embodiments, a pharmaceutical composition described herein has a molar ratio of 3E10 antibody or variant thereof, or antigen-binding fragment thereof to therapeutic polynucleotide that is of from 2:1 to 3:1.

In some embodiments, a pharmaceutical composition described herein has a molar ratio of 3E10 antibody or variant thereof, or antigen-binding fragment thereof to therapeutic polynucleotide that is of from 3:1 to 200:1. In some embodiments, a pharmaceutical composition described herein has a molar ratio of 3E10 antibody or variant thereof, or antigen-binding fragment thereof to therapeutic polynucleotide that is of from 3:1 to 175:1. In some embodiments, a pharmaceutical composition described herein has a molar ratio of 3E10 antibody or variant thereof, or antigen-binding fragment thereof to therapeutic polynucleotide that is of from 3:1 to 150:1. In some embodiments, a pharmaceutical composition described herein has a molar ratio of 3E10 antibody or variant thereof, or antigen-binding fragment thereof to therapeutic polynucleotide that is of from 3:1 to 125:1. In some embodiments, a pharmaceutical composition described herein has a molar ratio of 3E10 antibody or variant thereof, or antigen-binding fragment thereof to therapeutic polynucleotide that is of from 3:1 to 100:1. In some embodiments, a pharmaceutical composition described herein has a molar ratio of 3E10 antibody or variant thereof, or antigen-binding fragment thereof to therapeutic polynucleotide that is of from 3:1 to 75:1. In some embodiments, a pharmaceutical composition described herein has a molar ratio of 3E10 antibody or variant thereof, or antigen-binding fragment thereof to therapeutic polynucleotide that is of from 3:1 to 50:1. In some embodiments, a pharmaceutical composition described herein has a molar ratio of 3E10 antibody or variant thereof, or antigen-binding fragment thereof to therapeutic polynucleotide that is of from 3:1 to 40:1. In some embodiments, a pharmaceutical composition described herein has a molar ratio of 3E10 antibody or variant thereof, or antigen-binding fragment thereof to therapeutic polynucleotide that is of from 3:1 to 30:1. In some embodiments, a pharmaceutical composition described herein has a molar ratio of 3E10 antibody or variant thereof, or antigen-binding fragment thereof to therapeutic polynucleotide that is of from 3:1 to 25:1. In some embodiments, a pharmaceutical composition described herein has a molar ratio of 3E10 antibody or variant thereof, or antigen-binding fragment thereof to therapeutic polynucleotide that is of from 3:1 to 20:1. In some embodiments, a pharmaceutical composition described herein has a molar ratio of 3E10 antibody or variant thereof, or antigen-binding fragment thereof to therapeutic polynucleotide that is of from 3:1 to 15:1. In some embodiments, a pharmaceutical composition described herein has a molar ratio of 3E10 antibody or variant thereof, or antigen-binding fragment thereof to therapeutic polynucleotide that is of from 3:1 to 10:1. In some embodiments, a pharmaceutical composition described herein has a molar ratio of 3E10 antibody or variant thereof, or antigen-binding fragment thereof to therapeutic polynucleotide that is of from 3:1 to 7.5:1. In some embodiments, a pharmaceutical composition described herein has a molar ratio of 3E10 antibody or variant thereof, or antigen-binding fragment thereof to therapeutic polynucleotide that is of from 3:1 to 5:1.

In some embodiments, a pharmaceutical composition described herein has a molar ratio of 3E10 antibody or variant thereof, or antigen-binding fragment thereof to therapeutic polynucleotide that is of from 5:1 to 200:1. In some embodiments, a pharmaceutical composition described herein has a molar ratio of 3E10 antibody or variant thereof, or antigen-binding fragment thereof to therapeutic polynucleotide that is of from 5:1 to 175:1. In some embodiments, a pharmaceutical composition described herein has a molar ratio of 3E10 antibody or variant thereof, or antigen-binding fragment thereof to therapeutic polynucleotide that is of from 5:1 to 150:1. In some embodiments, a pharmaceutical composition described herein has a molar ratio of 3E10 antibody or variant thereof, or antigen-binding fragment thereof to therapeutic polynucleotide that is of from 5:1 to 125:1. In some embodiments, a pharmaceutical composition described herein has a molar ratio of 3E10 antibody or variant thereof, or antigen-binding fragment thereof to therapeutic polynucleotide that is of from 5:1 to 100:1. In some embodiments, a pharmaceutical composition described herein has a molar ratio of 3E10 antibody or variant thereof, or antigen-binding fragment thereof to therapeutic polynucleotide that is of from 5:1 to 75:1. In some embodiments, a pharmaceutical composition described herein has a molar ratio of 3E10 antibody or variant thereof, or antigen-binding fragment thereof to therapeutic polynucleotide that is of from 5:1 to 50:1. In some embodiments, a pharmaceutical composition described herein has a molar ratio of 3E10 antibody or variant thereof, or antigen-binding fragment thereof to therapeutic polynucleotide that is of from 5:1 to 40:1. In some embodiments, a pharmaceutical composition described herein has a molar ratio of 3E10 antibody or variant thereof, or antigen-binding fragment thereof to therapeutic polynucleotide that is of from 5:1 to 30:1. In some embodiments, a pharmaceutical composition described herein has a molar ratio of 3E10 antibody or variant thereof, or antigen-binding fragment thereof to therapeutic polynucleotide that is of from 5:1 to 25:1. In some embodiments, a pharmaceutical composition described herein has a molar ratio of 3E10 antibody or variant thereof, or antigen-binding fragment thereof to therapeutic polynucleotide that is of from 5:1 to 20:1. In some embodiments, a pharmaceutical composition described herein has a molar ratio of 3E10 antibody or variant thereof, or antigen-binding fragment thereof to therapeutic polynucleotide that is of from 5:1 to 15:1. In some embodiments, a pharmaceutical composition described herein has a molar ratio of 3E10 antibody or variant thereof, or antigen-binding fragment thereof to therapeutic polynucleotide that is of from 5:1 to 10:1. In some embodiments, a pharmaceutical composition described herein has a molar ratio of 3E10 antibody or variant thereof, or antigen-binding fragment thereof to therapeutic polynucleotide that is of from 5:1 to 7.5:1.

In some embodiments, a pharmaceutical composition described herein has a molar ratio of 3E10 antibody or variant thereof, or antigen-binding fragment thereof to therapeutic polynucleotide that is of from 7.5:1 to 200:1. In some embodiments, a pharmaceutical composition described herein has a molar ratio of 3E10 antibody or variant thereof, or antigen-binding fragment thereof to therapeutic polynucleotide that is of from 7.5:1 to 175:1. In some embodiments, a pharmaceutical composition described herein has a molar ratio of 3E10 antibody or variant thereof, or antigen-binding fragment thereof to therapeutic polynucleotide that is of from 7.5:1 to 150:1. In some embodiments, a pharmaceutical composition described herein has a molar ratio of 3E10 antibody or variant thereof, or antigen-binding fragment thereof to therapeutic polynucleotide that is of from 7.5:1 to 125:1. In some embodiments, a pharmaceutical composition described herein has a molar ratio of 3E10 antibody or variant thereof, or antigen-binding fragment thereof to therapeutic polynucleotide that is of from 7.5:1 to 100:1. In some embodiments, a pharmaceutical composition described herein has a molar ratio of 3E10 antibody or variant thereof, or antigen-binding fragment thereof to therapeutic polynucleotide that is of from 7.5:1 to 75:1. In some embodiments, a pharmaceutical composition described herein has a molar ratio of 3E10 antibody or variant thereof, or antigen-binding fragment thereof to therapeutic polynucleotide that is of from 7.5:1 to 50:1. In some embodiments, a pharmaceutical composition described herein has a molar ratio of 3E10 antibody or variant thereof, or antigen-binding fragment thereof to therapeutic polynucleotide that is of from 7.5:1 to 40:1. In some embodiments, a pharmaceutical composition described herein has a molar ratio of 3E10 antibody or variant thereof, or antigen-binding fragment thereof to therapeutic polynucleotide that is of from 7.5:1 to 30:1. In some embodiments, a pharmaceutical composition described herein has a molar ratio of 3E10 antibody or variant thereof, or antigen-binding fragment thereof to therapeutic polynucleotide that is of from 7.5:1 to 25:1. In some embodiments, a pharmaceutical composition described herein has a molar ratio of 3E10 antibody or variant thereof, or antigen-binding fragment thereof to therapeutic polynucleotide that is of from 7.5:1 to 20:1. In some embodiments, a pharmaceutical composition described herein has a molar ratio of 3E10 antibody or variant thereof, or antigen-binding fragment thereof to therapeutic polynucleotide that is of from 7.5:1 to 15:1. In some embodiments, a pharmaceutical composition described herein has a molar ratio of 3E10 antibody or variant thereof, or antigen-binding fragment thereof to therapeutic polynucleotide that is of from 7.5:1 to 10:1.

In some embodiments, a pharmaceutical composition described herein has a molar ratio of 3E10 antibody or variant thereof, or antigen-binding fragment thereof to therapeutic polynucleotide that is of from 10:1 to 200:1. In some embodiments, a pharmaceutical composition described herein has a molar ratio of 3E10 antibody or variant thereof, or antigen-binding fragment thereof to therapeutic polynucleotide that is of from 10:1 to 175:1. In some embodiments, a pharmaceutical composition described herein has a molar ratio of 3E10 antibody or variant thereof, or antigen-binding fragment thereof to therapeutic polynucleotide that is of from 10:1 to 150:1. In some embodiments, a pharmaceutical composition described herein has a molar ratio of 3E10 antibody or variant thereof, or antigen-binding fragment thereof to therapeutic polynucleotide that is of from 10:1 to 125:1. In some embodiments, a pharmaceutical composition described herein has a molar ratio of 3E10 antibody or variant thereof, or antigen-binding fragment thereof to therapeutic polynucleotide that is of from 10:1 to 100:1. In some embodiments, a pharmaceutical composition described herein has a molar ratio of 3E10 antibody or variant thereof, or antigen-binding fragment thereof to therapeutic polynucleotide that is of from 10:1 to 75:1. In some embodiments, a pharmaceutical composition described herein has a molar ratio of 3E10 antibody or variant thereof, or antigen-binding fragment thereof to therapeutic polynucleotide that is of from 10:1 to 50:1. In some embodiments, a pharmaceutical composition described herein has a molar ratio of 3E10 antibody or variant thereof, or antigen-binding fragment thereof to therapeutic polynucleotide that is of from 10:1 to 40:1. In some embodiments, a pharmaceutical composition described herein has a molar ratio of 3E10 antibody or variant thereof, or antigen-binding fragment thereof to therapeutic polynucleotide that is of from 10:1 to 30:1. In some embodiments, a pharmaceutical composition described herein has a molar ratio of 3E10 antibody or variant thereof, or antigen-binding fragment thereof to therapeutic polynucleotide that is of from 10:1 to 25:1. In some embodiments, a pharmaceutical composition described herein has a molar ratio of 3E10 antibody or variant thereof, or antigen-binding fragment thereof to therapeutic polynucleotide that is of from 10:1 to 20:1. In some embodiments, a pharmaceutical composition described herein has a molar ratio of 3E10 antibody or variant thereof, or antigen-binding fragment thereof to therapeutic polynucleotide that is of from 10:1 to 15:1.

In some embodiments, a pharmaceutical composition described herein has a molar ratio of 3E10 antibody or variant thereof, or antigen-binding fragment thereof to therapeutic polynucleotide that is of from 15:1 to 200:1. In some embodiments, a pharmaceutical composition described herein has a molar ratio of 3E10 antibody or variant thereof, or antigen-binding fragment thereof to therapeutic polynucleotide that is of from 15:1 to 175:1. In some embodiments, a pharmaceutical composition described herein has a molar ratio of 3E10 antibody or variant thereof, or antigen-binding fragment thereof to therapeutic polynucleotide that is of from 15:1 to 150:1. In some embodiments, a pharmaceutical composition described herein has a molar ratio of 3E10 antibody or variant thereof, or antigen-binding fragment thereof to therapeutic polynucleotide that is of from 15:1 to 125:1. In some embodiments, a pharmaceutical composition described herein has a molar ratio of 3E10 antibody or variant thereof, or antigen-binding fragment thereof to therapeutic polynucleotide that is of from 15:1 to 100:1. In some embodiments, a pharmaceutical composition described herein has a molar ratio of 3E10 antibody or variant thereof, or antigen-binding fragment thereof to therapeutic polynucleotide that is of from 15:1 to 75:1. In some embodiments, a pharmaceutical composition described herein has a molar ratio of 3E10 antibody or variant thereof, or antigen-binding fragment thereof to therapeutic polynucleotide that is of from 15:1 to 50:1. In some embodiments, a pharmaceutical composition described herein has a molar ratio of 3E10 antibody or variant thereof, or antigen-binding fragment thereof to therapeutic polynucleotide that is of from 15:1 to 40:1. In some embodiments, a pharmaceutical composition described herein has a molar ratio of 3E10 antibody or variant thereof, or antigen-binding fragment thereof to therapeutic polynucleotide that is of from 15:1 to 30:1. In some embodiments, a pharmaceutical composition described herein has a molar ratio of 3E10 antibody or variant thereof, or antigen-binding fragment thereof to therapeutic polynucleotide that is of from 15:1 to 25:1. In some embodiments, a pharmaceutical composition described herein has a molar ratio of 3E10 antibody or variant thereof, or antigen-binding fragment thereof to therapeutic polynucleotide that is of from 15:1 to 20:1.

In some embodiments, a pharmaceutical composition described herein has a molar ratio of 3E10 antibody or variant thereof, or antigen-binding fragment thereof to therapeutic polynucleotide that is of from 20:1 to 200:1. In some embodiments, a pharmaceutical composition described herein has a molar ratio of 3E10 antibody or variant thereof, or antigen-binding fragment thereof to therapeutic polynucleotide that is of from 20:1 to 175:1. In some embodiments, a pharmaceutical composition described herein has a molar ratio of 3E10 antibody or variant thereof, or antigen-binding fragment thereof to therapeutic polynucleotide that is of from 20:1 to 150:1. In some embodiments, a pharmaceutical composition described herein has a molar ratio of 3E10 antibody or variant thereof, or antigen-binding fragment thereof to therapeutic polynucleotide that is of from 20:1 to 125:1. In some embodiments, a pharmaceutical composition described herein has a molar ratio of 3E10 antibody or variant thereof, or antigen-binding fragment thereof to therapeutic polynucleotide that is of from 20:1 to 100:1. In some embodiments, a pharmaceutical composition described herein has a molar ratio of 3E10 antibody or variant thereof, or antigen-binding fragment thereof to therapeutic polynucleotide that is of from 20:1 to 75:1. In some embodiments, a pharmaceutical composition described herein has a molar ratio of 3E10 antibody or variant thereof, or antigen-binding fragment thereof to therapeutic polynucleotide that is of from 20:1 to 50:1. In some embodiments, a pharmaceutical composition described herein has a molar ratio of 3E10 antibody or variant thereof, or antigen-binding fragment thereof to therapeutic polynucleotide that is of from 20:1 to 40:1. In some embodiments, a pharmaceutical composition described herein has a molar ratio of 3E10 antibody or variant thereof, or antigen-binding fragment thereof to therapeutic polynucleotide that is of from 20:1 to 30:1. In some embodiments, a pharmaceutical composition described herein has a molar ratio of 3E10 antibody or variant thereof, or antigen-binding fragment thereof to therapeutic polynucleotide that is of from 20:1 to 25:1.

In some embodiments, a pharmaceutical composition described herein has a molar ratio of 3E10 antibody or variant thereof, or antigen-binding fragment thereof to therapeutic polynucleotide that is of from 25:1 to 200:1. In some embodiments, a pharmaceutical composition described herein has a molar ratio of 3E10 antibody or variant thereof, or antigen-binding fragment thereof to therapeutic polynucleotide that is of from 25:1 to 175:1. In some embodiments, a pharmaceutical composition described herein has a molar ratio of 3E10 antibody or variant thereof, or antigen-binding fragment thereof to therapeutic polynucleotide that is of from 25:1 to 150:1. In some embodiments, a pharmaceutical composition described herein has a molar ratio of 3E10 antibody or variant thereof, or antigen-binding fragment thereof to therapeutic polynucleotide that is of from 25:1 to 125:1. In some embodiments, a pharmaceutical composition described herein has a molar ratio of 3E10 antibody or variant thereof, or antigen-binding fragment thereof to therapeutic polynucleotide that is of from 25:1 to 100:1. In some embodiments, a pharmaceutical composition described herein has a molar ratio of 3E10 antibody or variant thereof, or antigen-binding fragment thereof to therapeutic polynucleotide that is of from 25:1 to 75:1. In some embodiments, a pharmaceutical composition described herein has a molar ratio of 3E10 antibody or variant thereof, or antigen-binding fragment thereof to therapeutic polynucleotide that is of from 25:1 to 50:1. In some embodiments, a pharmaceutical composition described herein has a molar ratio of 3E10 antibody or variant thereof, or antigen-binding fragment thereof to therapeutic polynucleotide that is of from 25:1 to 40:1. In some embodiments, a pharmaceutical composition described herein has a molar ratio of 3E10 antibody or variant thereof, or antigen-binding fragment thereof to therapeutic polynucleotide that is of from 25:1 to 30:1.

In yet other embodiments, other ranges falling with the range of about 2:1 to about 200:1 are contemplated.

In some embodiments, a pharmaceutical composition described herein has a molar ratio of 3E10 antibody or variant thereof, or antigen-binding fragment thereof to therapeutic polynucleotide that is of from about 1:1 to about 200:1. In some embodiments, a pharmaceutical composition described herein has a molar ratio of 3E10 antibody or variant thereof, or antigen-binding fragment thereof to therapeutic polynucleotide that is of from about 1:1 to about 175:1. In some embodiments, a pharmaceutical composition described herein has a molar ratio of 3E10 antibody or variant thereof, or antigen-binding fragment thereof to therapeutic polynucleotide that is of from about 1:1 to about 150:1. In some embodiments, a pharmaceutical composition described herein has a molar ratio of 3E10 antibody or variant thereof, or antigen-binding fragment thereof to therapeutic polynucleotide that is of from about 1:1 to about 125:1. In some embodiments, a pharmaceutical composition described herein has a molar ratio of 3E10 antibody or variant thereof, or antigen-binding fragment thereof to therapeutic polynucleotide that is of from about 1:1 to about 100:1. In some embodiments, a pharmaceutical composition described herein has a molar ratio of 3E10 antibody or variant thereof, or antigen-binding fragment thereof to therapeutic polynucleotide that is of from about 1:1 to about 75:1. In some embodiments, a pharmaceutical composition described herein has a molar ratio of 3E10 antibody or variant thereof, or antigen-binding fragment thereof to therapeutic polynucleotide that is of from about 1:1 to about 50:1. In some embodiments, a pharmaceutical composition described herein has a molar ratio of 3E10 antibody or variant thereof, or antigen-binding fragment thereof to therapeutic polynucleotide that is of from about 1:1 to about 30:1. In some embodiments, a pharmaceutical composition described herein has a molar ratio of 3E10 antibody or variant thereof, or antigen-binding fragment thereof to therapeutic polynucleotide that is of from about 1:1 to about 20:1. In some embodiments, a pharmaceutical composition described herein has a molar ratio of 3E10 antibody or variant thereof, or antigen-binding fragment thereof to therapeutic polynucleotide that is of from about 1:1 to about 10:1. In some embodiments, a pharmaceutical composition described herein has a molar ratio of 3E10 antibody or variant thereof, or antigen-binding fragment thereof to therapeutic polynucleotide that is of from about 1:1 to about 5:1.

As reported in Example 3, 3E10 antibodies or variants thereof, or antigen-binding fragments thereof localize to skeletal muscle tissue in vivo following systemic administration. Accordingly, the compositions described herein are well suited for the delivery of therapeutic mRNAs encoding proteins useful for treating disorders of skeletal muscle tissue. Accordingly, in some embodiments, the therapeutic mRNA polynucleotide encodes a skeletal muscle polypeptide.

Examples of proteins, and their associated genes, that are mutated in various myopathies are presented in Table 2. Generally, any one of these proteins, and variants thereof retaining a function of the full-length protein, can be encoded by the therapeutic mRNAs disclosed herein. Accordingly, in some embodiments, the skeletal-muscle polypeptide is selected from the group consisting of nebulin (NEB), skeletal muscle alpha-actin (ACTA), alpha-tropomyosin-3 (TPM3), beta-tropomyosin-2 (TPM2), troponin T1 (TNNT1), cofilin-2 (CFL2), Kelch-repeat-and-BTB-domain-containing-13 (KBTBD13), Kelch-like-family member-40 (KLHL40), Kelch-like protein 4 (KLHL4), Kelch-like-family member 41 (KLHL41), leiomodin-3 (LMOD3), myopalladin (MYPN), ryanodine receptor (RYR1), selenoprotein N (SEPN1), myotubularin (MTM1), dynamin-2 (DNM2), amphiphysin-2 (BIN1), titin (TTN), striated muscle preferentially expressed protein kinase (SPEG), slow-skeletal/beta-cardiac myosin heavy chain (MYH7) cytochrome b, cytochrome c oxidase, thymidine kinase (TK2), polymerase gamma 1 (POLG1), lysosomal enzyme acid alpha-glucosidase (GAA), glycogen-debranching enzyme (AGL), myophosphorylase (PYGM), carnitine transporter OCTN2 (SLC22A5), electron-transfer flavoprotein (ETF), ETF-dehydrogenase (ETFH), adipose triglyceride lipase (PNPLA2), skeletal muscle chloride channel (CIC1), alpha-subunit of the skeletal muscle sodium channel (SCN4A), myotonin-protein kinase (DMPK), zinc finger 9 (ZNF9), dystrophin (DMD), myotilin (MYOT), lamin A/C (LMNA), caveolin 3 (CAV3), DnaJ Heat Shock Protein Family (Hsp40) Member B6 (DNAJB6), desmin (DES), transportin 3, Heterogeneous nuclear ribonucleoprotein D-like (HNRPDL), calpain 3, dysferlin (DYSF), gamma-sarcoglycan (SGCG), alpha-sarcoglycan (SGCA), beta-sarcoglycan (SGCB), delta-sarcoglycan (SGCD), telethonin (TCAP), E3 ubiquitin-protein ligase TRIM32 (TRIM32), Fukutin-related protein (FKRP), Protein O-mannosyl-transferase 1 (POMT1), anoctamin 5 (ANO5), fukutin, Protein O-mannosyl-transferase 2 (POMT2), O-linked-mannose beta-1,2-N acetylglucosaminyltransferase (POMTnG1), dystroglycan (DAG1), plectin (PLEC1), LGMD2R, Trafficking protein particle complex subunit 11 (TRAPPC11), Mannose-1-phosphate guanyltransferase beta (GMPPB), D-ribitol-5-phosphate cytidylyltransferase (ISPD), alpha-glucosidase, LIM and senescent cell antigen-like-containing domain protein 2 (LIMS2), isoprenoid synthase domain containing (ISPD), Popeye-domain containing 1 (POPDC1), lamina-associated polypeptide 1B (TORIAIP1), Oglucosyltransferase 1 (POGLUT1), Laminin subunit alpha-2 (LAMA2), collagen alpha-1(VI) chain (COL6A1), collagen alpha-2(VI) chain (COL6A2), collagen alpha-3(VI) chain (COL6A3), double homeobox 4 (DUX4), and emerin (EMD).

Moreover, because 3E10 antibodies or variants thereof, or antigen-binding fragments thereof localize to skeletal muscle tissue in vivo following systemic administration, the compositions of the present disclosure can be formulated for, and subsequently administered by, one of many common administrative routes. In some embodiments, the pharmaceutical composition is formulated for parenteral administration. In some embodiments, the parenteral administration is intramuscular administration, intravenous administration, or subcutaneous administration.

In some embodiments, the mRNA of the compositions described herein are codon-optimized, e.g., to improve half-life or increase translation in skeletal muscle tissue. Codon-optimized refers to a polynucleotide sequence encoding a polypeptide (e.g., a skeletal-muscle polypeptide), where at least one codon of the native polynucleotide encoding the polypeptide has been changed to improve a property of the polynucleotide sequence. In some embodiments, the improved property promotes increased transcription of mRNA coding for the polypeptide, increased stability of the mRNA (e.g., improved mRNA half-life), increased translation of the polypeptide, and/or increased packaging of the polynucleotide within the vector. Non-limiting examples of alterations that can be used to achieve the improved properties include changing the usage and/or distribution of codons for particular amino acids, adjusting global and/or local GC content, removing AT-rich sequences, removing repeated sequence elements, adjusting global and/or local CpG dinucleotide content, removing cryptic regulatory elements (e.g., TATA box and CCAAT box elements), removing of intron/exon splice sites, improving regulatory sequences (e.g., introduction of a Kozak consensus sequence), and removing sequence elements capable of forming secondary structure (e.g., stem-loops) in the transcribed mRNA.

Similarly, in some embodiments, the mRNA of the compositions described herein include one or more non-canonical nucleotides, e.g., to improve the stability and/or half-life of the mRNA in vivo. Examples of non-canonical nucleotides suitable for inclusion in the mRNA molecules described herein are described in U.S. Pat. No. 9,181,319, the content of which is incorporated herein by reference.

### EXAMPLES

With respect to the experiments below, standard 3E10 sequence was used except wherein noted to be the D31N variant (e.g., Example 4). Both standard 3E10 and the D31N variant were used as full length antibodies.

### Example 1—Carrier DNA Enhances mRNA to Non-Tumor Tissue

2 ug of fluorescently labeled mRNA was mixed with 20 ug of 3E10-D31N with or without carrier DNA (5 ug) for 15 minutes at room temperature. mRNA complexed to 3E10 was injected to fetuses at E15.5. 24-48 hours after treatment, fetuses were harvested and analyzed for mRNA delivery using IVIS imaging.

Without carrier DNA, 3E10-D31N complexed to mRNA was rapidly cleared from fetuses at 24 hours. The addition of carrier DNA, however, resulted in detectable mRNA signal in multiple tissues of the fetus at 48 hours.

### Example 2-3E10 (D31N) Complexed with mRNA and Carrier DNA Results in Sustained Levels Protein Expression

10 ug of luciferase mRNA and 10 ug of single stranded carrier DNA (60 nts) was mixed with 100 ug of 3E10 (WT) or 3E10 (D31N) for 15 minutes at room temperature. mRNA complexed to 3E10 was injected intramuscularly (IM) in the right quadricep of each mouse. Luciferase expression was monitored over 6 days.

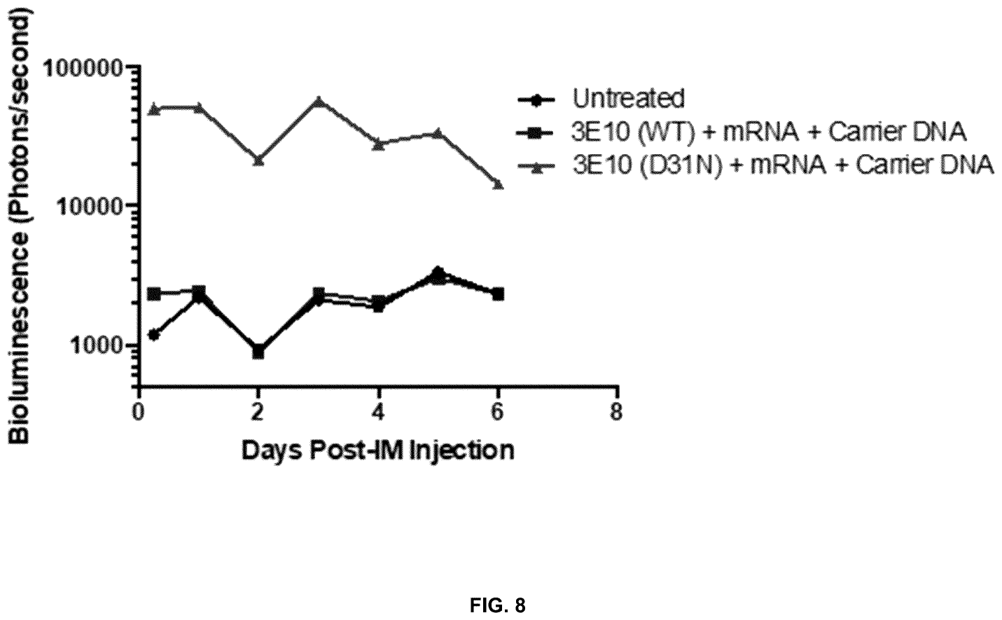

As seen in FIG. 8, administration of 3E10 (D31N) complexed with mRNA and carrier DNA resulted in sustained levels of luciferase expression, while 3E10 (WT) complexed to mRNA and carrier DNA failed to produce any appreciable signal above background.

### Example 3—Distribution of IV Injected 3E10 in Vivo

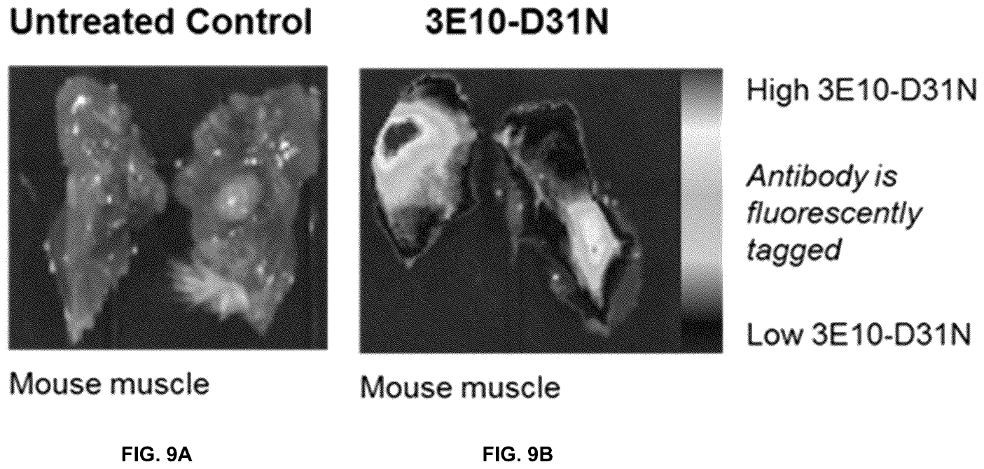

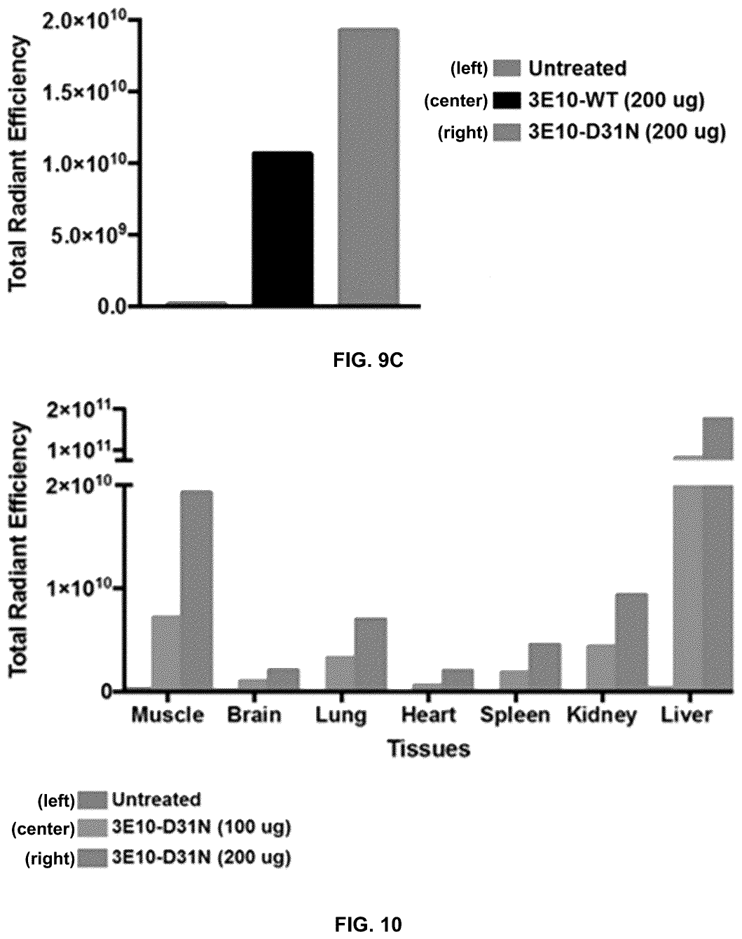

Distribution of IV injected 3E10 to muscle was investigated. Mice were injected intravenously with 200 μg of 3E10, WT or D31N, labeled with VivoTag680 (Perkin Elmer). Four hours after injection, muscle was harvested and imaged by IVIS (Perkin Elmer) (FIGS. 9A and 9B). Quantification of IVIS image demonstrates that 3E10-D31N achieves higher distribution to muscle when compared to 3E10-WT (FIG. 9C).

Dose-dependent biodistribution of 3E10-D31N to tissues was investigated. Mice were injected intravenously with 100 μg or 200 μg of 3E10-D31N labeled with VivoTag680 (Perkin Elmer). 24 hours after injection, tissues were harvested and imaged by IVIS (Perkin Elmer). Quantification of tissue distribution demonstrated a dose-dependent, two-fold increase in muscle accumulation without a commensurate increase in multiple tissues including liver (FIG. 10).

### Example 4—Molecular Modeling of 3E10 and Engineered Variants Thereof

WT HEAVY CHAIN scFv SEQUENCE
(SEQ ID NO: 108)
E VQLVESGGGL VKPGGSRKLS CAASGFTFSD YGMHWVRQAP
EKGLEWVAYI SSGSSTIYYA DTVKGRFTIS RDNAKNTLFL
QMTSLRSEDT AMYYCARRGL LLDYWGQGTT LTVS
LIGHT CHAIN scFv SEQUENCE
(SEQ ID NO: 109)
D IVLTQSPASL AVSLGQRATI SCRASKSVST SSYSYMHWYQ
QKPGQPPKLL IKYASYLESG VPARFSGSGS GTDFTLNIHP
VEEEDAATYY CQHSREFPWT FGGGTKLEIK RADAAPGGGG
SGGGGSGGGGS

Molecular modeling of 3E10 (Pymol) revealed a putative Nucleic Acid Binding pocket (NAB1) (FIGS. 11A-11B). Mutation of aspartic acid at residue 31 of CDR1 to asparagine increased the cationic charge of this residue and enhanced nucleic acid binding and delivery in vivo (3E10-D31N).

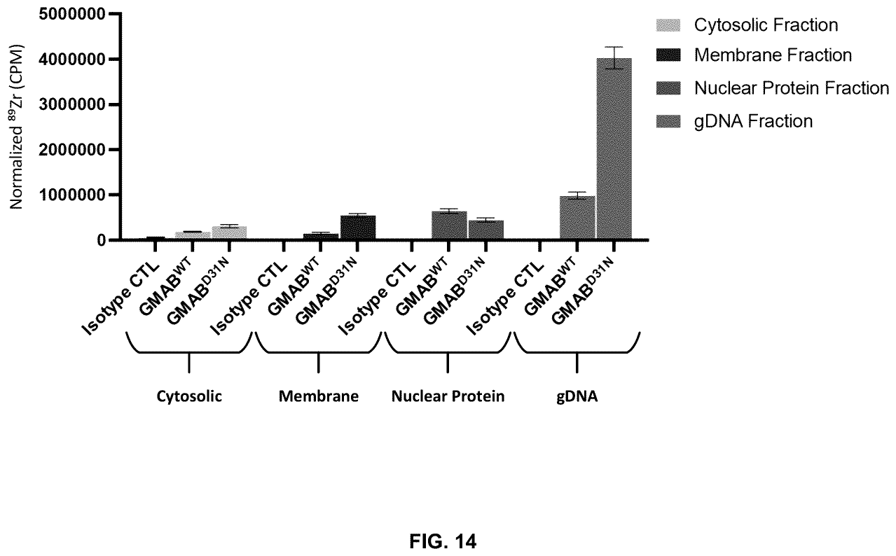

Mutation of aspartic acid at residue 31 of CDR1 to arginine (3E10-D31R), further expanded the cationic charge while mutation to lysine (3E10-D31K) changed charge orientation (FIG. 14A).

NAB1 amino acids predicted from molecular modeling have been underlined in the heavy and light chain sequences above. FIG. 11B is an illustration showing molecular modeling of 3E10-scFv (Pymol) with NAB1 amino acid residues illustrated with punctate dots.

### Example 5—Intermuscular Injection of 3E10 (D31N) Complexed with mRNA Results in Sustained Protein Expression in Skeletal Muscle

It was next investigated whether intramuscular administration of a 3E10 (D31N)-mRNA complex would result in sustained expression of the mRNA in skeletal muscle. Briefly, complexes of 3E10 (D31N) and mRNA encoding green fluorescent protein, a luciferase, having the sequence GFP_mRNA shown below as (SEQ ID NO:110), were formed by mixing 3E10

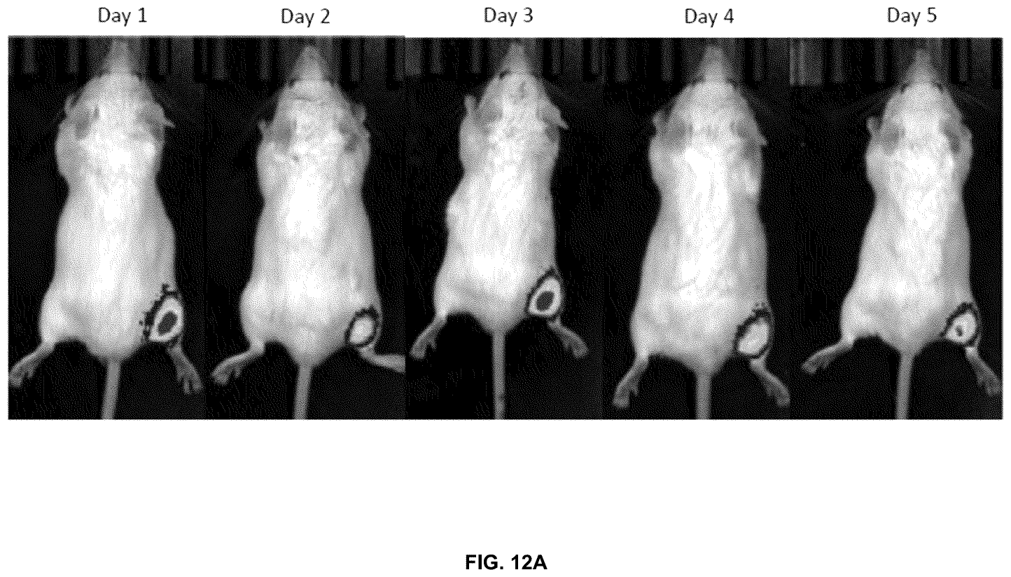

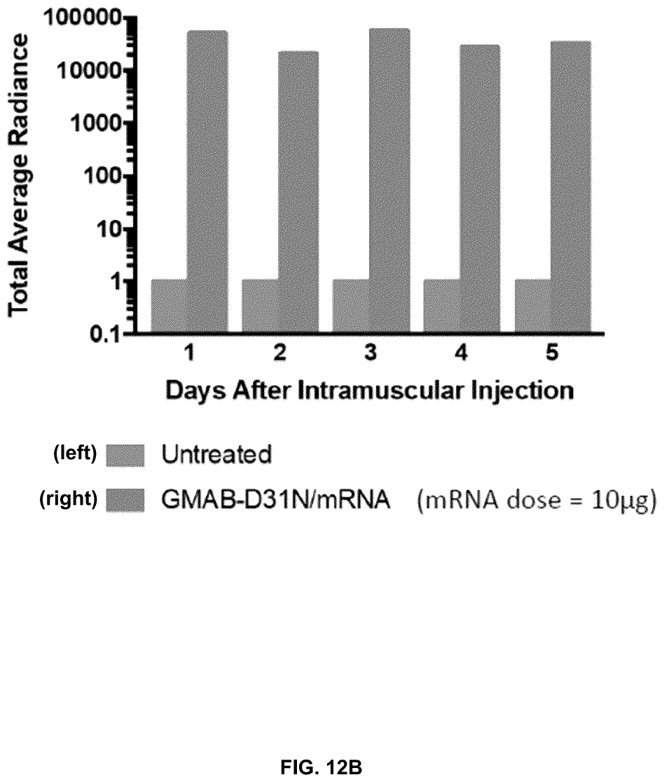

(D31N) and mRNA at a 20:1 molar ratio. The resulting complex was administered by intermuscular injection into the hind-leg skeletal muscle of a mouse. Bioluminescence in the skeletal muscle, indicating expression of the luciferase from the injected mRNA, was imaged (FIG. 12A) and quantified (FIG. 12B) over five days. As shown in FIG. 12B, expression levels of the mRNA-encoded luciferase were sustained for at least five days.

### Example 6-3E10 (D31N) Protects mRNA Against RNA Degradation

It was next investigated whether complexing mRNA with 3E10 (D31N) would protect the mRNA from degradation. Briefly, complexes of 3E10 (D31N) and mRNA encoding green fluorescent protein, a luciferase, having the sequence GFP_mRNA shown below as (SEQ ID NO:110), were formed by mixing 3E10 (D31N) and mRNA at a 20:1 molar ratio. The free mRNA and the 3E10-mRNA complex were then incubated with 1% serum, 10% serum, or 16 μg/mL RNAse A for 10 minutes at 37° C. Gel electrophoresis analysis of the reactions was performed (FIG. 13A). As shown in FIG. 13A, free mRNA was degraded by incubation with each of 1% serum, 10% serum, and RNAse A. However, no apparent RNA degradation was observed when the complexed mRNA was incubated with any of 1% serum, 10% serum, or RNAse A, suggesting that 3E10 (D31N) protects mRNA from degradation.

GFP_mRNA-
(SEQ ID NO: 110)
AUGGUGAGCAAGGGCGAGGAGCUGUUCACCGGGGUGGUGCCCAUCCUGG
UCGAGCUGGACGGCGACGUAAACGGCCACAAGUUCAGCGUGUCCGGCGA
GGGCGAGGGCGAUGCCACCUACGGCAAGCUGACCCUGAAGUUCAUCUGC
ACCACCGGCAAGCUGCCCGUGCCCUGGCCCACCCUCGUGACCACCCUGA
CCUACGGCGUGCAGUGCUUCAGCCGCUACCCCGACCACAUGAAGCAGCA
CGACUUCUUCAAGUCCGCCAUGCCCGAAGGCUACGUCCAGGAGCGCACC
AUCUUCUUCAAGGACGACGGCAACUACAAGACCCGCGCCGAGGUGAAGU
UCGAGGGCGACACCCUGGUGAACCGCAUCGAGCUGAAGGGCAUCGACUU
CAAGGAGGACGGCAACAUCCUGGGGCACAAGCUGGAGUACAACUACAAC
AGCCACAACGUCUAUAUCAUGGCCGACAAGCAGAAGAACGGCAUCAAGG
UGAACUUCAAGAUCCGCCACAACAUCGAGGACGGCAGCGUGCAGCUCGC
CGACCACUACCAGCAGAACACCCCCAUCGGCGACGGCCCCGUGCUGCUG
CCCGACAACCACUACCUGAGCACCCAGUCCGCCCUGAGCAAAGACCCCA
ACGAGAAGCGCGAUCACAUGGUCCUGCUGGAGUUCGUGACCGCCGCCGG
GAUCACUCUCGGCAUGGACGAGCUGUACAAGUAA.

Next, it was investigated whether mRNA complexed at lower molar ratios were also protected against RNA degradation. Briefly, complexes of 3E10 (D31N) and mRNA encoding green fluorescent protein (GFP_mRNA; SEQ ID NO:110) were formed by mixing 3E10 (D31N) and mRNA at a 2:1 molar ratio. The free mRNA and the 3E10-mRNA complex were then incubated with RNAse A under the conditions described above. Gel electrophoresis analysis of the reactions was performed (FIG. 13B). As shown in FIG. 13B, free mRNA was completely degraded by incubation with RNAse A. However, complexing the mRNA with 3E10 (D31N) at a 2:1 molar ratio resulted in some protection of the mRNA against degradation, as indicated by the presence of an RNA signal in the well, indicating the presence of intact 3E10 (D31N)-mRNA complex. The protection provided at a 2:1 molar ratio appears to be less than the protection afforded the mRNA when complexed at a 20:1 molar ratio.

### Example 7-3E10-D31N is Internalized and Associates with gDNA In Vivo

Internalization and cellular location experiments for 3E10-D31N were investigated. Isotype control, 3E10-WT (GMAB^{WT}), and 3E10-D31N (GMAB^{D31N}) antibodies were labeled with ⁸⁹Zr and administered to cells in vivo. After an amount of time, cellular components (cytosol, membrane, nuclear protein, and gDNA) were fractionated and assayed for ⁸⁹Zr signal (Counts per Minute, CPM). As shown in FIG. 14, the majority of the internalized GMAB^{WT} and GMAB^{D31N} antibodies localized to the nucleus and were found associated with both the nuclear protein fraction of the nucleus and the gDNA fraction. However, a larger portion of GMAB^{D31N} associated with the gDNA fraction than did GMABWT, suggesting that GMABD31N localizes more readily to chromatin than does GMAB^{WT}

### Example 8: 3E10 (D31N) Protects Dystrophin mRNA Against RNA Degradation

It was investigated whether 3E10-D31N would protect mRNA encoding dystrophin from enzymatic degradation when complexed, and whether larger stochiometric amounts of 3E10-D31N were necessary. Briefly, complexes of 3E10-D31N and a 14 kb mRNA encoding full-length human dystrophin, were formed by mixing 3E10-D31N and mRNA at 1:1, 2:1, 5:1, 10:1, 20:1, and 100:1 molar ratios (3E10:mRNA). The free mRNA and the 3E10-mRNA complexes were then incubated with 6 μg/mL RNAse A for 10 minutes at 37° C. with the addition proteinase K to facilitate protein degradation. FIG. 15 shows agarose gel electrophoresis analysis of the protection assays. As shown in FIG. 15, free dystrophin mRNA, as well as dystrophin mRNA complexed at 1:1, 2:1, 5:1, and 10:1 molar ratios (3E10:mRNA) was completely degraded by incubation with RNAse A. However, as shown in FIG. 15, complexing the dystrophin mRNA with 3E10 at a molar ratio of 20:1 and 100:1 afforded increasing protection of the mRNA from degradation by RNAse A, as indicated by bands migrating at a similar distance as undegraded mRNA on the gel. These results, coupled with those of Example 6, suggest that 3E10 protects polynucleotides in a size-dependent manner.

### REFERENCES CITED AND ALTERNATIVE EMBODIMENTS

All references cited herein are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only. The embodiments were chosen and described in order to best explain the principles of the invention and its practical applications, to thereby enable others skilled in the art to best utilize the invention and various embodiments with various modifications as are suited to the particular use contemplated. The invention is to be limited only by the terms of the appended claims, along with the full scope of equivalents to which such claims are entitled.

What is claimed is:

1. A method for treating a genetic skeletal muscle disease in a subject in need thereof, the method comprising:
parenterally administering a therapeutically effective amount of a composition comprising a complex formed between (i) a 3E10 antibody or antigen-binding fragment thereof, and (ii) a therapeutic mRNA polynucleotide encoding a skeletal muscle protein;
wherein the 3E10 antibody or antigen-binding fragment thereof comprises:
(a) a light chain variable region (VL) complementarity determining region (CDR) 1 comprising the amino acid sequence of 3E10-VL-CDRm (SEQ ID NO:60),
(b) a VL CDR2 comprising the amino acid sequence of 3E10-VL-CDR2m (SEQ ID NO:62),
(c) a VL CDR3 comprising the amino acid sequence of 3E10-VL-CDR3m (SEQ ID NO:63),
(d) a heavy chain variable region (VH) CDR1 comprising the amino acid sequence of 3E10-VH-CDR1m (SEQ ID NO:58),
(e) a VH CDR2 comprising the amino acid sequence of 3E10-VH-CDR2m (SEQ ID NO:59), and
(f) a VH CDR3 comprising the amino acid sequence of 3E10-VH-CDR3m (SEQ ID NO:60).

2. The method of claim 1, wherein the therapeutic mRNA polynucleotide encodes a skeletal muscle polypeptide for which the subject has a loss-of-function mutation.

3. The method of claim 1, wherein the 3E10 antibody or antigen-binding fragment thereof comprises:
(a) a light chain variable region (VL) complementarity determining region (CDR) 1 comprising the amino acid sequence of 3E10-VL-CDR1 (SEQ ID NO:9),
(b) a VL CDR2 comprising the amino acid sequence of 3E10-VL-CDR2 (SEQ ID NO:10),
(c) a VL CDR3 comprising the amino acid sequence of 3E10-VL-CDR3 (SEQ ID NO:11),
(d) a heavy chain variable region (VH) CDR1 comprising the amino acid sequence of 3E10-VH-CDR1a (SEQ ID NO:16),
(e) a VH CDR2 comprising the amino acid sequence of 3E10-VH-CDR2 (SEQ ID NO:4), and
(f) a VH CDR3 comprising the amino acid sequence of 3E10-VH-CDR3 (SEQ ID NO:5).

4. The method of claim 1, wherein the genetic skeletal muscle disease is a non-dystrophic genetic myopathy.

5. The method of claim 1, wherein the genetic skeletal muscle disease is a dystrophic genetic myopathy.

6. The method of claim 1, wherein the parenteral administration is intramuscular administration, intravenous administration, or subcutaneous administration.

7. The method of claim 1, wherein the composition comprises a molar ratio of (i) 3E10 antibody or variant thereof, or antigen-binding fragment thereof to (ii) therapeutic polynucleotide of at least 2:1.

8. The method of claim 7, wherein the composition comprises a molar ratio of (i) 3E10 antibody or antigen-binding fragment thereof to (ii) therapeutic polynucleotide of at least 5:1.

9. The method of claim 7, wherein the composition comprises a molar ratio of (i) 3E10 antibody or antigen-binding fragment thereof to (ii) therapeutic polynucleotide of at least 20:1.

10. The method of claim 7, wherein the composition comprises a molar ratio of (i) 3E10 antibody or antigen-binding fragment thereof to (ii) therapeutic polynucleotide of at least 50:1.

11-15. (canceled)

16. The method of claim 1, wherein the composition comprises a molar ratio of (i) 3E10 antibody or antigen-binding fragment thereof to (ii) therapeutic polynucleotide of from 2:1 to 50:1, wherein the therapeutic polynucleotide is no more than 2000 nucleotides in length.

17. The method of claim 1, wherein the composition comprises a molar ratio of (i) 3E10 antibody or antigen-binding fragment thereof to (ii) therapeutic polynucleotide of from 2:1 to 30:1, wherein the therapeutic polynucleotide is no more than 1000 nucleotides in length.

18. (canceled)

19. The method of claim 1, wherein the subject carries at least one variant form of a gene encoding the skeletal-muscle protein.

20. The method of claim 1, wherein the skeletal-muscle polypeptide is selected from the group consisting of nebulin (NEB), skeletal muscle alpha-actin (ACTA), alpha-tropomyosin-3 (TPM3), beta-tropomyosin-2 (TPM2), troponin T1 (TNNT1), cofilin-2 (CFL2), Kelch-repeat-and-BTB-domain-containing-13 (KBTBD13), Kelch-like-family member-40 (KLHL40), Kelch-like protein 4 (KLHL4), Kelch-like-family member 41 (KLHL41), leiomodin-3 (LMOD3), myopalladin (MYPN), ryanodine receptor (RYR1), selenoprotein N (SEPN1), myotubularin (MTM1), dynamin-2 (DNM2), amphiphysin-2 (BIN1), titin (TTN), striated muscle preferentially expressed protein kinase (SPEG), slow-skeletal/beta-cardiac myosin heavy chain (MYH7) cytochrome b, cytochrome c oxidase, thymidine kinase (TK2), polymerase gamma 1 (POLG1), lysosomal enzyme acid alpha-glucosidase (GAA), glycogen-debranching enzyme (AGL), myophosphorylase (PYGM), carnitine transporter OCTN2 (SLC22A5), electron-transfer flavoprotein (ETF), ETF-dehydrogenase (ETFH), adipose triglyceride lipase (PNPLA2), skeletal muscle chloride channel (CIC1), alpha-subunit of the skeletal muscle sodium channel (SCN4A), myotonin-protein kinase (DMPK), zinc finger 9 (ZNF9), dystrophin (DMD), myotilin (MYOT), lamin A/C (LMNA), caveolin 3 (CAV3), DnaJ Heat Shock Protein Family (Hsp40) Member B6 (DNAJB6), desmin (DES), transportin 3, Heterogeneous nuclear ribonucleoprotein D-like (HNRPDL), calpain 3, dysferlin (DYSF), gamma-sarcoglycan (SGCG), alpha-sarcoglycan (SGCA), beta-sarcoglycan (SGCB), delta-sarcoglycan (SGCD), telethonin (TCAP), E3 ubiquitin-protein ligase TRIM32 (TRIM32), Fukutin-related protein (FKRP), Protein O-mannosyl-transferase 1 (POMT1), anoctamin 5 (ANO5), fukutin, Protein O-mannosyl-transferase 2 (POMT2), O-linked-mannose beta-1,2-N acetylglucosaminyltransferase (POMTnG1), dystroglycan (DAG1), plectin (PLEC1), LGMD2R, Trafficking protein particle complex subunit 11 (TRAPPC11), Mannose-1-phosphate guanyltransferase beta (GMPPB), D-ribitol-5-phosphate cytidylyltransferase (ISPD), alpha-glucosidase, LIM and senescent cell antigen-like-containing domain protein 2 (LIMS2), isoprenoid synthase domain containing (ISPD), Popeye-domain containing 1 (POPDC1), lamina-associated polypeptide 1B (TOR1AIP1), Oglucosyltransferase 1 (POGLUT1), Laminin subunit alpha-2 (LAMA2), collagen alpha-1(VI) chain (COL6A1), collagen alpha-2(VI) chain (COL6A2), collagen alpha-3(VI) chain (COL6A3), double homeobox 4 (DUX4), and emerin (EMD).

21. The method of claim 1, wherein the 3E10 antibody or antigen-binding fragment thereof comprises a VL comprising an amino acid sequence that is at least 85% identical to 3E10-VL (SEQ ID NO:8).

22. (canceled)

23. The method of claim 1, wherein the 3E10 antibody or antigen-binding fragment thereof comprises a VH comprising an amino acid sequence that is at least 85% identical to 3E10-VH (SEQ ID NO:2).

24. (canceled)

25. The method of claim 1, wherein the 3E10 antibody or antigen-binding fragment thereof comprises:
a heavy chain comprising, from N- to C-terminal, VH-CH1-hinge-CH2-CH3, and a light chain comprising, from N- to C-terminal, VL-CL.

26. The method of claim 25, wherein the hinge-CH2-CH3 is an Fc domain selected from the group consisting of the Fc domain from human IgG1, IgG2, IgG3 and IgG4.

27.-61. (canceled)

62. A pharmaceutical composition comprising a complex formed between (i) a 3E10 antibody or antigen-binding fragment thereof, and (ii) a therapeutic mRNA polynucleotide, wherein the pharmaceutical composition comprises a molar ratio of (a) 3E10 antibody or antigen-binding fragment thereof to (b) therapeutic polynucleotide of at least 2:1; and
wherein the 3E10 antibody or antigen-binding fragment thereof comprises:
(a) a light chain variable region (VL) complementarity determining region (CDR) 1 comprising the amino acid sequence of 3E10-VL-CDRm (SEQ ID NO:60),
(b) a VL CDR2 comprising the amino acid sequence of 3E10-VL-CDR2m (SEQ ID NO:62),
(c) a VL CDR3 comprising the amino acid sequence of 3E10-VL-CDR3m (SEQ ID NO:63),
(d) a heavy chain variable region (VH) CDR1 comprising the amino acid sequence of 3E10-VH-CDR1m (SEQ ID NO:58),
(e) a VH CDR2 comprising the amino acid sequence of 3E10-VH-CDR2m (SEQ ID NO:59), and
(f) a VH CDR3 comprising the amino acid sequence of 3E10-VH-CDR3m (SEQ ID NO:60).

63.-84. (canceled)

85. A pharmaceutical composition comprising a complex formed between (i) a 3E10 antibody or antigen-binding fragment thereof that binds to nucleic acids, and (ii) a therapeutic mRNA polynucleotide, wherein:
the antibody or antigen-binding fragment thereof comprises:
(a) a light chain variable region (VL) complementarity determining region (CDR) 1 comprising an amino acid sequence having no more than two amino acid substitutions relative to 3E10-VL-CDR1 (SEQ ID NO:9),
(b) a VL CDR2 comprising an amino acid sequence having no more than two amino acid substitutions relative to 3E10-VL-CDR2 (SEQ ID NO:10),
(c) a VL CDR3 comprising an amino acid sequence having no more than two amino acid substitutions relative to 3E10-VL-CDR3 (SEQ ID NO:11),
(d) a heavy chain variable region (VH) CDR1 comprising an amino acid sequence having no more than two amino acid substitutions relative to 3E10-VH-CDR1a (SEQ ID NO:16),
(e) a VH CDR2 comprising an amino acid sequence having no more than two amino acid substitutions relative to 3E10-VH-CDR2 (SEQ ID NO:4), and
(f) a VH CDR3 comprising an amino acid sequence having no more than two amino acid substitutions relative to 3E10-VH-CDR3 (SEQ ID NO:5); and
the pharmaceutical composition comprises a molar ratio of (i) 3E10 antibody or antigen-binding fragment thereof to (ii) therapeutic polynucleotide of at least 2:1.

86.-130. (canceled)


SEQUENCE LISTING (110 sequences)

SEQ ID NO: 1 - protein, 446 aa, Artificial sequence
  Sequence is synthesized
EVQLVESGGGLVKPGGSRKLSCAASGFTFSDYGMHWVRQAPEKGLEWVAYISSGSSTIYYADTVKGRFTISRDNAKNTLFLQMTSLRSEDTAMYYCARRGLLLDYWGQGTTLTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 116 aa, Artificial sequence
  Sequence is synthesized
EVQLVESGGGLVKPGGSRKLSCAASGFTFSDYGMHWVRQAPEKGLEWVAYISSGSSTIYYADTVKGRFTISRDNAKNTLFLQMTSLRSEDTAMYYCARRGLLLDYWGQGTTLTVSS

SEQ ID NO: 3 - protein, 5 aa, Artificial sequence
  Sequence is synthesized
DYGMH

SEQ ID NO: 4 - protein, 17 aa, Artificial sequence
  Sequence is synthesized
YISSGSSTIYYADTVKG

SEQ ID NO: 5 - protein, 7 aa, Artificial sequence
  Sequence is synthesized
RGLLLDY

SEQ ID NO: 6 - protein, 19 aa, Artificial sequence
  Sequence is synthesized
MGWSCIILFLVATATGVHS

SEQ ID NO: 7 - protein, 218 aa, Artificial sequence
  Sequence is synthesized
DIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 111 aa, Artificial sequence
  Sequence is synthesized
DIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIK

SEQ ID NO: 9 - protein, 15 aa, Artificial sequence
  Sequence is synthesized
RASKSVSTSSYSYMH

SEQ ID NO: 10 - protein, 7 aa, Artificial sequence
  Sequence is synthesized
YASYLES

SEQ ID NO: 11 - protein, 9 aa, Artificial sequence
  Sequence is synthesized
QHSREFPWT

SEQ ID NO: 12 - protein, 19 aa, Artificial sequence
  Sequence is synthesized
MGWSCIILFLVATATGVHS

SEQ ID NO: 13 - protein, 446 aa, Artificial sequence
  Sequence is synthesized
EVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 116 aa, Artificial sequence
  Sequence is synthesized
EVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSS

SEQ ID NO: 15 - protein, 5 aa, Artificial sequence
  Sequence is synthesized
NYGMH

SEQ ID NO: 16 - protein, 5 aa, Artificial sequence
  Sequence is synthesized
  misc_feature (1)..(1): Xaa at position 1 is D or N
XYGMH

SEQ ID NO: 17 - protein, 17 aa, Artificial sequence
  Sequence is synthesized
YISSGSSTIYYADTVKG

SEQ ID NO: 18 - protein, 7 aa, Artificial sequence
  Sequence is synthesized
RGLLLDY

SEQ ID NO: 19 - protein, 19 aa, Artificial sequence
  Sequence is synthesized
MGWSCIILFLVATATGVHS

SEQ ID NO: 20 - protein, 218 aa, Artificial sequence; identical to SEQ ID NO: 7
  Sequence is synthesized

SEQ ID NO: 21 - protein, 111 aa, Artificial sequence; identical to SEQ ID NO: 8
  Sequence is synthesized

SEQ ID NO: 22 - protein, 15 aa, Artificial sequence
  Sequence is synthesized
RASKSVSTSSYSYMH

SEQ ID NO: 23 - protein, 7 aa, Artificial sequence
  Sequence is synthesized
YASYLES

SEQ ID NO: 24 - protein, 9 aa, Artificial sequence
  Sequence is synthesized
QHSREFPWT

SEQ ID NO: 25 - protein, 19 aa, Artificial sequence
  Sequence is synthesized
MGWSCIILFLVATATGVHS

SEQ ID NO: 26 - protein, 17 aa, Artificial sequence
  Sequence is synthesized
YISSGSSTIYYADSVKG

SEQ ID NO: 27 - protein, 17 aa, Artificial sequence
  Sequence is synthesized
YISSSSSTIYYADSVKG

SEQ ID NO: 28 - protein, 15 aa, Artificial sequence
  Sequence is synthesized
RASKSVSTSSYSYLA

SEQ ID NO: 29 - protein, 15 aa, Artificial sequence
  Sequence is synthesized
RASKTVSTSSYSYMH

SEQ ID NO: 30 - protein, 7 aa, Artificial sequence
  Sequence is synthesized
YASYLQS

SEQ ID NO: 31 - protein, 17 aa, Artificial sequence
  Sequence is synthesized
  misc_feature (5)..(5): Xaa at position 5 may be any naturally- occurring amino acid
  misc_feature (14)..(14): Xaa at position 14 may be any naturally- occurring amino acid
YISSXSSTIYYADXVKG

SEQ ID NO: 32 - protein, 15 aa, Artificial sequence
  Sequence is synthesized
  misc_feature (5)..(5): Xaa at position 5 may be any naturally- occurring amino acid
  misc_feature (14)..(14): Xaa at position 14 may be any naturally- occurring amino acid
  misc_feature (15)..(15): Xaa at position 15 may be any naturally- occurring amino acid
RASKXVSTSSYSYXX

SEQ ID NO: 33 - protein, 7 aa, Artificial sequence
  Sequence is synthesized
  misc_feature (6)..(6): Xaa at position 6 may be any naturally- occurring amino acid
YASYLXS

SEQ ID NO: 34 - protein, 5 aa, Artificial sequence
  Sequence is synthesized
QYGMH

SEQ ID NO: 35 - protein, 5 aa, Artificial sequence
  Sequence is synthesized
EYGMH

SEQ ID NO: 36 - protein, 5 aa, Artificial sequence
  Sequence is synthesized
  misc_feature (1)..(1): Xaa at position 1 is D or N
  misc_feature (5)..(5): Xaa at position 5 is K or R
XYGMX

SEQ ID NO: 37 - protein, 5 aa, Artificial sequence
  Sequence is synthesized
  misc_feature (5)..(5): Xaa at position 5 is K or R
QYGMX

SEQ ID NO: 38 - protein, 5 aa, Artificial sequence
  Sequence is synthesized
  misc_feature (5)..(5): Xaa at position 5 is K or R
EYGMX

SEQ ID NO: 39 - protein, 17 aa, Artificial sequence
  Sequence is synthesized
YISSGSSTIYYAETVKG

SEQ ID NO: 40 - protein, 17 aa, Artificial sequence
  Sequence is synthesized
  misc_feature (16)..(16): Xaa at position 16 is R or H
YISSGSSTIYYADTVXG

SEQ ID NO: 41 - protein, 17 aa, Artificial sequence
  Sequence is synthesized
  misc_feature (16)..(16): Xaa at position 16 is R or H
YISSGSSTIYYAETVXG

SEQ ID NO: 42 - protein, 7 aa, Artificial sequence
  Sequence is synthesized
  misc_feature (1)..(1): Xaa at position 1 is K or H
XGLLLDY

SEQ ID NO: 43 - protein, 7 aa, Artificial sequence
  Sequence is synthesized
RGLLLEY

SEQ ID NO: 44 - protein, 7 aa, Artificial sequence
  Sequence is synthesized
  misc_feature (1)..(1): Xaa at position 1 is K or H
XGLLLEY

SEQ ID NO: 45 - protein, 15 aa, Artificial sequence
  Sequence is synthesized
  misc_feature (1)..(1): Xaa at position 1 is K or H
XASKSVSTSSYSYMH

SEQ ID NO: 46 - protein, 15 aa, Artificial sequence
  Sequence is synthesized
  misc_feature (4)..(4): Xaa at position 4 is R or H
RASXSVSTSSYSYMH

SEQ ID NO: 47 - protein, 15 aa, Artificial sequence
  Sequence is synthesized
  misc_feature (15)..(15): Xaa at position 15 is K or R
RASKSVSTSSYSYMX

SEQ ID NO: 48 - protein, 15 aa, Artificial sequence
  Sequence is synthesized
  misc_feature (1)..(1): Xaa at position 1 is K or H
  misc_feature (4)..(4): Xaa at position 4 is R or H
XASXSVSTSSYSYMH

SEQ ID NO: 49 - protein, 15 aa, Artificial sequence
  Sequence is synthesized
  misc_feature (1)..(1): Xaa at position 1 is K or H
  misc_feature (15)..(15): Xaa at position 15 is K or R
XASKSVSTSSYSYMX

SEQ ID NO: 50 - protein, 15 aa, Artificial sequence
  Sequence is synthesized
  misc_feature (4)..(4): Xaa at position 4 is R or H
  misc_feature (15)..(15): Xaa at position 15 is K or R
RASXSVSTSSYSYMX

SEQ ID NO: 51 - protein, 7 aa, Artificial sequence
  Sequence is synthesized
YASYLDS

SEQ ID NO: 52 - protein, 9 aa, Artificial sequence
  Sequence is synthesized
  misc_feature (2)..(2): Xaa at position 2 is K or R
QXSREFPWT

SEQ ID NO: 53 - protein, 9 aa, Artificial sequence
  Sequence is synthesized
  misc_feature (4)..(4): Xaa at position 4 is K or H
QHSXEFPWT

SEQ ID NO: 54 - protein, 9 aa, Artificial sequence
  Sequence is synthesized
QHSRDFPWT

SEQ ID NO: 55 - protein, 9 aa, Artificial sequence
  Sequence is synthesized
  misc_feature (2)..(2): Xaa at position 2 is K or R
  misc_feature (4)..(4): Xaa at position 4 is K or H
QXSXEFPWT

SEQ ID NO: 56 - protein, 9 aa, Artificial sequence
  Sequence is synthesized
  misc_feature (2)..(2): Xaa at position 2 is K or R
QXSRDFPWT

SEQ ID NO: 57 - protein, 9 aa, Artificial sequence
  Sequence is synthesized
  misc_feature (4)..(4): Xaa at position 4 is K or H
QHSXDFPWT

SEQ ID NO: 58 - protein, 5 aa, Artificial sequence
  Sequence is synthesized
  misc_feature (1)..(1): Xaa at position 1 is any of D, E, N, Q, R, and K
  misc_feature (5)..(5): Xaa at position 5 is any one of K, R, and H
XYGMX

SEQ ID NO: 59 - protein, 17 aa, Artificial sequence
  Sequence is synthesized
  misc_feature (5)..(5): Xaa at position 5 is G or S
  misc_feature (13)..(13): Xaa at position 13 is D or E
  misc_feature (14)..(14): Xaa at position 14 is T or S
  misc_feature (16)..(16): Xaa at position 16 is any one of K, R, and H
YISSXSSTIYYAXXVXG

SEQ ID NO: 60 - protein, 7 aa, Artificial sequence
  Sequence is synthesized
  misc_feature (1)..(1): Xaa at position 1 is any one of K, R, and H
  misc_feature (6)..(6): Xaa at position 6 is D or E
XGLLLXY

SEQ ID NO: 61 - protein, 15 aa, Artificial sequence
  Sequence is synthesized
  misc_feature (1)..(1): Xaa at position 1 is any one of K, R, and H
  misc_feature (4)..(4): Xaa at position 4 is any one of K, R, and H
  misc_feature (5)..(5): Xaa at position 5 is T or S
  misc_feature (14)..(14): Xaa at position 14 is M or L
  misc_feature (15)..(15): Xaa at position 15 is any one of K, R, H, and A
XASXXVSTSSYSYXX

SEQ ID NO: 62 - protein, 7 aa, Artificial sequence
  Sequence is synthesized
  misc_feature (6)..(6): Xaa at position 6 is any one or D, E, N, and Q
YASYLXS

SEQ ID NO: 63 - protein, 9 aa, Artificial sequence
  Sequence is synthesized
  misc_feature (2)..(2): Xaa at position 1 is any one or K, R, and H
  misc_feature (4)..(4): Xaa at position 4 is any one of K, R, and H
  misc_feature (5)..(5): Xaa at position 5 is D or E
QXSXXFPWT

SEQ ID NO: 64 - protein, 116 aa, Artificial sequence
  Sequence is synthesized
EVQLVESGGGLVQPGGSLRLSCSASGFTFSNYGMHWVRQAPGKGLEYVSYISSGSSTIYYADTVKGRFTISRDNSKNTLYLQMSSLRAEDTAVYYCVKRGLLLDYWGQGTLVTVSS

SEQ ID NO: 65 - protein, 116 aa, Artificial sequence
  Sequence is synthesized
EVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSS

SEQ ID NO: 66 - protein, 116 aa, Artificial sequence
  Sequence is synthesized
EVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSS

SEQ ID NO: 67 - protein, 116 aa, Artificial sequence
  Sequence is synthesized
EVQLVQSGGGLIQPGGSLRLSCAASGFTFSNYGMHWVRQAPGKGLEWVSYISSGSSTIYYADSVKGRFTISRDNSKNTLYLQMNSLRAEDTAVYYCARRGLLLDYWGQGTTVTVSS

SEQ ID NO: 68 - protein, 116 aa, Artificial sequence
  Sequence is synthesized
EVQLVESGGGLIQPGGSLRLSCAASGFTFSNYGMHWVRQAPGKGLEWVSYISSGSSTIYYADSVKGRFTISRDNSKNTLYLQMTSLRAEDTAVYYCARRGLLLDYWGQGTTLTVSS

SEQ ID NO: 69 - protein, 116 aa, Artificial sequence
  Sequence is synthesized
EVQLQESGGGVVQPGGSLRLSCAASGFTFSNYGMHWIRQAPGKGLEWVSYISSGSSTIYYADSVKGRFTISRDNSKNTLYLQMNSLRSEDTAVYYCARRGLLLDYWGQGTLVTVSS

SEQ ID NO: 70 - protein, 116 aa, Artificial sequence
  Sequence is synthesized
EVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 71 - protein, 116 aa, Artificial sequence
  Sequence is synthesized
EVQLVESGGGVVQPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 72 - protein, 116 aa, Artificial sequence
  Sequence is synthesized
EVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 73 - protein, 116 aa, Artificial sequence
  Sequence is synthesized
EVQLVESGGGDVKPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 74 - protein, 111 aa, Artificial sequence
  Sequence is synthesized
DIVLTQSPASLAVSPGQRATITCRASKSVSTSSYSYMHWYQQKPGQPPKLLIYYASYLESGVPARFSGSGSGTDFTLTINPVEANDTANYYCQHSREFPWTFGQGTKVEIK

SEQ ID NO: 75 - protein, 111 aa, Artificial sequence
  Sequence is synthesized
DIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIK

SEQ ID NO: 76 - protein, 111 aa, Artificial sequence
  Sequence is synthesized
DIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIK

SEQ ID NO: 77 - protein, 111 aa, Artificial sequence
  Sequence is synthesized
DIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIK

SEQ ID NO: 78 - protein, 111 aa, Artificial sequence
  Sequence is synthesized
DIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIK

SEQ ID NO: 79 - protein, 111 aa, Artificial sequence
  Sequence is synthesized
DIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIK

SEQ ID NO: 80 - protein, 111 aa, Artificial sequence
  Sequence is synthesized
DIQMTQSPSSLSASLGDRATITCRASKTVSTSSYSYMHWYQQKPGQAPKLLIKYASYLESGVPSRFSGSGSGTDFTLTISSLQPEDFATYYCQHSREFPWTFGQGTKVEIK

SEQ ID NO: 81 - protein, 111 aa, Artificial sequence
  Sequence is synthesized
DIQMTQSPSSLSASVGDRVTITCRASKSVSTSSYSYLAWYQQKPEKAPKLLIKYASYLQSGVPSRFSGSGSGTDFTLTISSLQPEDFATYYCQHSREFPWTFGAGTKLELK

SEQ ID NO: 82 - protein, 111 aa, Artificial sequence
  Sequence is synthesized
DIQMTQSPSSLSASVGDRVTISCRASKSVSTSSYSYMHWYQQKPEKAPKLLIKYASYLQSGVPSRFSGSGSGTDFTLTISSLQPEDVATYYCQHSREFPWTFGAGTKLELK

SEQ ID NO: 83 - protein, 515 aa, Artificial sequence
  Sequence is synthesized
DIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSS

SEQ ID NO: 84 - protein, 515 aa, Artificial sequence
  Sequence is synthesized
DIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSSASTKGPSVFPLAPLESSGSDIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSS

SEQ ID NO: 85 - protein, 515 aa, Artificial sequence
  Sequence is synthesized
DIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSS

SEQ ID NO: 86 - protein, 515 aa, Artificial sequence
  Sequence is synthesized
DIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 87 - protein, 515 aa, Artificial sequence
  Sequence is synthesized
DIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 88 - protein, 515 aa, Artificial sequence
  Sequence is synthesized
DIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 89 - protein, 515 aa, Artificial sequence
  Sequence is synthesized
DIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 90 - protein, 515 aa, Artificial sequence
  Sequence is synthesized
DIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 91 - protein, 515 aa, Artificial sequence
  Sequence is synthesized
DIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 92 - protein, 515 aa, Artificial sequence
  Sequence is synthesized
DIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSSASTKGPSVFPLAPLESSGSDIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSS

SEQ ID NO: 93 - protein, 515 aa, Artificial sequence
  Sequence is synthesized
DIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSSASTKGPSVFPLAPLESSGSDIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSS

SEQ ID NO: 94 - protein, 515 aa, Artificial sequence
  Sequence is synthesized
DIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGDVKPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGDVKPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 95 - protein, 515 aa, Artificial sequence
  Sequence is synthesized
DIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSSASTKGPSVFPLAPLESSGSDIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 96 - protein, 515 aa, Artificial sequence
  Sequence is synthesized
DIQMTQSPSSLSASLGDRATITCRASKTVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSSASTKGPSVFPLAPLESSGSDIQMTQSPSSLSASLGDRATITCRASKTVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSS

SEQ ID NO: 97 - protein, 515 aa, Artificial sequence
  Sequence is synthesized
DIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGVVQPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGVVQPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 98 - protein, 515 aa, Artificial sequence
  Sequence is synthesized
DIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSSASTKGPSVFPLAPLESSGSDIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 99 - protein, 8 aa, Artificial sequence
  Sequence is synthesized
GFTFSDYG

SEQ ID NO: 100 - protein, 8 aa, Artificial sequence
  Sequence is synthesized
GFTFSNYG

SEQ ID NO: 101 - protein, 8 aa, Artificial sequence
  Sequence is synthesized
ISSGSSTI

SEQ ID NO: 102 - protein, 8 aa, Artificial sequence
  Sequence is synthesized
ISSSSSTI

SEQ ID NO: 103 - protein, 9 aa, Artificial sequence
  Sequence is synthesized
ARRGLLLDY

SEQ ID NO: 104 - protein, 10 aa, Artificial sequence
  Sequence is synthesized
KSVSTSSYSY

SEQ ID NO: 105 - protein, 10 aa, Artificial sequence
  Sequence is synthesized
KTVSTSSYSY

SEQ ID NO: 106 - protein, 3 aa, Artificial sequence
  Sequence is synthesized
YAS

SEQ ID NO: 107 - protein, 9 aa, Artificial sequence
  Sequence is synthesized
QHSREFPWT

SEQ ID NO: 108 - protein, 115 aa, Artificial sequence
  Sequence is synthesized
EVQLVESGGGLVKPGGSRKLSCAASGFTFSDYGMHWVRQAPEKGLEWVAYISSGSSTIYYADTVKGRFTISRDNAKNTLFLQMTSLRSEDTAMYYCARRGLLLDYWGQGTTLTVS

SEQ ID NO: 109 - protein, 132 aa, Artificial sequence
  Sequence is synthesized
DIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIKRADAAPGGGGSGGGGSGGGGS

SEQ ID NO: 110 - RNA, 720 nt, Artificial sequence
  Sequence is synthesized
auggugagcaagggcgaggagcuguucaccgggguggugcccauccuggucgagcuggacggcgacguaaacggccacaaguucagcguguccggcgagggcgagggcgaugccaccuacggcaagcugacccugaaguucaucugcaccaccggcaagcugcccgugcccuggcccacccucgugaccacccugaccuacggcgugcagugcuucagccgcuaccccgaccacaugaagcagcacgacuucuucaaguccgccaugcccgaaggcuacguccaggagcgcaccaucuucuucaaggacgacggcaacuacaagacccgcgccgaggugaaguucgagggcgacacccuggugaaccgcaucgagcugaagggcaucgacuucaaggaggacggcaacauccuggggcacaagcuggaguacaacuacaacagccacaacgucuauaucauggccgacaagcagaagaacggcaucaaggugaacuucaagauccgccacaacaucgaggacggcagcgugcagcucgccgaccacuaccagcagaacacccccaucggcgacggccccgugcugcugcccgacaaccacuaccugagcacccaguccgcccugagcaaagaccccaacgagaagcgcgaucacaugguccugcuggaguucgugaccgccgccgggaucacucucggcauggacgagcuguacaaguaa